(19) United States
(12) Patent Application Publication
(10) Pub. No.: US 2024/0307448 A1
(43) Pub. Date: Sep. 19, 2024

(54) COMPOSITIONS AND METHODS FOR TREATING CANCER WITH SELF-DRIVING CHIMERIC ANTIGEN RECEPTORS

(71) Applicant: Lentigen Technology, Inc., Gaithersburg, MD (US)

(72) Inventors: Dina Schneider, Potomac, MD (US); Boro Dropulic, Ellicott City, MD (US); Brian Robert Webster, Köln (DE)

(21) Appl. No.: 18/624,207

(22) Filed: Apr. 2, 2024

Related U.S. Application Data
(62) Division of application No. 17/436,432, filed on Sep. 3, 2021, now Pat. No. 11,969,443.
(60) Provisional application No. 62/954,161, filed on Dec. 27, 2019.
(60) Provisional application No. 62/814,759, filed on Mar. 6, 2019.

(51) Int. Cl. A61K 35/17; C07K 14/705; C07K 14/725; C07K 16/28

(52) U.S. Cl. CPC A61K 35/17; C07K 14/7051; C07K 14/70517; C07K 14/70578; C07K 16/2803; C07K 2317/622; C07K 2319/03

(57) ABSTRACT

Self-driving surface antigen-regulated promoter-therapeutic payload constructs containing antigen binding domains are disclosed. Nucleic acids, recombinant expression vectors, host cells, antigen binding fragments, and pharmaceutical compositions, relating to the surface antigen-regulated promoter-therapeutic payload constructs are also disclosed. Methods of treating or preventing cancer in a subject, and methods of making self-driving surface antigen-regulated promoter-therapeutic payload constructs in T-cells are also disclosed.

### CROSS-REFERENCE TO RELATED APPLICATIONS

This PCT patent application claims priority to U.S. Provisional Patent Application No. 62/954,161 filed on Dec. 27, 2019, and U.S. Provisional Patent Application No. 62/814,759 filed on Mar. 6, 2019, the entire contents of each of which are incorporated herein by reference.

### FIELD OF THE DISCLOSURE

This application relates to the field of cancer, particularly to inducible promoters connected to therapeutic payloads and methods of use thereof.

### BACKGROUND

Cancer is one of the most deadly threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making treatment extremely difficult.

Multiple myeloma (“MM”) is a debilitating and often incurable disease, with over 30,000 new cases diagnosed in the U.S. every year (source: MM research foundation). MM is the second most prevalent blood cancer in the U.S., after Non-Hodgkin's Lymphoma (“NHL”), (Smith L, McCourt O, Henrich M, et al. Multiple myeloma and physical activity: a scoping review. BMJ Open. 2015; 5: e009576). MM impacts plasma cells in the bone marrow, and may lead to bone marrow failure and patient death (National Cancer Institute. A snapshot of myeloma. Nov. 5, 2014. see the world wide web at cancer.gov). Complications of myeloma include bone pain, bone loss, anemia, immunosuppression, kidney disfunction, neuropathy (Mayo Clinic staff. Diseases and conditions: multiple myeloma: treatments and drugs. Dec. 4, 2015).

First line therapy for MM includes proteasome inhibitors, immunomodulatory drugs, steroids, histone deacetylase (“HDAC”) inhibitors, and chemotherapy. These approaches are aimed at killing MM cells, however, many of them are also associated with broad immunosuppression and systemic toxicities.

Chimeric antigen receptor T-cell (CAR T) technology has brought great advancement to the treatment of hematologic malignancies like MM, and holds great promise for the treatment of solid tumors. However, two presently unresolved drawbacks of this technology are insufficient CAR T potency and suboptimal responses on the one hand, and detrimental side effects stemming from excessive CAR T activation, such as cytokine release syndrome on the other hand. The purpose of this invention is to address both issues by creating CAR T moieties and other therapeutic payloads that can be naturally fine-tuned based on tumor burden and inflammatory milieu in the patient, at any given time in the course of treatment, and in any local microenvironment.

Chimeric Antigen Receptors (CARs) are hybrid molecules comprising three essential units: (1) an extracellular antigen-binding motif, (2) linking/transmembrane motifs, and (3) intracellular T-cell signaling motifs (Long A H, Haso W M, Orentas R J. Lessons learned from a highly-active CD22-specific chimeric antigen receptor. Oncoimmunology. 2013; 2 (4):e23621). The antigen-binding motif of a CAR is commonly fashioned after an single chain Fragment variable (ScFv), the minimal binding domain of an immunoglobulin (Ig) molecule. Alternate antigen-binding motifs, such as receptor ligands (e.g., IL-13 has been engineered to bind tumor expressed IL-13 receptor), intact immune receptors, library-derived peptides, and innate immune system effector molecules (such as NKG2D) also have been engineered. Alternate cell targets for CAR expression (such as NK or gamma-delta T cells) are also under development (Brown C E et al. Clin Cancer Res. 2012; 18(8):2199-209; Lehner M et al. PLoS One. 2012; 7 (2):e31210). There remains significant work with regard to defining the most active T-cell population to transduce with CAR vectors, determining the optimal culture and expansion techniques, and defining the molecular details of the CAR protein structure itself.

The linking motifs of a CAR can be a relatively stable structural domain, such as the constant domain of IgG, or designed to be an extended flexible linker. Structural motifs, such as those derived from IgG constant domains, can be used to extend the ScFv binding domain away from the T-cell plasma membrane surface. This may be important for some tumor targets where the binding domain is particularly close to the tumor cell surface membrane (such as for the disialoganglioside GD2; Orentas et al., unpublished observations). To date, the signaling motifs used in CARs always include the CD3-ζ chain because this core motif is the key signal for T cell activation. The first reported second-generation CARs featured CD28 signaling domains and the CD28 transmembrane sequence. This motif was used in third-generation CARs containing CD137 (4-1BB) signaling motifs as well (Zhao Y et al. J Immunol. 2009; 183 (9): 5563-74). With the advent of new technology, the activation of T cells with beads linked to anti-CD3 and anti-CD28 antibody, and the presence of the canonical “signal 2” from CD28 was no longer required to be encoded by the CAR itself. Using bead activation, third-generation vectors were found to be not superior to second-generation vectors in in vitro assays, and they provided no clear benefit over second-generation vectors in mouse models of leukemia (Haso W, Lee D W, Shah N N, Stetler-Stevenson M, Yuan C M, Pastan I H, Dimitrov D S, Morgan R A, FitzGerald D J, Barrett D M, Wayne A S, Mackall C L, Orentas R J. Anti-CD22-chimeric antigen receptors targeting B cell precursor acute lymphoblastic leukemia, Blood. 2013; 121 (7):1165-74; Kochenderfer J N et al. Blood. 2012; 119 (12):2709-20). This is borne out by the clinical success of CD19-specific CARs that are in a second generation CD28/CD3-ζ (Lee D W et al. American Society of Hematology Annual Meeting. New Orleans, LA; Dec. 7-10, 2013) and a CD137/CD3-ζ signaling format (Porter D L et al. N Engl J Med. 2011; 365 (8): 725-33). In addition to CD137, other tumor necrosis factor receptor superfamily members such as OX40 also are able to provide important persistence signals in CAR-transduced T cells (Yvon E et al. Clin Cancer Res. 2009; 15(18):5852-60). Equally important are the culture conditions under which the CAR T-cell populations were cultured.

T-cell-based immunotherapy has become a new frontier in synthetic biology; multiple promoters and gene products are envisioned to steer these highly potent cells to the tumor microenvironment, where T cells can both evade negative regulatory signals and mediate effective tumor killing. The elimination of unwanted T cells through the drug-induced dimerization of inducible caspase 9 constructs with AP1903 demonstrates one way in which a powerful switch that can control T-cell populations can be initiated pharmacologically (Di Stasi A et al. N Engl J Med. 2011; 365(18):1673-83). The creation of effector T-cell populations that are immune to the negative regulatory effects of transforming growth factor-β by the expression of a dominant negative receptor further demonstrates that degree to which effector T cells can be engineered for optimal antitumor activity (Foster A E et al. J Immunother. 2008; 31(5):500-5). Thus, while it appears that CARs can trigger T-cell activation in a manner similar to an endogenous T-cell receptor, a major impediment to the clinical application of this technology to date has been limited in vivo expansion of CAR+ T cells, rapid disappearance of the cells after infusion, and disappointing clinical activity.

To date, prior art CAR therapeutic approaches employ CAR T constructs under the control of a constitutive promoter such as human elongation factor 1 alpha (EF1α), phosphoglycerate kinase (PGK), murine leukemia virus (MuLV), murine stem cell virus (MSCV), or other constitutive promoters known in the art, which constitutive promoters are often expressed at a high level, or induced artificially (via small molecule or a soluble component). These approaches are maladjusted to the dynamics of antigen expression, which may lead to, on a cellular level, an excessive or insufficient effector cell response, and on an organism level, to undertreatment or over-treatment and toxicity for the patient.

Accordingly, there is an urgent and long felt need in the art for discovering novel compositions and methods for treatment of MM and CLL using an approach that can exhibit specific and efficacious anti-tumor effect without the aforementioned shortcomings (e.g. high toxicity, insufficient efficacy).

The present invention addresses these needs by providing compositions and therapeutic methods that can be used to treat cancers and other diseases and/or conditions. In particular, the present invention as disclosed and described herein provides inducible promoter-therapeutic payload constructs that may be used for the treatment of diseases, disorders or conditions associated with dysregulated expression of various antigens, for example and not by way of limitation, mesothelin, CD33, CD19, CD19/CD20, CD22, CD19/CD22, ROR1, CD123, or CD38, or a combination thereof, and which inducible promoter-therapeutic payload constructs contain antigen-specific binding domains that exhibit a high surface expression on transduced T cells, and exhibit a high degree of cytolysis and transduced T cell in vivo expansion and persistence. Furthermore, the present invention as disclosed and described herein provides for the self-driving regulation of such therapeutic payloads by utilizing a surface antigen-regulated inducible promoter which adjusts the level of expression of the one or more therapeutic payloads, dependent upon the level of expression of surface antigen on the target cell.

### SUMMARY

The present invention as disclosed and described herein is based upon the unexpected discovery that modulation or adjustment of a surface antigen-regulated promoter's expression of a therapeutic payload construct can be directly correlated with the activity of the therapeutic payload, and therefore the level of expression of surface antigen in a target cell's milieu.

Novel self-driving inducible promoter-payload therapeutic constructs are provided herein comprising a therapeutic payload operably connected to a surface antigen-regulated inducible promoter which adjusts the level of expression of the one or more therapeutic payloads, dependent upon the level of expression of surface antigen on the target cell.

Without being limited to any particular mechanism of action, as used herein, a “self-driving” surface antigen-regulated inducible promoter refers to the utilization of a surface antigen-regulated inducible promoter to drive the therapeutic payloads to provide for a low basal level of the therapeutic payload surface expression in the absence of tumor target antigen expression. In the presence of activating target antigen on the surface of a target cell, the therapeutic payload is activated which triggers activation of the applicable signal transduction pathway(s), thereby activating the signal mediator(s) of the surface antigen-regulated inducible promoter thereby leading to increased expression of the therapeutic payload above the basal level of expression.

In this manner, a positive feed-back loop is created whereby higher expression of a given target antigen leads to higher expression of the therapeutic payload and vice versa thereby leading to efficient regulation of therapeutic payload expression to achieve a T-cell response precisely tailored to the level of target present at the specific site and time. Heightened therapeutic payload expression leads to an optimal anti-tumor activity and rapid elimination of target tumor cells. As tumor cells are reduced/removed in amount, the level of the therapeutic payload expression returns to its basal level of expression.

In one aspect, an isolated nucleic acid molecule is provided herein encoding a therapeutic payload operably connected to a surface antigen-regulated inducible promoter, and wherein said surface antigen-regulated inducible promoter adjusts the level of expression of the one or more therapeutic payloads, dependent upon the level of expression of surface antigen on the target cell thereby achieving a T-cell response precisely regulated to the level of target antigen present in the tumor milieu.

In one embodiment, an isolated nucleic acid molecule is provided herein encoding a therapeutic payload operably connected to a surface antigen-regulated inducible promoter comprising a nucleotide sequence comprising SEQ ID NO: 137 and 138, or a combination thereof, and wherein said surface antigen-regulated inducible promoter adjusts the level of expression of the one or more therapeutic payloads, dependent upon the level of expression of surface antigen on the target cell thereby achieving a T-cell response precisely regulated to the level of target antigen present in the tumor milieu.

In one aspect, self-driving surface antigen-regulated inducible promoter-therapeutic payload constructs comprising at least one therapeutic payload comprising chimeric antigen receptors (CARs), cytokines, chemokines, trafficking receptors, bispecific antibodies, neutralizing/blocking antibodies, T-cell stimulatory receptors, truncated inhibitory receptors, hybrid inhibitory/activating receptors, anti-apoptotic proteins, shRNA, or protease, or a combination thereof, operably connected to a surface antigen-regulated inducible promoter comprising a STAT5 response element, AP-1 response element, or NF kappa B response element, or a combination thereof, which surface antigen-regulated inducible promoter adjusts the level of expression of the one or more therapeutic payloads, dependent upon the level of expression of surface antigen on the target cell are provided herein, as well as host cells (e.g., T-cells) expressing the surface antigen-regulated inducible promoter-therapeutic payload constructs, and nucleic acid molecules encoding the surface antigen-regulated inducible promoter therapeutic payload constructs.

In one embodiment, the one or more therapeutic payloads (for example, and not by way of limitation, chimeric antigen receptors (CARs), cytokines, chemokines, trafficking receptors, bispecific antibodies, neutralizing/blocking antibodies, T-cell stimulatory receptors, truncated inhibitory receptors, hybrid inhibitory/activating receptors, anti-apoptotic proteins, shRNA, or protease-based) are expressed under the control of the surface antigen-regulated inducible promoter, with separation of the one or more therapeutic payloads by a 2A ribosomal skip sequence or an internal ribosome entry sequence (IRES) or a combination thereof.

In one aspect, the surface antigen-regulated inducible promoter-therapeutic payload constructs disclosed herein may comprise, for example, and not by way of limitation, a CAR, which may comprise either a single molecule expressed on the effector cell surface, or may comprise an effector cell-expressed signaling module and a soluble targeting module, such that when the soluble targeting module binds to the cell-expressed signaling module, a complete functional CAR is formed. The CARs exhibit a high surface expression on transduced T cells, with a high degree of cytolysis and transduced T cell expansion and persistence in vivo. Methods of using the disclosed CARs, host cells, and nucleic acid molecules are also provided, for example, to treat a cancer in a subject. Methods of using the disclosed surface antigen-regulated inducible promoter-therapeutic payload constructs, host cells, and nucleic acid molecules are also provided, for example, to regulate expression of the therapeutic payload.

In one aspect, an isolated polynucleotide encoding a CAR-based surface antigen-regulated inducible promoter-therapeutic payload is provided, wherein the therapeutic payload is operably connected to a surface antigen-regulated inducible promoter, and wherein the therapeutic payload comprises a CAR comprising a fragment selected from the group consisting of an Fab fragment, an F(ab′)₂ fragment, an Fv fragment, and a single chain Fv (ScFv).

In one embodiment, an isolated nucleic acid molecule encoding the CAR-based surface antigen-regulated inducible promoter-therapeutic payload is provided wherein the encoded extracellular antigen binding domain comprises at least one single chain variable fragment of an antibody that binds to mesothelin, CD33, CD19, CD19/CD20, CD22, ROR1, CD123, or CD38 antigen binding domain, or a combination thereof.

In another embodiment, an isolated nucleic acid molecule encoding the CAR-based surface antigen-regulated inducible promoter-therapeutic payload is provided wherein the encoded extracellular antigen binding domain comprises at least one heavy chain variable region of an antibody that binds to mesothelin, CD33, CD19, CD19/CD20, CD22, ROR1, CD123, or CD38 antigen binding domain, or a combination thereof.

In one aspect, an isolated polynucleotide encoding a surface antigen-regulated inducible promoter-therapeutic payload construct is provided, wherein the therapeutic payload is operably connected to a surface antigen-regulated inducible promoter, and wherein the therapeutic payload comprises a CAR comprising, from N-terminus to C-terminus, at least one extracellular binding domain comprising a mesothelin, CD33, CD19, CD19/CD20, CD22, CD19/22, ROR1, CD123, or CD38 antigen binding domain, or a combination thereof; at least one transmembrane domain, and at least one intracellular signaling domain.

Thus, in one embodiment, an isolated polynucleotide encoding a CAR-based therapeutic payload operably connected to a surface antigen-regulated inducible promoter is provided wherein the at least one mesothelin, CD33, CD19, CD19/CD20, CD22, CD19/CD22, ROR1, CD123, or CD38 antigen binding domain of the CAR-based therapeutic payload comprises a nucleic acid sequence selected from the group consisting of SEQ ID NOs: 7, 9, 11, 15, 17, 19, 21, 23, 25, 87, 89, 91, 93, 95, 97, 115, 117, 119, 121, 123, 125, 127, 129, 131, 133, and 135.

In one embodiment, an isolated polynucleotide encoding a CAR-based therapeutic payload operably connected to at least one surface antigen-regulated inducible promoter is provided, wherein the at least one mesothelin, CD33, CD19, CD19/CD20, CD22, CD19/CD22, ROR1, CD123, or CD38 antigen binding domain of the CAR-based therapeutic payload comprises an amino acid sequence selected from the group consisting of SEQ ID NOs: 8, 10, 12, 16, 18, 20, 22, 24, 26, 88, 90, 92, 94, 98, 116, 118, 120, 122, 124, 126, 128, 130, 132, 134, and 136.

In one embodiment, an isolated polynucleotide encoding a CAR-based surface antigen-regulated inducible promoter-therapeutic payload construct is provided, wherein the therapeutic payload is operably connected to a surface antigen-regulated inducible promoter, and wherein the therapeutic payload comprises a CAR comprising, from N-terminus to C-terminus, at least one extracellular binding domain comprising a nucleic acid sequence selected from the group consisting of SEQ ID NOs: 7, 9, 11, 15, 17, 19, 21, 23, 25, 87, 89, 91, 93, 95, 97, 115, 117, 119, 121, 123, 125, 127, 129, 131, 133, and 135, or a combination thereof; at least one transmembrane domain, and at least one intracellular signaling domain.

In one embodiment, an isolated polynucleotide encoding a CAR-based surface antigen-regulated inducible promoter-therapeutic payload construct is provided, wherein the therapeutic payload is operably connected to a surface antigen-regulated inducible promoter, and wherein the therapeutic payload comprises a CAR comprising, from N-terminus to C-terminus, at least one extracellular binding domain comprising an amino acid sequence selected from the group consisting of SEQ ID NOs: 8, 10, 12, 16, 18, 20, 22, 24, 26, 88, 90, 92, 94, 98, 116, 118, 120, 122, 124, 126, 128, 130, 132, 134, and 136, or a combination thereof; at least one transmembrane domain, and at least one intracellular signaling domain.

In one embodiment, the targeting domain of the CAR-based surface antigen-regulated inducible promoter CAR-based therapeutic payload construct is expressed separately in the form of monoclonal antibody, ScFv Fab, Fab′2 and contains at least one mesothelin, CD33, CD19, CD19/CD20, CD22, CD19/CD22, ROR1, CD123, or CD38 antigen-targeting domain comprising a nucleic acid sequence selected from the group consisting of SEQ ID NOs: 7, 9, 11, 15, 17, 19, 21, 23, 25, 87, 89, 91, 93, 95, 97, 115, 117, 119, 121, 123, 125, 127, 129, 131, 133, and 135 coupled to an additional binding tag or epitope, wherein the effector-cell expressed component of the CAR contains a binding domain specifically directed to bind the tag or epitope expressed on the soluble CAR module, such as specific binding on the soluble component of the CAR to the cell bound component of the CAR forms the full functional CAR structure.

In another embodiment, the targeting domain of the CAR-based surface antigen-regulated inducible promoter-therapeutic payload construct is expressed separately in the form of a monoclonal antibody, ScFv Fab, Fab′2 and contains at least one mesothelin, CD33, CD19, CD19/CD20, CD22, CD19/CD22, ROR1, CD123, or CD38 antigen-targeting domain comprising a nucleic acid sequence selected from the group consisting of SEQ ID NOs: 7, 9, 11, 15, 17, 19, 21, 23, 25, 87, 89, 91, 93, 95, 97, 115, 117, 119, 121, 123, 125, 127, 129, 131, 133, and 135 and an additional ScFv, whereas the effector-cell expressed component of the CAR contains a tag or epitope specifically reactive with the additional ScFv expressed on the soluble CAR module, such as specific binding on the soluble component of the CAR to the cell bound component of the CAR forms the full functional CAR structure.

In yet another embodiment, an isolated nucleic acid molecule encoding the CAR-based surface antigen-regulated inducible promoter-therapeutic payload construct is provided wherein the encoded CAR extracellular antigen binding domain further comprises at least one lipocalin-based antigen binding antigen (anticalins) that binds to mesothelin, CD33, CD19, CD19/CD20, CD22, CD19/22, ROR1, CD123, or CD38 antigen binding domain, or a combination thereof.

In one embodiment, an isolated nucleic acid molecule encoding a CAR-based surface antigen-regulated inducible promoter-therapeutic payload construct is provided wherein the encoded extracellular mesothelin, CD33, CD19, CD19/CD20, CD22, CD19/22, ROR1, CD123, or CD38, antigen binding domain is connected to the transmembrane domain by a linker domain.

In another embodiment, an isolated nucleic acid molecule encoding a CAR-based surface antigen-regulated inducible promoter-therapeutic payload construct is provided wherein the encoded extracellular mesothelin, CD33, CD19, CD19/CD20, CD22, CD19/22, ROR1, CD123, or CD38 antigen binding domain is preceded by a sequence encoding a leader or signal peptide.

In yet another embodiment, an isolated nucleic acid molecule encoding a CAR-based surface antigen-regulated inducible promoter-therapeutic payload construct is provided comprising at least one antigen binding domain encoded by a nucleotide sequence comprising a nucleic acid sequence selected from the group consisting of SEQ ID NOs: 7, 9, 11, 15, 17, 19, 21, 23, 25, 87, 89, 91, 93, 95, 97, 115, 117, 119, 121, 123, 125, 127, 129, 131, 133, and 135, and wherein the CAR additionally encodes an extracellular antigen binding domain which targets an antigen that includes, but is not limited to, CD19, CD20, CD22, CD33, CD123, CD5, CD7, CD138, BCMA (CD269), ROR1, TSLPR, TEM-1, TEM-7, TEM-8, TEM-9, CD371, CD276, CD99, GPC2, GPC3, FGFR4, c-Met, PSMA, Glycolipid F77, EGFRvIII, GD-2, NY-ESO-1 TCR, MAGE A3 TCR, PRAME TCR, KRAS TCR or any combination thereof.

In certain embodiments, an isolated nucleic acid molecule encoding a CAR-based surface antigen-regulated inducible promoter-therapeutic payload construct is provided wherein the additionally encoded extracellular antigen binding domain comprises an anti-CD19 ScFv antigen binding domain, an anti-CD20 ScFv antigen binding domain, an anti-CD22 ScFv antigen binding domain, an anti-ROR1 ScFv antigen binding domain, an anti-mesothelin ScFv antigen binding domain, an anti-CD33 ScFv antigen binding domain, an anti-CD38 ScFv antigen binding domain, an anti-CD123 (IL3RA) ScFv antigen binding domain, an anti-CD138 ScFv antigen binding domain, an anti-BCMA (CD269) ScFv antigen binding domain, an anti-GPC2 ScFv antigen binding domain, an anti-GPC3 ScFv antigen binding domain, an anti-FGFR4 ScFv antigen binding domain, an anti-c-Met ScFv antigen binding domain, an anti-PMSA ScFv antigen binding domain, an anti-glycolipid F77 ScFv antigen binding domain, an anti-EGFRvIII ScFv antigen binding domain, an anti-GD-2 ScFv antigen binding domain, an anti-NY-ESo-1 TCR ScFv antigen binding domain, an anti-MAGE A3 TCR ScFv antigen binding domain, or an amino acid sequence with 85%, 90%, 95%, 96%, 97%, 98% or 99% identity thereof, or any combination thereof.

In one aspect, the CAR-based surface antigen-regulated inducible promoter-therapeutic payload constructs provided herein further comprise a linker or spacer domain.

In one embodiment, an isolated nucleic acid molecule encoding a CAR-based surface antigen-regulated inducible promoter-therapeutic payload construct is provided wherein the extracellular antigen binding domain comprising mesothelin, CD33, CD19, CD19/CD20, CD22, CD19/22, ROR1, CD123, or CD38, or a combination thereof; the intracellular signaling domain; or both are connected to the transmembrane domain by a linker or spacer domain.

In one embodiment, an isolated nucleic acid molecule encoding a CAR-based surface antigen-regulated inducible promoter-therapeutic payload construct is provided wherein the encoded linker domain is derived from the extracellular domain of CD8, TNFRSF19, or CD28, and is linked to a transmembrane domain.

In another embodiment, an isolated nucleic acid molecule encoding a CAR-based surface antigen-regulated inducible promoter-therapeutic payload construct is provided wherein the encoded CAR further comprises a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, TNFRSF19 and CD154, or a combination thereof.

In yet another embodiment, an isolated nucleic acid molecule encoding a CAR-based surface antigen-regulated inducible promoter-therapeutic payload construct is provided wherein the encoded intracellular signaling domain further comprises a CD3 zeta intracellular domain.

In one embodiment, an isolated nucleic acid molecule encoding a CAR-based surface antigen-regulated inducible promoter-therapeutic payload construct is provided wherein the encoded intracellular signaling domain is arranged on a C-terminal side relative to the CD3 zeta intracellular domain.

In another embodiment, an isolated nucleic acid molecule encoding a CAR-based surface antigen-regulated inducible promoter-therapeutic payload construct is provided wherein the encoded at least one intracellular signaling domain comprises a costimulatory domain, a primary signaling domain, or a combination thereof.

In further embodiments, an isolated nucleic acid molecule encoding a CAR-based surface antigen-regulated inducible promoter-therapeutic payload construct is provided wherein the encoded at least one costimulatory domain comprises a functional signaling domain of OX40, CD70, CD27, CD28, CD5, ICAM-1, LFA-1 (CD11a/CD18), ICOS (CD278), DAP10, DAP12, and 4-1BB (CD137), or a combination thereof.

In one embodiment, an isolated nucleic acid molecule encoding a CAR-based surface antigen-regulated inducible promoter-therapeutic payload construct is provided that further contains a leader sequence or signal peptide wherein the leader or signal peptide nucleotide sequence comprises the nucleotide sequence of SEQ ID NO: 13, SEQ ID NO: 39, SEQ ID NO: 41, or SEQ ID NO: 43.

In yet another embodiment, an isolated nucleic acid molecule encoding a CAR-based surface antigen-regulated inducible promoter-therapeutic payload construct is provided wherein the encoded leader sequence comprises the amino acid sequence of SEQ ID NO: 14 SEQ ID NO: 40, SEQ ID NO: 42, or SEQ ID NO: 44.

In one aspect, a CAR-based surface antigen-regulated inducible promoter-therapeutic payload construct is provided herein comprising, from N-terminus to C-terminus, at least one antigen binding domain, at least one transmembrane domain, and at least one intracellular signaling domain.

In one embodiment, a CAR-based surface antigen-regulated inducible promoter-therapeutic payload construct is provided wherein the extracellular antigen binding domain comprises at least one single chain variable fragment of an antibody that binds to the antigen, or at least one heavy chain variable region of an antibody that binds to the antigen, or a combination thereof.

In another embodiment, a CAR-based surface antigen-regulated inducible promoter-therapeutic payload construct is provided wherein the at least one transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154, or a combination thereof.

In some embodiments, a CAR-based surface antigen-regulated inducible promoter-therapeutic payload construct is provided wherein CAR additionally encodes an extracellular antigen binding domain comprising CD19, CD20, CD22, CD19/22, ROR1, mesothelin, CD33, CD38, CD123 (IL3RA), CD138, BCMA (CD269), GPC2, GPC3, FGFR4, c-Met, PSMA, Glycolipid F77, EGFRvIII, GD-2, NY-ESO-1 TCR, MAGE A3 TCR, or an amino acid sequence with 85%, 90%, 95%, 96%, 97%, 98% or 99% identity thereof, or any combination thereof.

In one embodiment, a CAR-based surface antigen-regulated inducible promoter-therapeutic payload construct is provided wherein the extracellular antigen binding domain comprises an anti-CD19 ScFv antigen binding domain, an anti-CD20 ScFv antigen binding domain, an anti-CD22 ScFv antigen binding domain, an anti-ROR1 ScFv antigen binding domain, an anti-mesothelin ScFv antigen binding domain, an anti-CD33 ScFv antigen binding domain, an anti-CD38 ScFv antigen binding domain, an anti-CD123 (IL3RA) ScFv antigen binding domain, an anti-CD138 ScFv antigen binding domain, an anti-BCMA (CD269) ScFv antigen binding domain, an anti-GPC2 ScFv antigen binding domain, an anti-GPC3 ScFv antigen binding domain, an anti-FGFR4 ScFv antigen binding domain, an anti-c-Met ScFv antigen binding domain, an anti-PMSA ScFv antigen binding domain, an anti-glycolipid F77 ScFv antigen binding domain, an anti-EGFRvIII ScFv antigen binding domain, an anti-GD-2 ScFv antigen binding domain, an anti-NY-ESo-1 TCR ScFv antigen binding domain, an anti-MAGE A3 TCR ScFv antigen binding domain, or an amino acid sequence with 85%, 90%, 95%, 96%, 97%, 98% or 99% identity thereof, or any combination thereof.

In another embodiment, a CAR-based surface antigen-regulated inducible promoter-therapeutic payload construct is provided wherein the extracellular antigen binding domain comprises an immunoglobulin variable heavy chain only (VH) anti-CD19 antigen binding domain, an anti-CD20 VH antigen binding domain, an anti-CD22 VH antigen binding domain, an anti-ROR1 VH antigen binding domain, an anti-mesothelin VH antigen binding domain, an anti-CD33 VH antigen binding domain, an anti-CD38 VH antigen binding domain, an anti-CD123 (IL3RA) VH antigen binding domain, an anti-CD138 VH antigen binding domain, an anti-BCMA (CD269) VH antigen binding domain, an anti-GPC2 VH antigen binding domain, an anti-GPC3 VH antigen binding domain, an anti-FGFR4 VH antigen binding domain, an anti-c-Met VH antigen binding domain, an anti-PMSA VH antigen binding domain, an anti-glycolipid F77 VH antigen binding domain, an anti-EGFRvIII VH antigen binding domain, an anti-GD-2 VH antigen binding domain, an anti-NY-ESO-1 TCR VH antigen binding domain, an anti-MAGE A3 TCR VH antigen binding domain, or an amino acid sequence with 85%, 90%, 95%, 96%, 97%, 98% or 99% identity thereof, or any combination thereof.

In another embodiment, a CAR-based surface antigen-regulated inducible promoter-therapeutic payload construct is provided wherein the extracellular antigen binding domain comprises a protein or a peptide (P) sequence capable of specifically binding target antigen, which may be derived from a natural or a synthetic sequence comprising anti-CD19 P antigen binding domain, an anti-CD20 P antigen binding domain, an anti-CD22 P antigen binding domain, an anti-ROR1 P antigen binding domain, an anti-mesothelin P antigen binding domain, an anti-CD33 P antigen binding domain, an anti-CD38 P antigen binding domain, an anti-CD123 (IL3RA) P antigen binding domain, an anti-CD138 P antigen binding domain, an anti-BCMA (CD269) P antigen binding domain, an anti-GPC2 P antigen binding domain, an anti-GPC3 P antigen binding domain, an anti-FGFR4 P antigen binding domain, an anti-c-Met P antigen binding domain, an anti-PMSA P antigen binding domain, an anti-glycolipid F77 P antigen binding domain, an anti-EGFRvIII P antigen binding domain, an anti-GD-2 P antigen binding domain, an anti-NY-ESO-1 TCR P antigen binding domain, an anti-MAGE A3 TCR P antigen binding domain, or an amino acid sequence with 85%, 90%, 95%, 96%, 97%, 98% or 99% identity thereof, or any combination thereof. In another embodiment, a CAR is provided wherein the at least one intracellular signaling domain comprises a costimulatory domain and a primary signaling domain.

In yet another embodiment, a CAR-based surface antigen-regulated inducible promoter-therapeutic payload construct is provided wherein the at least one intracellular signaling domain comprises a costimulatory domain comprising a functional signaling domain of a protein selected from the group consisting of OX40, CD70, CD27, CD28, CD5, ICAM-1, LFA-1 (CD11a/CD18), ICOS (CD278), DAP10, DAP12, and 4-1BB (CD137), or a combination thereof.

In another embodiment, the nucleic acid sequence encoding a CAR-based surface antigen-regulated inducible promoter-therapeutic payload construct comprises the nucleic acid sequence of SEQ ID NO: 139. In one embodiment, the nucleic acid sequence encodes a CAR-based promoter-therapeutic payload construct comprising the amino acid sequence of SEQ ID NO: 78.

In another embodiment, the nucleic acid sequence encoding a CAR-based surface antigen-regulated inducible promoter-therapeutic payload construct comprises the nucleic acid sequence of SEQ ID NO: 140. In one embodiment, the nucleic acid sequence encodes a CAR-based promoter-therapeutic payload construct comprising the amino acid sequence of SEQ ID NO: 78.

In another embodiment, the nucleic acid sequence encoding a CAR-based surface antigen-regulated inducible promoter-therapeutic payload construct comprises the nucleic acid sequence of SEQ ID NO: 77. In one embodiment, the nucleic acid sequence encodes a CAR-based promoter-therapeutic payload construct comprising the amino acid sequence of SEQ ID NO: 78.

In one aspect, the surface antigen-regulated inducible promoter CAR-based therapeutic payload constructs disclosed herein are modified to express or contain a detectable marker for use in diagnosis, monitoring, and/or predicting the treatment outcome such as progression free survival of cancer patients or for monitoring the progress of such treatment.

In one embodiment, the nucleic acid molecule encoding the CAR-based surface antigen-regulated inducible promoter-therapeutic payload constructs can be contained in a vector, such as a viral vector. The vector is a DNA vector, an RNA vector, a plasmid vector, a cosmid vector, a herpes virus vector, a measles virus vector, a lentivirus vector, adenoviral vector, or a retrovirus vector, or a combination thereof.

In yet another embodiment, the vector expressing the CAR-based surface antigen-regulated inducible promoter-therapeutic payload constructs construct can be further modified to include one or more operative elements to control the expression of CAR T cells, or to eliminate CAR-T cells by virtue of a suicide switch. The suicide switch can include, for example, an apoptosis inducing signaling cascade or a drug that induces cell death. In a preferred embodiment, the vector expressing the CAR can be further modified to express an enzyme such thymidine kinase (TK) or cytosine deaminase (CD).

In another aspect, host cells including the nucleic acid molecule encoding the CAR-based surface antigen-regulated inducible promoter-therapeutic payload constructs are also provided. In some embodiments, the host cell is a T cell, such as a primary T cell obtained from a subject. In one embodiment, the host cell is a CD8⁺ T cell.

In one aspect, a transduced T cell comprising an isolated nucleic acid molecule encoding a therapeutic payload operably connected to a surface antigen-regulated inducible promoter is provided herein, wherein the surface antigen-regulated inducible promoter therapeutic payload construct confers upon said transduced CAR T cell the ability to mount an antitumor response dependent upon the level of expression of surface antigen on the target cell thereby achieving a T-cell response precisely regulated to the level of target antigen present in the tumor milieu.

In another aspect, a transduced CAR T cell comprising an isolated nucleic acid molecule encoding a CAR operably connected to a surface antigen-regulated inducible promoter is provided herein, wherein the surface antigen-regulated inducible promoter CAR construct confers upon said transduced CAR T cell the ability to mount an antitumor response dependent upon the level of expression of corresponding surface antigen on the target cell thereby achieving a CAR T-cell response precisely regulated to the level of target surface antigen present in the tumor milieu, wherein i) in the absence of tumor target surface antigen expression, surface antigen-regulated inducible promoter confers a low basal level of CAR expression; ii) in the presence of activating target surface antigen on the surface of a target cell, the CAR is activated which triggers activation of the applicable signal transduction pathway(s), thereby activating the signal mediator(s) of the surface antigen-regulated inducible promoter thereby leading to increased expression of the CAR above the basal level of expression; iii) higher expression of a given target surface antigen leads to higher expression of the CAR and vice versa thereby leading to efficient regulation of CAR expression to achieve a CAR T-cell response precisely tailored to the level of target present at the specific site and time; iv) heightened CAR expression leads to an optimal anti-tumor activity and rapid elimination of target tumor cells; and v) as tumor cells are reduced/removed in amount, the level of the CAR expression returns to its basal level of expression.

In one embodiment, a transduced T cell with heightened CAR expression leads to an optimal anti-tumor activity and rapid elimination of target tumor cells such that as tumor cells are reduced/removed in amount, the level of the therapeutic payload expression returns to the basal level of expression (c.f., the pre-antitumor response expression level).

In one embodiment the transduced T cells are autologous. In another embodiment the transduced T cells are allogeneic.

In yet another aspect, a pharmaceutical composition is provided comprising an anti-tumor effective amount of a population of human T cells, wherein the T cells comprise a nucleic acid sequence that encodes a CAR-based surface antigen-regulated inducible promoter-therapeutic payload constructs, wherein the CAR comprises at least one extracellular antigen binding domain comprising an antigen binding domain comprising the amino acid sequence selected from the group consisting of SEQ ID NOs: 8, 10, 12, 16, 18, 20, 22, 24, 26, 88, 90, 92, 94, 98, 116, 118, 120, 122, 124, 126, 128, 130, 132, 134, and 136; at least one linker domain; at least one transmembrane domain; and at least one intracellular signaling domain, wherein the T cells are T cells of a human having a cancer. The cancer includes, inter alia, a hematological cancer such as leukemia (e.g., chronic lymphocytic leukemia (CLL), acute lymphocytic leukemia (ALL), or chronic myelogenous leukemia (CML), lymphoma (e.g., mantle cell lymphoma, non-Hodgkin's lymphoma or Hodgkin's lymphoma) or multiple myeloma (MM), or a combination thereof.

In one embodiment, a pharmaceutical composition is provided wherein the at least one transmembrane domain of the CAR-based surface antigen-regulated inducible promoter-therapeutic payload constructs contains a transmembrane domain of a protein selected from the group consisting of the alpha, beta or zeta chain of the T-cell receptor, CD28, CD3 epsilon, CD45, CD4, CD5, CD8, CD9, CD16, CD22, Mesothelin, CD33, CD37, CD64, CD80, CD86, CD134, CD137 and CD154, or a combination thereof.

In another embodiment, a pharmaceutical composition is provided wherein the human cancer includes an adult carcinoma comprising oral and pharynx cancer (tongue, mouth, pharynx, head and neck), digestive system cancers (esophagus, stomach, small intestine, colon, rectum, anus, liver, interhepatic bile duct, gallbladder, pancreas), respiratory system cancers (larynx, lung and bronchus), bones and joint cancers, soft tissue cancers, skin cancers (melanoma, basal and squamous cell carcinoma), pediatric tumors (neuroblastoma, rhabdomyosarcoma, osteosarcoma, Ewing's sarcoma), tumors of the central nervous system (brain, astrocytoma, glioblastoma, glioma), and cancers of the breast, the genital system (uterine cervix, uterine corpus, ovary, vulva, vagina, prostate, testis, penis, endometrium), the urinary system (urinary bladder, kidney and renal pelvis, ureter), the eye and orbit, the endocrine system (thyroid), and the brain and other nervous system, or any combination thereof.

In yet another embodiment, a pharmaceutical composition is provided comprising an anti-tumor effective amount of a population of human T cells of a human having a cancer wherein the cancer is a refractory cancer non-responsive to one or more chemotherapeutic agents. The cancer includes hematopoietic cancer, myelodysplastic syndrome pancreatic cancer, head and neck cancer, cutaneous tumors, minimal residual disease (MRD) in acute lymphoblastic leukemia (ALL), acute myeloid leukemia (AML), adult B cell malignancies including, CLL (Chronic lymphocytic leukemia), CML (chronic myelogenous leukemia), non-Hodgkin's lymphoma (NHL), pediatric B cell malignancies (including B lineage ALL (acute lymphocytic leukemia)), multiple myeloma (MM), lung cancer, breast cancer, ovarian cancer, prostate cancer, colon cancer, melanoma or other hematological cancer and solid tumors, or any combination thereof.

In yet another embodiment, a pharmaceutical composition is provided wherein the surface antigen-regulated inducible promoter-therapeutic payload constructs adjust the activity of the therapeutic payload by utilizing native effector cell pathways based on a native cell activation, mitogenic state, or a combination thereof.

In another aspect, methods of making CAR-containing T cells (hereinafter “CAR-T cells”) are provided. The methods include transducing a T cell with a vector or nucleic acid molecule comprising a surface antigen-regulated inducible promoter-therapeutic payload encoding a disclosed CAR that specifically binds to mesothelin, CD33, CD19, CD19/CD20, CD22, CD19/22, ROR1, CD123, or CD38, or a combination thereof, thereby making the CAR-T cell.

In yet another aspect, a method of generating a population of RNA-engineered cells is provided that comprises introducing an in vitro transcribed RNA or synthetic RNA of a nucleic acid molecule comprising a surface antigen-regulated inducible promoter-therapeutic payload encoding a disclosed CAR into a cell of a subject, thereby generating a CAR T cell.

In yet another aspect, a method for diagnosing a disease, disorder or condition associated with the expression of a surface antigen-regulated inducible promoter-therapeutic payload construct in a cell comprising a nucleotide sequence selected from the group consisting of SEQ ID NOs: 139, 140, and 141, or a combination thereof, is provided comprising contacting the cell with a human anti-mesothelin, anti-CD33, anti-CD19, anti-CD19/CD20, anti-CD22, anti-ROR1, anti-CD123, or anti-CD38, antibody or fragment thereof, or a combination thereof, wherein the antibody or a fragment thereof comprises an amino acid sequence selected from the group consisting of SEQ ID NOs: 8, 10, 12, 16, 18, 20, 22, 24, 26, 88, 90, 92, 94, 98, 116, 118, 120, 122, 124, 126, 128, 130, 132, 134, and 136, or a combination thereof; and b) detecting the presence of the antigens mesothelin, CD33, CD19, CD19/CD20, CD22, ROR1, CD123, or CD38, or a combination thereof wherein the presence of mesothelin, CD33, CD19, CD19/CD20, CD22, ROR1, CD123, or CD38, or a combination thereof diagnoses for the disease, disorder or condition associated with the expression of mesothelin, CD33, CD19, CD19/CD20, CD22, ROR1, CD123, or CD38, or a combination thereof.

In one embodiment, the disease, disorder or condition associated with the expression of mesothelin, CD33, CD19, CD19/CD20, CD22, CD19/22, ROR1, CD123, or CD38, or a combination thereof, is cancer including hematopoietic cancer, myelodysplastic syndrome pancreatic cancer, head and neck cancer, cutaneous tumors, minimal residual disease (MRD) in acute lymphoblastic leukemia (ALL), acute myeloid leukemia (AML), adult B cell malignancies including, CLL (chronic lymphocytic leukemia), CML (chronic myelogenous leukemia), non-Hodgkin's lymphoma (NHL), pediatric B cell malignancies (including B lineage ALL (acute lymphocytic leukemia)), multiple myeloma (MM), lung cancer, breast cancer, ovarian cancer, prostate cancer, colon cancer, melanoma or other hematological cancer and solid tumors, or any combination thereof.

In another embodiment, a method of diagnosing, prognosing, or determining risk of a mesothelin-, CD33-, CD19-, CD19/CD20-, CD22-, CD19/22-, ROR1-, CD123-, or CD38- (or combination thereof) related disease in a mammal, is provided comprising detecting the expression of mesothelin, CD33, CD19, CD19/CD20, CD22, CD19/22, ROR1, CD123, or CD38, or a combination thereof, in a sample derived from the mammal comprising: a) contacting the sample with a surface antigen-regulated inducible promoter-therapeutic payload construct comprising human anti-mesothelin, anti-CD33, anti-CD19, anti-CD19/CD20, anti-CD22, anti-CD19/22, anti-ROR1, anti-CD123, or anti-CD38, (or a combination thereof) antibody or fragment thereof, wherein the antibody or a fragment thereof comprises an amino acid sequence selected from the group consisting of SEQ ID NOs: 8, 10, 12, 16, 18, 20, 22, 24, 26, 88, 90, 92, 94, 98, 116, 118, 120, 122, 124, 126, 128, 130, 132, 134, and 136; and b) detecting the presence of one or more antigens wherein the presence of said antigen diagnoses for a mesothelin, CD33-, CD19, CD19/CD20-, CD22-, CD19/22-, ROR1-, CD123-, or CD38-related disease in the mammal.

In another embodiment, a method of redirecting CAR antigen targets, is provided comprising contacting a cell with a surface antigen-regulated inducible promoter-therapeutic payload construct comprising human anti-mesothelin, anti-CD33, anti-CD19, anti-CD19/CD20, anti-CD22, anti-CD19/22, anti-ROR1, anti-CD123, or anti-CD38, antibody or fragment thereof, wherein the antibody or a fragment thereof comprises an amino acid sequence selected from the group consisting of SEQ ID NOs: 8, 10, 12, 16, 18, 20, 22, 24, 26, 88, 90, 92, 94, 98, 116, 118, 120, 122, 124, 126, 128, 130, 132, 134, and 136. In one embodiment, the cell is selected from the group consisting of a mesothelin, CD33, CD19, CD19/CD20, CD22, CD19/22, ROR1, CD123, or CD38-expressing tumor cell, a tumor-associated macrophage, and any combination thereof.

In another aspect, a method is provided for inducing an anti-tumor immunity in a mammal comprising administering to the mammal a therapeutically effective amount of a T cell transduced with a surface antigen-regulated inducible promoter-therapeutic payload construct comprising a vector or nucleic acid molecule encoding a disclosed therapeutic payload, for example, and not by way of limitation, a CAR, cytokine, chemokine, trafficking receptor, bispecific antibody, a neutralizing/blocking antibody, a T-cell stimulatory receptor, truncated inhibitory receptor, hybrid inhibitory/activating receptor, anti-apoptotic protein, shRNA, or protease, or a combination thereof.

In another embodiment, a method of treating or preventing cancer in a mammal is provided comprising administering to the mammal one or more of the disclosed surface antigen-regulated promoter-therapeutic payload constructs, in an amount effective to treat or prevent cancer in the mammal. The method includes administering to the subject a therapeutically effective amount of host cells expressing a disclosed CAR that specifically binds mesothelin, CD33, CD19, CD19/CD20, CD22, CD19/22, ROR1, CD123, or CD38 and/or one or more of the aforementioned antigens, under conditions sufficient to form an immune complex of the antigen binding domain on the CAR and the extracellular domain of mesothelin, CD33, CD19, CD19/CD20, CD22, CD19/22, ROR1, CD123, or CD38 and/or one or more of the aforementioned antigens in the subject.

In yet another embodiment, a method is provided for treating a mammal having a disease, disorder or condition associated with an elevated expression of a tumor antigen, the method comprising administering to the subject a pharmaceutical composition comprising one or more of the disclosed surface antigen-regulated inducible promoter-therapeutic payload constructs, comprising an anti-tumor effective amount of a population of T-cells, wherein the T-cells comprise a nucleic acid sequence that encodes a CAR, wherein the CAR includes at least one extracellular mesothelin, CD33, CD19, CD19/CD20, CD22, CD19/22, ROR1, CD123, or CD38 antigen binding domain comprising the amino acid sequence of SEQ ID NOs: 8, 10, 12, 16, 18, 20, 22, 24, 26, 88, 90, 92, 94, 98, 116, 118, 120, 122, 124, 126, 128, 130, 132, 134, and 136, or any combination thereof, at least one linker or spacer domain, at least one transmembrane domain, at least one intracellular signaling domain, and wherein the T-cells are T-cells of the subject having cancer.

In yet another embodiment, a method is provided for treating cancer in a subject in need thereof comprising administering to the subject a pharmaceutical composition comprising one or more of the disclosed surface antigen-regulated inducible promoter-therapeutic payload constructs, comprising an anti-tumor effective amount of a population of T-cells, wherein the T-cells comprise a nucleic acid sequence that encodes a chimeric antigen receptor (CAR), wherein the CAR comprises at least one mesothelin, CD33, CD19, CD19/CD20, CD22, CD19/22, ROR1, CD123, or CD38 antigen binding domain comprising the amino acid sequence of SEQ ID NOs: 8, 10, 12, 16, 18, 20, 22, 24, 26, 88, 90, 92, 94, 98, 116, 118, 120, 122, 124, 126, 128, 130, 132, 134, and 136, or any combination thereof, at least one linker or spacer domain, at least one transmembrane domain, at least one intracellular signaling domain, wherein the T-cells are T-cells of the subject having cancer. In some embodiments of the aforementioned methods, the at least one transmembrane domain comprises a transmembrane domain of the alpha, beta or zeta chain of the T-cell receptor, CD28, CD3 epsilon, CD45, CD4, CD5, CD8, CD9, CD16, CD22, Mesothelin, CD33, CD37, CD64, CD80, CD86, CD134, CD137 and CD154, or a combination thereof.

In yet another embodiment, a method is provided for generating a persisting population of genetically engineered T-cells in a human diagnosed with cancer. In one embodiment, the method comprises administering to a human a T-cell genetically engineered to express a surface antigen-regulated promoter-therapeutic payload construct (e.g. CAR-, cytokine-, chemokine-, trafficking receptor-, bispecific antibody-, a neutralizing/blocking antibody-, a T cell stimulatory receptor-, a truncated inhibitory receptor-, a hybrid inhibitory/activating receptor-, an anti-apoptotic protein-, an shRNA-, a protease-based) wherein the self-driving surface antigen-regulated promoter-therapeutic payload construct comprises at least one mesothelin, CD33, CD19, CD19/CD20, CD22, CD19/22, ROR1, CD123, or CD38 antigen binding domain comprising the amino acid sequence of SEQ ID NOs: 8, 10, 12, 16, 18, 20, 22, 24, 26, 88, 90, 92, 94, 98, 116, 118, 120, 122, 124, 126, 128, 130, 132, 134, and 136, or any combination thereof; at least one transmembrane domain; and at least one intracellular signaling domain wherein the persisting population of genetically engineered T-cells, or the population of progeny of the T-cells, persists in the human for at least one month, two months, three months, four months, five months, six months, seven months, eight months, nine months, ten months, eleven months, twelve months, two years, or three years after administration.

In one embodiment, the progeny T-cells in the human comprise a memory T-cell. In another embodiment, the T-cell is an autologous T-cell.

In all of the aspects and embodiments of methods described herein, any of the aforementioned cancers, diseases, disorders or conditions associated with an elevated expression of a tumor antigen that may be treated or prevented or ameliorated using one or more of the surface antigen-regulated promoter-therapeutic payload constructs disclosed herein.

For the avoidance of doubt, the specification and claims as disclosed herein specifically exclude by way of proviso the Syn-Notch constructs as set forth in U.S. Pat. No. 9,670,281, issued on Jun. 6, 2017 entitled Binding-triggered transcriptional switches and methods of use thereof and U.S. Pat. No. 9,834,608, issued on Dec. 5, 2017 (Wendell A. Lim et al.), respectively, and the NFAT-regulated expression of IL-12, as set forth in U.S. Pat. No. 8,556,882, issued on Oct. 15, 2013 (Richard A. Morgan et al.).

In yet another aspect, a kit is provided for making a surface antigen-regulated promoter-therapeutic payload construct in a T-cell as described supra or for preventing, treating, or ameliorating any of the cancers, diseases, disorders or conditions associated with an elevated expression of a tumor antigen in a subject as described supra, comprising a container comprising any one of the nucleic acid molecules, vectors, host cells, or compositions disclosed supra or any combination thereof, and instructions for using the kit.

It will be understood that the surface antigen-regulated promoter-therapeutic payload constructs, host cells, nucleic acids, and methods are useful beyond the specific aspects and embodiments that are described in detail herein. The foregoing features and advantages of the disclosure will become more apparent from the following detailed description, which proceeds with reference to the accompanying figures.

### BRIEF DESCRIPTION OF THE FIGURES

FIG. 1 depicts a Schematic Diagram of positive regulation of the self-driving STAT5 CAR via IL-2 pathway and AP1/NFκB CAR via cytokine/CAR stimulation pathways.

FIG. 2 depicts the structure of STAT5 and AP1/NFκB inducible CAR LTG1563, and constitutive EF1α-driven CAR LTG1563.

FIG. 3 depicts induction of CAR expression by cytokine—driven positive feedback loop in STAT5 and AP1/NFκB inducible CAR LTG1563 in one healthy donor (representative of 2 donors). T cells were treated with varying concentrations of IL2 and TNFα for 18 h, 5 days post-activation (4 days post-transduction).

FIG. 4 depicts AP1/NFκB-regulated self-driving CAR LTG1563 exhibits rapid recovery of CAR expression following antigen exposure, as compared to the EF1α-driven constitutive CAR. The expression in self-driving CAR constructs regulated by the inducible STAT5 & AP1/NFκB promoters, and in EF1α-driven constitutive CAR construct, following initial coculture with Raji cells in 2 healthy donors. Cells were activated with TransAct reagent at DO, transduced at D1 with LV vectors at an MOI of 10, and washed and treated with either: 0 IU/mL IL-2 or 30 IU/mL IL2 from D2-D6 post-activation. From D6 post-activation, CAR LTG1563 T cells were cocultured with Raji-GFP (CD19+) cells at an effector:target ratio of 1:3. CAR expression prior to coculture (D6) and 1 day after coculture (D7) is shown, one representative donor of 2.

FIGS. 5A-5C depict superior Raji tumor killing and T cell expansion with CAR LTG1563 T cells regulated by AP1-NFκB-positive feedback loop (FIG. 5A) Long-term killing assay: Coculture time course diagram. Cells were activated with TransAct reagent at DO, transduced at D1 with LV vectors at an MOI of 10, and washed and treated with either: 0 IU/mL IL-2 or 30 IU/mL IL2 from D2-D6 post-activation. From D6-D13 post-activation, CAR LTG1563 T cells were cocultured with Raji-GFP (CD19+) cells at an effector:target ratio of 1:3 (Coculture 1), and from D13-D20, cells were re-stimulated with Raji-GFP cells. (FIG. 5B) CAR LTG1563-dependent Raji cytotoxicity during Coculture 1. (FIG. 5C) CAR LTG1563-dependent T cell expansion during Cocultures 1&2. (FIG. 5B, FIG. 5C). n=3 donors (*: p<0.05, **:p<0.01, ***:p<0.001, ****:p<0.0001; 2-way ANOVA with Tukey's correction).

FIG. 6 depicts the structure of AP1/NFκB inducible and constitutive EF1α-driven CARs with additional auxiliary components (for example, dominant negative receptors): CAR LTG1563-TGFBRIIdn, CAR LTG1563-PD1dn, and CAR LTG1563-PD1dn-TGFBRIIdn.

FIGS. 7A-C depict AP1/NFκB-regulated self-driving CAR LTG 1563 with additional auxiliary components, which exhibits rapid induction of the payload (CAR and the associated auxiliary components: TGFBRIIdn, PD1dn) expression following antigen exposure, as compared to the EF1α-driven constitutive CAR. The expression in self-driving CAR constructs regulated by the inducible AP1/NFκB promoters, and in EF1α-driven constitutive CAR construct, following initial coculture with Raji cells in 2 healthy donors. Cells were activated with TransAct reagent at DO, transduced at D1 with LV vectors at an MOI of 10, and washed and treated with 30 IU/mL IL2 from D2-D5 post-activation. From D5 post-activation, CAR LTG1563 T cells were cocultured with Raji-GFP (CD19+) cells at an effector:targetratio of 1:3. CAR, TGFBRII, and PD1 expression prior to coculture (D5) and 1 day after coculture (D6) are shown, one representative donor of 2.

FIGS. 8A-8C depict superior Raji tumor killing and T cell expansion with CAR LTG 1563 (with additional auxiliary components) T cells protected from TGF-β suppression. (FIG. 8A) Coculture time course diagram of a long-term killing assay. Cells were activated with TransAct reagent at DO, transduced at D1 with LV vectors (0.5% v/v), and washed and treated with either: 0 IU/mL IL-2 or 30 IU/mL IL2 from D2-D5 post-activation. From D5-D9 post-activation, CAR LTG1563 T cells were cocultured with Raji-GFP (CD19+) cells at an effector:target ratio of 1:3 in the presence or absence of 10 ng/mL TGF-β (Coculture 1); from D9-D13, cells were re-stimulated with Raji-GFP cells in the presence or absence of 10 ng/mL TGF-β (Coculture 2), and from D13-19, cells were re-stimulated with Raji-GFP cells in the presence or absence of 10 ng/mL TGF-β (Coculture 3). (FIG. 8B) CAR LTG1563-dependent T cell expansion during Cocultures 1-3. (FIG. 8C) CAR LTG1563-dependent Raji cytotoxicity during Cocultures 1-3. (FIG. 8B, FIG. 8C). n=2 donors.

FIGS. 9A-C depict superior Raji tumor killing and less T cell exhaustion with CAR LTG 1563 (with additional auxiliary components) T cells protected from TGF-β suppression. (FIG. 9A) Coculture time course diagram of a long-term killing assay. Cells were activated with TransAct reagent at DO, transduced at D1 with LV vectors (0.5% v/v), and washed and treated with 30 IU/mL IL2 from DO-D8 post-activation. From D8-D14 post-activation, CAR LTG1563 T cells were cocultured with Raji-GFP (CD19+) cells at an effector:target ratio of 1:3 in the presence or absence of 10 ng/mL TGF-β (Coculture 1); from D14-D20, cells were re-stimulated with Raji-GFP cells at an effector:target ratio of 1:3 in the presence or absence of 10 ng/mL TGF-β (Coculture 2). (FIG. 9B) CAR LTG1563-dependent Raji cytotoxicity during Cocultures 1-2. (FIG. 9C) Quantification of CAR T exhaustion marker expression (PD1) at D20 post-activation (Coculture 2, D8) by flow cytometry at various time points pre- and post-coculture with CD19+ Raji-GFP cells (Cocultures 1&2). (FIG. 9B,FIG. 9C). *: p<0.05, **: p<0.01, ***:p<0.001, ****:p<0.0001. UTD=untransduced T cells. n=3 independent donors.

FIGS. 10A-E depicts superior Raji tumor killing, increased T cell expansion, and increased T cell cytokine production with CAR LTG 1563 (with additional auxiliary components) T cells protected from both TGF-β and PD-L1 suppression. (FIG. 10A) Expression of PD-L1 on transduced and magnetically-sorted Raji-GFP-PDL1 NHL B cell lines. (FIG. 10B) Coculture time course diagram for the long-term killing assay. Cells were activated with TransAct reagent at DO, transduced at D1 with LV vectors (0.5% v/v), and washed and treated with 30 IU/mL IL2 from DO-D8 post-activation. From D8-D14 post-activation, CAR LTG1563 T cells were cocultured with Raji-GFP (CD19+, PDL1−) or Raji-GFP-PDL1 (CD19+, PDL1+) cells at an effector:target ratio of 1:3 in the presence or absence of 10 ng/mL TGF-β (Coculture 1); from D14-D20, cells were re-stimulated with Raji-GFP or Raji-GFP-PDL1 cells at an effector:target ratio of 1:3 in the presence or absence of 10 ng/mL TGF-β (Coculture 2). (FIG. 10C) CAR LTG1563-dependent Raji cytotoxicity at the final timepoint analyzed in Coculture 1 (D14 post-activation) and Coculture 2 (D20 post-activation). (FIG. 10D) CAR LTG1563-dependent T cell expansion during full T cell culture period, including Cocultures 1-2. (FIG. 10E) Quantification of CAR T IL-2 production by flow cytometry 24 hours post-coculture with CD19+ Raji-GFP or Raji-GFP-PDL1 cells (Cocultures 1&2). (FIGS. 10B-E). *: p<0.05, **: p<0.01, ***:p<0.001, ****:p<0.0001. UTD=untransduced T cells. n=3 independent donors.

### DETAILED DESCRIPTION

### Definitions

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” Thus, “comprising an antigen” means “including an antigen” without excluding other elements. The phrase “and/or” means “and” or “or.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the Materials, Methods, and Examples are illustrative only and not intended to be limiting. To facilitate review of the various embodiments, the following explanations of terms are provided:

The term “about” when referring to a measurable value such as an amount, a temporal duration, and the like, is meant to encompass variations of .+−.20% or in some instances .+−.10%, or in some instances .+−.5%, or in some instances .+−.1%, or in some instances .+−.0.1% from the specified value, as such variations are appropriate to perform the disclosed methods.

Unless otherwise noted, the technical terms herein are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: A Comprehensive Desk Reference, published by VCH Publishers, Inc., 1995; and other similar references.

The present invention as disclosed and described herein is based upon the unexpected discovery that modulation or adjustment of a surface antigen-regulated promoter's expression of a therapeutic payload construct can be directly correlated with the activity of the therapeutic payload, and therefore the level of expression of surface antigen in a target cell's milieu.

The present disclosure provides for novel self-driving inducible promoter-therapeutic payload constructs comprising one or more therapeutic payloads operably connected to a surface antigen-regulated inducible promoter, which surface antigen-regulated inducible promoter adjusts the level of expression of the one or more therapeutic payloads, dependent upon the level of expression of surface antigen in a target cell's milieu, as well as host cells (e.g., T-cells) expressing the surface antigen-regulated inducible promoter therapeutic payload constructs, and nucleic acid molecules encoding the surface antigen-regulated inducible promoter-therapeutic payload constructs.

Without being limited to any particular mechanism of action, as used herein, a “self-driving” surface antigen-regulated inducible promoter refers to the utilization of a surface antigen-regulated inducible promoter to drive the therapeutic payloads to provide for a low basal level of the therapeutic payload surface expression in the absence of tumor target antigen expression. In the presence of activating target antigen on the surface of a target cell, the therapeutic payload is activated which triggers activation of the applicable signal transduction pathway(s), thereby activating the signal mediator(s) of the surface antigen-regulated inducible promoter thereby leading to increased expression of the therapeutic payload above the basal level of expression. In this manner, a positive feed-back loop is created whereby higher expression of a given target antigen leads to higher expression of the therapeutic payload and vice versa thereby leading to efficient regulation of therapeutic payload expression to achieve a T-cell response precisely tailored to the level of target present at the specific site and time. Heightened therapeutic payload expression leads to an optimal anti-tumor activity and rapid elimination of target tumor cells. As tumor cells are reduced/removed in amount, the level of the therapeutic payload expression returns to its basal level of expression.

This self-driving mode of activation will be repeated upon a subsequent re-exposure to antigen (for example and not by way of limitation, that occurring through a relapse of the tumor, a metastatic event, tumor migration/spreading, etc.). As a result, this control over the timing and magnitude of the self-driving therapeutic payload T-cell response affords improved treatment efficacy, reduced risk of tumor escape, and reduced toxicity of the treatment. This is akin to biologically or endogenously tailoring the treatment dose to the state of disease at the cellular level.

What follows is a detailed description of the self-driving surface antigen-regulated promoter-therapeutic payload constructs including a description of the surface antigen-regulated inducible promoters, the therapeutic payloads, along with a more detailed description of the self-driving CAR-based surface antigen-regulated promoter-therapeutic payload constructs, antibodies and antigen binding fragments thereof, conjugates, nucleotides, expression, vectors, and host cells, methods of treatment, compositions, and kits employing the disclosed self-driving CAR-based surface antigen-regulated promoter-therapeutic payload constructs.

### A. Surface Antigen-Regulated Inducible Promoters

In one aspect, an isolated nucleic acid molecule is provided herein encoding a therapeutic payload operably connected to a surface antigen-regulated inducible promoter, and wherein said surface antigen-regulated inducible promoter adjusts the level of expression of the one or more therapeutic payloads, dependent upon the level of expression of surface antigen on the target cell thereby achieving a T-cell response precisely regulated to the level of target antigen present in the tumor milieu.

In one embodiment, an isolated nucleic acid molecule is provided herein encoding a therapeutic payload operably connected to a surface antigen-regulated inducible promoter comprising a nucleotide sequence comprising SEQ ID NO: 137 and 138, or a combination thereof, and wherein said surface antigen-regulated inducible promoter adjusts the level of expression of the one or more therapeutic payloads, dependent upon the level of expression of surface antigen on the target cell thereby achieving a T-cell response precisely regulated to the level of target antigen present in the tumor milieu.

In one embodiment, an isolated nucleic acid molecule is provided herein encoding a therapeutic payload operably connected to a surface antigen-regulated inducible promoter comprising a nucleotide sequence comprising SEQ ID NO: 137 and 138, or a combination thereof, or a sequence with 85%, 90%, 95%, 96%, 97%, 98% or 99% identity thereof, and wherein said surface antigen-regulated inducible promoter adjusts the level of expression of the one or more therapeutic payloads dependent upon the level of expression of surface antigen on the target cell thereby achieving a T-cell response precisely regulated to the level of target antigen present in the tumor milieu.

In another embodiment, an isolated nucleic acid molecule is provided herein encoding a therapeutic payload operably connected to a surface antigen-regulated inducible promoter comprising a nucleotide sequence comprising SEQ ID NO: 137 and 138, or a combination thereof, and wherein said surface antigen-regulated inducible promoter upregulates the level of expression of the one or more therapeutic payloads, dependent upon the level of expression of surface antigen on the target cell thereby achieving a T-cell response precisely regulated to the level of target antigen present in the tumor milieu.

In another embodiment, an isolated nucleic acid molecule is provided herein encoding a therapeutic payload operably connected to a surface antigen-regulated inducible promoter comprising a nucleotide sequence comprising SEQ ID NO: 137 and 138, or a combination thereof, or a sequence with 85%, 90%, 95%, 96%, 97%, 98% or 99% identity thereof, and wherein said surface antigen-regulated inducible promoter upregulates the level of expression of the one or more therapeutic payloads, dependent upon the level of expression of surface antigen on the target cell thereby achieving a T-cell response precisely regulated to the level of target antigen present in the tumor milieu.

In yet another embodiment, the level of expression of the surface antigen-regulated inducible promoter therapeutic payload constructs described herein may be upregulated for example, and not by way of limitation, from approximately 10-100%, 200%, 300%, 400%, and 500%. The ranges recited herein specifically includes all integer amounts therein as if specifically recited thereto.

In one embodiment, an isolated nucleic acid molecule is provided herein encoding a therapeutic payload operably connected to a surface antigen-regulated inducible promoter comprising a nucleotide sequence comprising SEQ ID NO: 137 and 138, or a combination thereof, and wherein said surface antigen-regulated inducible promoter downregulates the level of expression of the one or more therapeutic payloads, dependent upon the level of expression of surface antigen on the target cell thereby achieving a T-cell response precisely regulated to the level of target antigen present in the tumor milieu.

In another embodiment, an isolated nucleic acid molecule is provided herein encoding a therapeutic payload operably connected to a surface antigen-regulated inducible promoter comprising a nucleotide sequence comprising SEQ ID NO: 137 and 138, or a combination thereof, or a sequence with 85%, 90%, 95%, 96%, 97%, 98% or 99% identity thereof, and wherein said surface antigen-regulated inducible promoter downregulates the level of expression of the one or more therapeutic payloads, dependent upon the level of expression of surface antigen on the target cell thereby achieving a T-cell response precisely regulated to the level of target antigen present in the tumor milieu.

In yet another embodiment, the level of expression of the surface antigen-regulated inducible promoter therapeutic payload constructs described herein may be downregulated for example, and not by way of limitation, from approximately 10-100%, 200%, 300%, 400%, and 500%. The range recited herein specifically includes all integer amounts therein as if specifically recited thereto.

### B. Therapeutic Payloads

In its broadest aspect, self-driving surface antigen-regulated inducible promoter-therapeutic payload constructs comprising at least one therapeutic payload comprising chimeric antigen receptors (CARs), cytokines, chemokines, trafficking receptors, bispecific antibodies, neutralizing/blocking antibodies, T-cell stimulatory receptors, truncated inhibitory receptors, hybrid inhibitory/activating receptors, anti-apoptotic proteins, shRNA, or protease, or a combination thereof, operably connected to a surface antigen-regulated inducible promoter comprising a STAT5 response element, AP-1 response element, or NF kappa B response element, or a combination thereof, which surface antigen-regulated inducible promoter adjusts the level of expression of the therapeutic payload, dependent upon the level of expression of surface antigen on the target cell are provided herein, as well as host cells (e.g., T-cells) expressing the surface antigen-regulated inducible promoter therapeutic payload constructs, and nucleic acid molecules encoding the surface antigen-regulated inducible promoter-therapeutic payload constructs.

In one aspect, self-driving surface antigen-regulated inducible promoter-therapeutic payload constructs comprising a therapeutic CAR and at least one therapeutic payload comprising a CAR, cytokines, chemokines, trafficking receptors, bispecific antibodies, neutralizing/blocking antibodies, T-cell stimulatory receptors, truncated inhibitory receptors, hybrid inhibitory/activating receptors, anti-apoptotic proteins, shRNA, or protease, or a combination thereof, operably connected to a surface antigen-regulated inducible promoter comprising a STAT5 response element, AP-1 response element, or NF kappa B response element, or a combination thereof, which surface antigen-regulated inducible promoter adjusts the level of expression of the therapeutic CAR and therapeutic payload, dependent upon the level of expression of surface antigen on the target cell are provided herein, as well as host cells (e.g., T-cells) expressing the surface antigen-regulated inducible promoter therapeutic payload constructs, and nucleic acid molecules encoding the surface antigen-regulated inducible promoter-therapeutic payload constructs.

In one embodiment, an isolated nucleic acid molecule is provided wherein the surface antigen-regulated inducible promoter-therapeutic payload construct further comprises a cytokine comprising IL-2, IL-15, IL-7, TNFa, IFN gamma, IFN beta, IFN alpha, IL-21, IL-33, IL-22, IL-6, IL-10, IL-9, IL-4, IL-12, TGF beta, IL-17, IL-18, or any combination thereof.

In another embodiment, an isolated nucleic acid molecule is provided wherein the surface antigen-regulated inducible promoter-therapeutic payload construct further comprises for example and not by way of limitation, a chemokine comprising, CCL2, CCL3, CCL4, CCL5, CCL19, CCL21, CCL25, CXCL9, CXCL10, CXCL11, CXCL12, CXCL13, CXCL16, or any combination thereof.

In another embodiment, an isolated nucleic acid molecule is provided wherein the surface antigen-regulated inducible promoter-therapeutic payload construct further comprises a trafficking receptor, such as a cytokine receptor, for example, and not by way of limitation, CCR2, CCR3, CCR4, CCR7, CCR8, CCR9, CXCR3, CXCR4, CXCR6, SIP₁ or any combination thereof, which trafficking receptor serves to aid in the trafficking of CAR T cells to the tumor site.

In another embodiment, an isolated nucleic acid molecule is provided wherein the surface antigen-regulated inducible promoter-therapeutic payload construct further comprises for example and not by way of limitation, a bispecific antibody, including a Bi-specific T cell engager (BiTE), for example, and not by way of limitation, anti-CD3 and anti CD19 targeting, or anti-CD3 and anti CD22 targeting, or anti-CD3 and anti CD20 targeting, or anti-CD3 and anti CD33 targeting, or anti-CD3 and anti CD123 targeting, or anti-CD3 and anti CD38 targeting, other multi-targeting antibody.

In another embodiment, an isolated nucleic acid molecule is provided wherein the surface antigen-regulated inducible promoter-therapeutic payload construct further comprises a neutralizing/blocking antibody, for example, and not by way of limitation, against PD-L1, PD-L2, CD95L, TRAIL receptor, IL-6R, IL-1R, TGF beta receptor, PD-1, LAG-3, Tim-3, TGF beta, IL-10, CTLA-4, VISTA, TIGIT, IL-1, IL-1R, expressed as an scFv, or as IgG, or scFvFc, or VHH, or F(ab), or F(ab)2, or a native ligand binding domain, or in other configurations, which neutralizing/blocking antibody serves to potentiate T cell lytic function, cytokine release, persistence, proliferation potential, prevent T cell checkpoint blockade, exhaustion, apoptosis, activation-induced cell death.

In another embodiment, an isolated nucleic acid molecule is provided wherein the surface antigen-regulated inducible promoter-therapeutic payload construct further comprises a T cell stimulatory receptor, for example, and not by way of limitation, IL-2R alpha, IL-15R alpha, IL-7R alpha, CXCR5, which stimulatory receptor serves to potentiate T cell anti-tumor function.

In another embodiment, an isolated nucleic acid molecule is provided wherein the surface antigen-regulated inducible promoter-therapeutic payload construct further comprises a truncated inhibitory receptor (dominant negative; “dn”), for example, and not by way of limitation, dn-TGF beta receptor II, dn-PD-1, dn-CTLA-4, dn-IL-10 receptor, dn-KLRG1, dn-CD160, dn-TIM3, dn-LAG3, dn-BTLA, dn-VISTA, which truncated inhibitory receptor serves to prevent inhibition of T cell function.

In another embodiment, an isolated nucleic acid molecule is provided wherein the surface antigen-regulated inducible promoter-therapeutic payload construct further comprises a hybrid inhibitory/activating receptor, for example, and not by way of limitation, ectodomain of PD-1 fused to endodomain of CD28, ectodomain of TGF beta receptor II fused to endodomain of gp130, ectodomain of IL-10 receptor fused to endodomain of 4-1BB, or IL-4 receptor fused to an endo-domain of IL-7 receptor, which hybrid inhibitory/activating receptor serves to transform T cell-inhibitory signal into T-cell activating signal.

In another embodiment, an isolated nucleic acid molecule is provided wherein the surface antigen-regulated inducible promoter-therapeutic payload construct further comprises an anti-apoptotic protein, for example, and not by way of limitation, BCL-2, MCL-1, CED9, Bfl-1, Brag-1, A-1, or BCL-XL, which anti-apoptotic protein serves to extend T cell persistence and prevent apoptosis.

In another embodiment, an isolated nucleic acid molecule is provided wherein the surface antigen-regulated inducible promoter-therapeutic payload construct further comprises an shRNA, for example, and not by way of limitation, for PD-1, CTLA-4, KLRG-1, CD160, TGF beta receptor II, IL-10R, which shRNA serves to downregulate T cell-inhibitory factors and potentiate T cell anti-tumor function.

In another embodiment, an isolated nucleic acid molecule is provided wherein the surface antigen-regulated inducible promoter-therapeutic payload construct further comprises a protease, such as MMP2, MMP4, which protease serves to digest tumor stroma and increase T cell penetration into tumor.

In another embodiment, an isolated nucleic acid molecule is provided wherein the surface antigen-regulated inducible promoter-therapeutic payload construct further comprises a peptide, such as iRGD peptide, which peptide serves to increase tumor penetration of anti-cancer drugs.

In another embodiment, an isolated nucleic acid molecule is provided wherein the surface antigen-regulated inducible promoter-therapeutic payload construct further comprises a second CAR T construct, which CAR T construct serves to target a second tumor antigen, or target an antigen expressed on the suppressor cells present in the tumor microenvironment, for example, and not by way of limitation, PD-L1, PD-L2, TRAIL receptor CD33, CD138, present on MDSC and inhibitory B cells.

In yet another embodiment, the one or more therapeutic payloads (for example, and not by way of limitation, chimeric antigen receptors (CARs), cytokines, chemokines, trafficking receptors, bispecific antibodies, neutralizing/blocking antibodies, T-cell stimulatory receptors, truncated inhibitory receptors, hybrid inhibitory/activating receptors, anti-apoptotic proteins, shRNA, or protease-based) are expressed under the control of the surface antigen-regulated inducible promoter, with separation of the one or more therapeutic payloads by a 2A ribosomal skip sequence or an internal ribosome entry sequence (IRES) or a combination thereof.

In yet another embodiment, an isolated nucleic acid molecule is provided wherein the surface antigen-regulated inducible promoter-therapeutic payload construct further comprises a cytokine comprising IL-2, IL-15, IL-7, TNFa, IFN gamma, IFN beta, IFN alpha, IL-21, IL-33, IL-22, IL-6, IL-10, IL-9, IL-4, IL-12, TGF beta, IL-17, IL-18, a chemokine comprising CCR4, CCR6, CXCR5, a trafficking receptor, such as a cytokine receptor, for example, and not by way of limitation, CCR4, CCR7, CCR2, a bispecific antibody, including a Bi-specific T cell engager (BiTE), for example, and not by way of limitation, anti-CD3 and anti CD19 targeting, or other multi-targeting antibody, a neutralizing/blocking antibody, for example, and not by way of limitation, against PD-L1, IL-6R, IL-1R, expressed as an scFv, or as IgG, or in other configurations, a T cell stimulatory receptor, a truncated inhibitory receptor, a hybrid inhibitory/activating receptor, for example, and not by way of limitation, ectodomain of PD-1, fused to endodomain of CD28, an anti-apoptotic protein, for example, and not by way of limitation, BCL-2, or BCL-XL, an shRNA, a protease, a second CAR T construct, or any combination thereof, each with their aforementioned biological properties as listed supra.

### C. Chimeric Antigen Receptors (CARs)

In a narrower aspect, the present invention as disclosed and described herein is based upon the unexpected discovery that modulation or adjustment of a surface antigen-regulated promoter's expression of a therapeutic payload construct can be directly correlated with the activity of the therapeutic payload, and therefore the level of expression of surface antigen in a target cell's milieu.

In contrast to previously-existing CAR T constructs for which the expression is regulated by constitutive promoters, the CAR-based surface antigen-regulated inducible promoter-therapeutic payload constructs described herein possess certain advantages over the prior art, including, for example, and not by way of limitation: i) adjusting or tailoring the timing and magnitude of the anti-tumor response to the specific amount of the antigen expressed at that time by the tumor, allowing optimal execution of anti-tumor CAR functions; ii) preventing detrimental T cell over-activation (exhaustion, activation induced cell death, reduced metabolic capacity, rapid terminal differentiation); iii) reducing or eliminating the risk of toxicities associated with inappropriate CAR activation or overactivation; iv) reducing or eliminating CAR-associated cytokine release syndrome (CRS); or v) reducing or eliminating CAR-associated neurotoxicity; or any combination thereof.

Thus, in one aspect of the invention as disclosed and described herein, the at least one CAR-based therapeutic payload construct self-driven by the surface antigen-regulated inducible promoter provided herein comprises at least one chimeric antigen receptor operably connected to a surface antigen-regulated inducible promoter, which surface antigen-regulated inducible promoter adjusts the level of expression of the one or more therapeutic payloads, dependent upon the level of expression of surface antigen on the target cell.

In one embodiment, the self-driving CAR-based surface antigen-regulated promoter-therapeutic payload constructs provided herein comprise, for example and not by way of limitation, one or more CAR-based therapeutic payloads operably connected to a surface antigen-regulated inducible promoter, and wherein the CAR-based therapeutic payload comprises a CAR comprising, from N-terminus to C-terminus, at least one extracellular binding domain comprising a mesothelin, CD33, CD19, CD19/CD20, CD22, CD19/22, ROR1, CD123, or CD38 antigen binding domain, or a combination thereof; at least one transmembrane domain, and at least one intracellular signaling domain.

A CAR is an artificially constructed hybrid protein or polypeptide containing the antigen binding domains of an antibody (e.g., single chain variable fragment (ScFv)) linked to T-cell signaling domains via the transmembrane domain. Characteristics of CARs include their ability to redirect T-cell specificity and reactivity toward a selected target in a non-MHC-restricted manner, and exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

The unique ability to combine functional moieties derived from different protein domains has been a key innovative feature of CARs. The choice of each of these protein domains is a key design feature, as is the way in which they are specifically combined. Each design domain is an essential component that can be used across different CAR platforms to engineer the function of lymphocytes. For example, the choice of the extracellular binding domain can make an otherwise ineffective CAR be effective.

The physico-chemical properties of the immunoglobulin-derived protein sequences used to create the extracellular antigen binding domain of a self-driving CAR-based surface antigen-regulated promoter-therapeutic payload construct can either be entirely neutral, or they can self-associate and drive the T-cell to a state of metabolic exhaustion, thus making the therapeutic T-cell expressing that self-driving CAR-based surface antigen-regulated promoter-therapeutic payload construct far less effective. This occurs independently of the antigen binding function of this CAR domain. Furthermore, the choice of the intracellular signaling domain(s) also can govern the activity and the durability of the therapeutic lymphocyte population used for immunotherapy. While the ability to bind target antigen and the ability to transmit an activation signal to the T-cell through these extracellular and intracellular domains, respectively, are important CAR design aspects, what has also become apparent is that the choice of the source of the extracellular antigen binding fragments can have a significant effect on the efficacy of the self-driving CAR-based surface antigen-regulated promoter-therapeutic payload construct and thereby have a defining role for the function and clinical utility of the self-driving CAR-based surface antigen-regulated promoter-therapeutic payload construct.

As disclosed herein, the intracellular T-cell signaling domains of the self-driving CAR-based surface antigen-regulated promoter-therapeutic payload constructs can include, for example, a T-cell receptor signaling domain, a T-cell costimulatory signaling domain, or both. The T-cell receptor signaling domain refers to a portion of the surface antigen-regulated promoter-therapeutic payload construct comprising the intracellular domain of a T-cell receptor, such as, for example, and not by way of limitation, the intracellular portion of the CD3 zeta protein. The costimulatory signaling domain refers to a portion of surface antigen-regulated promoter-therapeutic payload construct comprising the intracellular domain of a costimulatory molecule, which is a cell surface molecule other than an antigen receptor or their ligands that are required for an efficient response of lymphocytes to antigen.

What follows is a detailed description of the inventive self-driving CAR-based surface antigen-regulated promoter-therapeutic payload constructs including a description of their extracellular antigen binding domains, the transmembrane domain and the intracellular domain, along with additional description of the self-driving CAR-based surface antigen-regulated promoter-therapeutic payload constructs, antibodies and antigen binding fragments thereof, conjugates, nucleotides, expression, vectors, and host cells, methods of treatment, compositions, and kits employing the disclosed self-driving CAR-based surface antigen-regulated promoter-therapeutic payload constructs.

### 1. Extracellular Domain

In one embodiment, the CAR-based surface antigen-regulated promoter-therapeutic payload construct comprises a target-specific binding element otherwise referred to as an antigen binding domain or moiety. The choice of domain depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain of the therapeutic surface antigen target specific promoter-payload construct include those associated with viral, bacterial and parasitic infections, autoimmune disease and cancer cells.

In one embodiment, the surface antigen-regulated promoter-therapeutic payload construct can be engineered to target a tumor antigen of interest by way of engineering a desired antigen binding domain that specifically binds to an antigen on a tumor cell. Tumor antigens are proteins that are produced by tumor cells that elicit an immune response, particularly T-cell mediated immune responses. The selection of the antigen binding domain will depend on the particular type of cancer to be treated. Tumor antigens are well known in the art and include, for example, a glioma-associated antigen, carcinoembryonic antigen (CEA), .beta.-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. The tumor antigens disclosed herein are merely included by way of example. The list is not intended to be exclusive and further examples will be readily apparent to those of skill in the art.

In one embodiment, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSAs or TAAs include the following: Differentiation antigens such as MART-1/MelanA (MART-I), gp100 (Pmel 17), tyrosinase, TRP-1, TRP-2 and tumor-specific multi-lineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAGi6, TA-90\Mac-2 binding protein\cyclophilin C-associated protein, TAAL6, TAG72, TLP, and TPS.

Moreover, in certain embodiments, the use of a human extracellular antigen binding domain, instead of a mouse-derived binding domain, results in generation of a self-driving CAR-based surface antigen-regulated promoter-therapeutic payload construct that functions better in vivo, while avoiding the induction of anti-CAR immunity in the host immune response and the killing of the CAR-T cell population that is associated with murine-based antigen binding domains.

The self-driving CAR-based surface antigen-regulated promoter-therapeutic payload constructs expressing the entirely human extracellular ScFv antigen binding domain exhibit superior activities/properties including i) prevention of poor CAR-T persistence and function as seen with mouse-derived binding sequences; ii) lack of requirement for regional delivery of the self-driving CAR-based surface antigen-regulated promoter-therapeutic payload construct to be efficacious; and iii) ability to generate CAR-T cell designs based both on binders with high and low affinity to a respective antigen. This latter property allows investigators to better tune efficacy versus toxicity, and/or tissue specificity of the CAR-T product, since lower-affinity binders may have higher specificity to tumors versus normal tissues due to higher expression of certain antigens on tumors than normal tissue, which may prevent on-target off tumor toxicity and bystander cell killing.

In a preferred embodiment, the antigen binding domain portion of the CAR-based surface antigen-regulated promoter-therapeutic payload construct targets an antigen that includes but is not limited to CD19, CD20, CD22, ROR1, Mesothelin, CD33, c-Met, PSMA, Glycolipid F77, EGFRvIII, GD-2, MY-ESO-1 TCR, MAGE A3 TCR, and the like.

In one embodiment, the extracellular antigen binding domain in the CAR-based surface antigen-regulated promoter-therapeutic payload constructs may comprise, for example, the scFV binders as disclosed in Applicant's issued U.S. Pat. No. 10,183,993, entitled Compositions and Methods for Treating Cancer with Anti-Mesothelin Immunotherapy, first filed as Provisional Patent Application No. 62/444,201 on Jan. 9, 2017, and issued on Jan. 22, 2019, and assigned Lentigen Technology, Inc. matter number LEN_017.

In one preferred embodiment, the isolated nucleic acid molecule encoding the extracellular mesothelin antigen binding domain comprises the nucleotide sequence of SEQ ID NO: 87, or a sequence with 85%, 90%, 95%, 96%, 97%, 98% or 99% identity thereof. In one embodiment, an isolated nucleic acid molecule is provided wherein the encoded extracellular mesothelin antigen binding domain comprises an amino acid sequence of SEQ ID NO: 88 or an amino acid sequence with 85%, 90%, 95%, 96%, 97%, 98% or 99% identity to an amino acid sequence of SEQ ID NO: 88.

In another embodiment, the nucleic acid sequence encoding a CAR comprises the nucleic acid sequence of SEQ ID NO: 89 or a sequence with 85%, 90%, 95%, 96%, 97%, 98% or 99% identity thereof, and encodes the CAR comprising the amino acid sequence as set forth in SEQ ID NO: 90 or a sequence with 85%, 90%, 95%, 96%, 97%, 98% or 99% identity thereof. In addition to scFv sequences that may be used as the extracellular antigen binding domain in the CAR-based surface antigen-regulated promoter-therapeutic payload constructs may comprise, single chain antigen binders (as opposed to scFv) can be incorporated can be incorporated into a functional CAR.

For example, the CD33-specific heavy chain only binder, as disclosed in Applicant's co-pending Non-Provisional patent application Ser. No. 15/934,770 (Provisional Patent No. 62/476,438), entitled Compositions and Methods For Treating Cancer With Anti-CD33 Immunotherapy, as filed on Mar. 24, 2018, and assigned Lentigen Technology, Inc. matter number LEN_018.

In one embodiment, the isolated nucleic acid molecule encoding the extracellular CD33 antigen binding domain comprises a nucleotide sequence of SEQ ID NO: 91, or a sequence with 85%, 90%, 95%, 96%, 97%, 98% or 99% identity thereof. In one embodiment, an isolated nucleic acid molecule is provided wherein the encoded extracellular CD33 antigen binding domain comprises an amino acid sequence of SEQ ID NO: 92 or an amino acid sequence with 85%, 90%, 95%, 96%, 97%, 98% or 99% identity to an amino acid sequence of SEQ ID NO: 92.

In another embodiment, the nucleic acid sequence encoding a functional CAR LTG1906 that targets CD33-expressing malignancies comprises the nucleic acid sequence of SEQ ID NO: 93 or a sequence with 85%, 90%, 95%, 96%, 97%, 98% or 99% identity thereof, and encodes the CAR comprising the amino acid sequence as set forth in SEQ ID NO: 94 or a sequence with 85%, 90%, 95%, 96%, 97%, 98% or 99% identity thereof.

In one embodiment, the extracellular antigen binding domain in the CAR-based surface antigen-regulated promoter-therapeutic payload constructs may comprise, for example, the scFV binders as disclosed in Applicant's co-pending Provisional Patent Application No. 62/773,940, entitled Compositions and Methods for Treating Cancer with Anti-CD38 Immunotherapy, as filed on Nov. 30, 2018, and assigned Lentigen Technology, Inc. matter number LEN_026.

In one embodiment, the isolated nucleic acid molecule encoding the extracellular CD38 antigen binding domain M3803 comprises a nucleotide sequence of SEQ ID NO: 144, or a sequence with 85%, 90%, 95%, 96%, 97%, 98% or 99% identity thereof. In one embodiment, an isolated nucleic acid molecule is provided wherein the encoded extracellular CD38 antigen binding domain comprises an amino acid sequence of SEQ ID NO: 145 or an amino acid sequence with 85%, 90%, 95%, 96%, 97%, 98% or 99% identity to an amino acid sequence of SEQ ID NO: 145.

In one embodiment, the isolated nucleic acid molecule encoding the extracellular CD38 antigen binding domain M3804 comprises a nucleotide sequence of SEQ ID NO: 146, or a sequence with 85%, 90%, 95%, 96%, 97%, 98% or 99% identity thereof. In one embodiment, an isolated nucleic acid molecule is provided wherein the encoded extracellular CD38 antigen binding domain comprises an amino acid sequence of SEQ ID NO: 147 or an amino acid sequence with 85%, 90%, 95%, 96%, 97%, 98% or 99% identity to an amino acid sequence of SEQ ID NO: 147.

In one embodiment, the isolated nucleic acid molecule encoding the extracellular CD38 antigen binding domain M3809 comprises a nucleotide sequence of SEQ ID NO: 148, or a sequence with 85%, 90%, 95%, 96%, 97%, 98% or 99% identity thereof. In one embodiment, an isolated nucleic acid molecule is provided wherein the encoded extracellular CD38 antigen binding domain comprises an amino acid sequence of SEQ ID NO: 149 or an amino acid sequence with 85%, 90%, 95%, 96%, 97%, 98% or 99% identity to an amino acid sequence of SEQ ID NO: 149.

In one embodiment, the isolated nucleic acid molecule encoding the extracellular CD38 antigen binding domain M3811 comprises a nucleotide sequence of SEQ ID NO: 150, or a sequence with 85%, 90%, 95%, 96%, 97%, 98% or 99% identity thereof. In one embodiment, an isolated nucleic acid molecule is provided wherein the encoded extracellular CD38 antigen binding domain comprises an amino acid sequence of SEQ ID NO: 151 or an amino acid sequence with 85%, 90%, 95%, 96%, 97%, 98% or 99% identity to an amino acid sequence of SEQ ID NO: 151. In another embodiment, the nucleic acid sequence encoding a functional CAR LTG 2091 that targets CD38-expressing malignancies comprises the nucleic acid sequence of SEQ ID NO: 7 or a sequence with 85%, 90%, 95%, 96%, 97%, 98% or 99% identity thereof, and encodes the CAR comprising the amino acid sequence as set forth in SEQ ID NO: 8 or a sequence with 85%, 90%, 95%, 96%, 97%, 98% or 99% identity thereof.

In another embodiment, the nucleic acid sequence encoding a functional CAR LTG 2092 that targets CD38-expressing malignancies comprises the nucleic acid sequence of SEQ ID NO: 9 or a sequence with 85%, 90%, 95%, 96%, 97%, 98% or 99% identity thereof, and encodes the CAR comprising the amino acid sequence as set forth in SEQ ID NO: 10 or a sequence with 85%, 90%, 95%, 96%, 97%, 98% or 99% identity thereof.

In another embodiment, the nucleic acid sequence encoding a functional CAR LTG 2095 that targets CD38-expressing malignancies comprises the nucleic acid sequence of SEQ ID NO: 11 or a sequence with 85%, 90%, 95%, 96%, 97%, 98% or 99% identity thereof, and encodes the CAR comprising the amino acid sequence as set forth in SEQ ID NO: 12 or a sequence with 85%, 90%, 95%, 96%, 97%, 98% or 99% identity thereof.

In another embodiment, the nucleic acid sequence encoding a functional CAR LTG 2097 that targets CD38-expressing malignancies comprises the nucleic acid sequence of SEQ ID NO: 15 or a sequence with 85%, 90%, 95%, 96%, 97%, 98% or 99% identity thereof, and encodes the CAR comprising the amino acid sequence as set forth in SEQ ID NO: 16 or a sequence with 85%, 90%, 95%, 96%, 97%, 98% or 99% identity thereof.

In one embodiment, the extracellular antigen binding domain in the CAR-based surface antigen-regulated promoter-therapeutic payload constructs may comprise, for example, the scFV binders as disclosed in Applicant's co-pending Non-Provisional patent application Ser. No. 16/179,364, entitled Compositions and Methods for Treating Cancer with Anti-ROR1 Immunotherapy, as filed on Nov. 2, 2018, and assigned Lentigen Technology, Inc. matter number LEN_022.

In one embodiment, the isolated nucleic acid molecule encoding the extracellular ROR1 antigen binding domain comprises a nucleotide sequence of SEQ ID NO: 152, or a sequence with 85%, 90%, 95%, 96%, 97%, 98% or 99% identity thereof. In one embodiment, an isolated nucleic acid molecule is provided wherein the encoded extracellular ROR1 antigen binding domain comprises an amino acid sequence of SEQ ID NO: 153 or an amino acid sequence with 85%, 90%, 95%, 96%, 97%, 98% or 99% identity to an amino acid sequence of SEQ ID NO: 153.

In one embodiment, the isolated nucleic acid molecule encoding the extracellular ROR1 antigen binding domain comprises a nucleotide sequence of SEQ ID NO: 154, or a sequence with 85%, 90%, 95%, 96%, 97%, 98% or 99% identity thereof. In one embodiment, an isolated nucleic acid molecule is provided wherein the encoded extracellular ROR1 antigen binding domain comprises an amino acid sequence of SEQ ID NO: 155 or an amino acid sequence with 85%, 90%, 95%, 96%, 97%, 98% or 99% identity to an amino acid sequence of SEQ ID NO: 155.

In one embodiment, the isolated nucleic acid molecule encoding the extracellular ROR1 antigen binding domain comprises a nucleotide sequence of SEQ ID NO: 156, or a sequence with 85%, 90%, 95%, 96%, 97%, 98% or 99% identity thereof. In one embodiment, an isolated nucleic acid molecule is provided wherein the encoded extracellular ROR1 antigen binding domain comprises an amino acid sequence of SEQ ID NO: 157 or an amino acid sequence with 85%, 90%, 95%, 96%, 97%, 98% or 99% identity to an amino acid sequence of SEQ ID NO: 157.

In another embodiment, the nucleic acid sequence encoding a functional CAR LTG 1941 that targets ROR1-expressing malignancies comprises the nucleic acid sequence of SEQ ID NO: 17 or a sequence with 85%, 90%, 95%, 96%, 97%, 98% or 99% identity thereof, and encodes the CAR comprising the amino acid sequence as set forth in SEQ ID NO: 18 or a sequence with 85%, 90%, 95%, 96%, 97%, 98% or 99% identity thereof.

In another embodiment, the nucleic acid sequence encoding a functional CAR LTG 1942 that targets ROR1-expressing malignancies comprises the nucleic acid sequence of SEQ ID NO: 19 or a sequence with 85%, 90%, 95%, 96%, 97%, 98% or 99% identity thereof, and encodes the CAR comprising the amino acid sequence as set forth in SEQ ID NO: 20 or a sequence with 85%, 90%, 95%, 96%, 97%, 98% or 99% identity thereof.

In another embodiment, the nucleic acid sequence encoding a functional CAR LTG 1943 that targets ROR1-expressing malignancies comprises the nucleic acid sequence of SEQ ID NO: 21 or a sequence with 85%, 90%, 95%, 96%, 97%, 98% or 99% identity thereof, and encodes the CAR comprising the amino acid sequence as set forth in SEQ ID NO: 22 or a sequence with 85%, 90%, 95%, 96%, 97%, 98% or 99% identity thereof.

In one embodiment, the extracellular antigen binding domain in the CAR-based surface antigen-regulated promoter-therapeutic payload constructs may comprise, for example, the scFV binders as disclosed in Applicant's co-pending Provisional Patent Application No. 62/734,106, entitled Compositions and Methods for Treating Cancer with Anti-CD123 Immunotherapy, as filed on Sep. 20, 2018, and assigned Lentigen Technology, Inc. matter number LEN_024.

In one embodiment, the isolated nucleic acid molecule encoding the extracellular CD123 antigen binding domain M12303 comprises a nucleotide sequence of SEQ ID NO: 158, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 159 or an amino acid sequence with 85%, 90%, 95%, 96%, 97%, 98% or 99% identity to an amino acid sequence of SEQ ID NO: 159.

In one embodiment, the isolated nucleic acid molecule encoding the extracellular CD123 antigen binding domain M12304 comprises a nucleotide sequence of SEQ ID NO: 160, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 161 or an amino acid sequence with 85%, 90%, 95%, 96%, 97%, 98% or 99% identity to an amino acid sequence of SEQ ID NO:161.

In one embodiment, the isolated nucleic acid molecule encoding the extracellular CD123 antigen binding domain M12305 comprises a nucleotide sequence of SEQ ID NO: 162, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 163 or an amino acid sequence with 85%, 90%, 95%, 96%, 97%, 98% or 99% identity to an amino acid sequence of SEQ ID NO: 163.

In one embodiment, the isolated nucleic acid molecule encoding the extracellular CD123 antigen binding domain M12306 comprises a nucleotide sequence of SEQ ID NO: 164, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 165 or an amino acid sequence with 85%, 90%, 95%, 96%, 97%, 98% or 99% identity to an amino acid sequence of SEQ ID NO: 165.

In one embodiment, the isolated nucleic acid molecule encoding the extracellular CD123 antigen binding domain M12308 comprises a nucleotide sequence of SEQ ID NO: 166, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 167 or an amino acid sequence with 85%, 90%, 95%, 96%, 97%, 98% or 99% identity to an amino acid sequence of SEQ ID NO: 167.

In one embodiment, the isolated nucleic acid molecule encoding the extracellular CD123 antigen binding domain M12311 comprises a nucleotide sequence of SEQ ID NO: 168, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 169 or an amino acid sequence with 85%, 90%, 95%, 96%, 97%, 98% or 99% identity to an amino acid sequence of SEQ ID NO: 169.

In one embodiment, the isolated nucleic acid molecule encoding the extracellular CD123 antigen binding domain M12313 comprises a nucleotide sequence of SEQ ID NO: 170, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 171 or an amino acid sequence with 85%, 90%, 95%, 96%, 97%, 98% or 99% identity to an amino acid sequence of SEQ ID NO: 171.

In one embodiment, the isolated nucleic acid molecule encoding the extracellular CD123 antigen binding domain M12315 comprises a nucleotide sequence of SEQ ID NO: 172, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 173 or an amino acid sequence with 85%, 90%, 95%, 96%, 97%, 98% or 99% identity to an amino acid sequence of SEQ ID NO: 173.

In one embodiment, the isolated nucleic acid molecule encoding the extracellular CD123 antigen binding domain M12317 comprises a nucleotide sequence of SEQ ID NO: 174, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 175 or an amino acid sequence with 85%, 90%, 95%, 96%, 97%, 98% or 99% identity to an amino acid sequence of SEQ ID NO: 175.

In one embodiment, the isolated nucleic acid molecule encoding the extracellular CD123 antigen binding domain M12318 comprises a nucleotide sequence of SEQ ID NO: 176, or a sequence with 85%, 90%, 95%, 96%, 97%, 98% or 99% identity thereof. In one embodiment, an isolated nucleic acid molecule is provided wherein the encoded extracellular CD123 antigen binding domain comprises an amino acid sequence of SEQ ID NO: 177 or an amino acid sequence with 85%, 90%, 95%, 96%, 97%, 98% or 99% identity to an amino acid sequence of SEQ ID NO: 177.

In another embodiment, the nucleic acid sequence encoding a functional CAR LTG 2075 that targets CD123-expressing malignancies comprises the nucleic acid sequence of SEQ ID NO: 23 or a sequence with 85%, 90%, 95%, 96%, 97%, 98% or 99% identity thereof, and encodes the CAR comprising the amino acid sequence as set forth in SEQ ID NO: 24 or a sequence with 85%, 90%, 95%, 96%, 97%, 98% or 99% identity thereof.

In another embodiment, the nucleic acid sequence encoding a functional CAR LTG 2076 that targets CD123-expressing malignancies comprises the nucleic acid sequence of SEQ ID NO: 25 or a sequence with 85%, 90%, 95%, 96%, 97%, 98% or 99% identity thereof, and encodes the CAR comprising the amino acid sequence as set forth in SEQ ID NO: 26 or a sequence with 85%, 90%, 95%, 96%, 97%, 98% or 99% identity thereof.

In another embodiment, the nucleic acid sequence encoding a functional CAR LTG 2077 that targets CD123-expressing malignancies comprises the nucleic acid sequence of SEQ ID NO: 115 or a sequence with 85%, 90%, 95%, 96%, 97%, 98% or 99% identity thereof, and encodes the CAR comprising the amino acid sequence as set forth in SEQ ID NO: 116 or a sequence with 85%, 90%, 95%, 96%, 97%, 98% or 99% identity thereof.

In another embodiment, the nucleic acid sequence encoding a functional CAR LTG 2078 that targets CD123-expressing malignancies comprises the nucleic acid sequence of SEQ ID NO: 117 or a sequence with 85%, 90%, 95%, 96%, 97%, 98% or 99% identity thereof, and encodes the CAR comprising the amino acid sequence as set forth in SEQ ID NO: 118 or a sequence with 85%, 90%, 95%, 96%, 97%, 98% or 99% identity thereof.

In another embodiment, the nucleic acid sequence encoding a functional CAR LTG 2079 that targets CD123-expressing malignancies comprises the nucleic acid sequence of SEQ ID NO: 119 or a sequence with 85%, 90%, 95%, 96%, 97%, 98% or 99% identity thereof, and encodes the CAR comprising the amino acid sequence as set forth in SEQ ID NO: 120 or a sequence with 85%, 90%, 95%, 96%, 97%, 98% or 99% identity thereof.

In another embodiment, the nucleic acid sequence encoding a functional CAR LTG 2082 that targets CD123-expressing malignancies comprises the nucleic acid sequence of SEQ ID NO: 121 or a sequence with 85%, 90%, 95%, 96%, 97%, 98% or 99% identity thereof, and encodes the CAR comprising the amino acid sequence as set forth in SEQ ID NO: 122 or a sequence with 85%, 90%, 95%, 96%, 97%, 98% or 99% identity thereof.

In another embodiment, the nucleic acid sequence encoding a functional CAR LTG 2083 that targets CD123-expressing malignancies comprises the nucleic acid sequence of SEQ ID NO: 123 or a sequence with 85%, 90%, 95%, 96%, 97%, 98% or 99% identity thereof, and encodes the CAR comprising the amino acid sequence as set forth in SEQ ID NO: 124 or a sequence with 85%, 90%, 95%, 96%, 97%, 98% or 99% identity thereof.

In another embodiment, the nucleic acid sequence encoding a functional CAR LTG 2085 that targets CD123-expressing malignancies comprises the nucleic acid sequence of SEQ ID NO: 125 or a sequence with 85%, 90%, 95%, 96%, 97%, 98% or 99% identity thereof, and encodes the CAR comprising the amino acid sequence as set forth in SEQ ID NO: 126 or a sequence with 85%, 90%, 95%, 96%, 97%, 98% or 99% identity thereof.

In another embodiment, the nucleic acid sequence encoding a functional CAR LTG 2087 that targets CD123-expressing malignancies comprises the nucleic acid sequence of SEQ ID NO: 127 or a sequence with 85%, 90%, 95%, 96%, 97%, 98% or 99% identity thereof, and encodes the CAR comprising the amino acid sequence as set forth in SEQ ID NO: 128 or a sequence with 85%, 90%, 95%, 96%, 97%, 98% or 99% identity thereof.

In another embodiment, the nucleic acid sequence encoding a functional CAR LTG 2088 that targets CD123-expressing malignancies comprises the nucleic acid sequence of SEQ ID NO: 129 or a sequence with 85%, 90%, 95%, 96%, 97%, 98% or 99% identity thereof, and encodes the CAR comprising the amino acid sequence as set forth in SEQ ID NO: 130 or a sequence with 85%, 90%, 95%, 96%, 97%, 98% or 99% identity thereof.

In one embodiment, the extracellular antigen binding domain in the CAR-based surface antigen-regulated promoter-therapeutic payload constructs may comprise, for example, the scFV binders as disclosed in Applicant's co-pending Provisional Patent Application No. 62/736,955, entitled Compositions and Methods for Treating Cancer with Human Anti-CD19/22 Immunotherapy, as filed on Sep. 26, 2018, and assigned Lentigen Technology, Inc. matter number LEN_025.

In one embodiment, the isolated nucleic acid molecule encoding the extracellular CD19/CD22 antigen binding domain comprises a nucleotide sequence SEQ ID NO: 178, or a sequence with 85%, 90%, 95%, 96%, 97%, 98% or 99% identity thereof. In one embodiment, an isolated nucleic acid molecule is provided wherein the encoded extracellular mesothelin antigen binding domain comprises an amino acid sequence of SEQ ID NO: 179 or an amino acid sequence with 85%, 90%, 95%, 96%, 97%, 98% or 99% identity to an amino acid sequence of SEQ ID: 179.

In another embodiment, the nucleic acid sequence encoding a functional CAR LTG 2737 that targets CD19/CD22-expressing malignancies comprises the nucleic acid sequence of SEQ ID NO: 131 or a sequence with 85%, 90%, 95%, 96%, 97%, 98% or 99% identity thereof, and encodes the CAR comprising the amino acid sequence as set forth in SEQ ID NO: 132 or a sequence with 85%, 90%, 95%, 96%, 97%, 98% or 99% identity thereof.

In one embodiment, the extracellular antigen binding domain in the CAR-based surface antigen-regulated promoter-therapeutic payload constructs may comprise, for example, the scFV binders as disclosed in Applicant's co-pending Non-Provisional patent application Ser. No. 16/161,542, entitled Compositions and Methods for Treating Cancer with Human Anti-CD22 Immunotherapy, as filed on Oct. 16, 2018, and assigned Lentigen Technology, Inc. matter number LEN_021.

In one embodiment, the isolated nucleic acid molecule encoding the extracellular CD22 antigen binding domain 16P17 comprises a nucleotide sequence SEQ ID NO: 180, or a sequence with 85%, 90%, 95%, 96%, 97%, 98% or 99% identity thereof. In one embodiment, an isolated nucleic acid molecule is provided wherein the encoded extracellular mesothelin antigen binding domain comprises an amino acid sequence of SEQ ID NO: 181 or an amino acid sequence with 85%, 90%, 95%, 96%, 97%, 98% or 99% identity to an amino acid sequence of SEQ ID: 181.

In one embodiment, the isolated nucleic acid molecule encoding the extracellular CD22 antigen binding domain 16P13 comprises a nucleotide sequence SEQ ID NO: 182, or a sequence with 85%, 90%, 95%, 96%, 97%, 98% or 99% identity thereof. In one embodiment, an isolated nucleic acid molecule is provided wherein the encoded extracellular mesothelin antigen binding domain comprises an amino acid sequence of SEQ ID NO: 183 or an amino acid sequence with 85%, 90%, 95%, 96%, 97%, 98% or 99% identity to an amino acid sequence of SEQ ID: 183.

In another embodiment, the nucleic acid sequence encoding a functional CAR LTG 2209 that targets CD22-expressing malignancies comprises the nucleic acid sequence of SEQ ID NO: 133 or a sequence with 85%, 90%, 95%, 96%, 97%, 98% or 99% identity thereof, and encodes the CAR comprising the amino acid sequence as set forth in SEQ ID NO: 134 or a sequence with 85%, 90%, 95%, 96%, 97%, 98% or 99% identity thereof.

In another embodiment, the nucleic acid sequence encoding a functional CAR LTG 2219 that targets CD22-expressing malignancies comprises the nucleic acid sequence of SEQ ID NO: 135 or a sequence with 85%, 90%, 95%, 96%, 97%, 98% or 99% identity thereof, and encodes the CAR comprising the amino acid sequence as set forth in SEQ ID NO: 136 or a sequence with 85%, 90%, 95%, 96%, 97%, 98% or 99% identity thereof.

In one embodiment, the extracellular antigen binding domain in the CAR-based surface antigen-regulated promoter-therapeutic payload constructs may comprise, for example, the scFV binders as disclosed in Applicant's co-pending Non-Provisional patent application Ser. No. 16/050,754, entitled Compositions and Methods for Treating Cancer with Anti-CD19/20 Immunotherapy, as filed on Jul. 31, 2018, and assigned Lentigen Technology, Inc. matter number LEN_019.

In one embodiment, the isolated nucleic acid molecule encoding the extracellular CD19/CD20 antigen binding domain comprises a nucleotide sequence SEQ ID NO: 141, or a sequence with 85%, 90%, 95%, 96%, 97%, 98% or 99% identity thereof. In one embodiment, an isolated nucleic acid molecule is provided wherein the encoded extracellular mesothelin antigen binding domain comprises an amino acid sequence of SEQ ID NO: 112 or an amino acid sequence with 85%, 90%, 95%, 96%, 97%, 98% or 99% identity to an amino acid sequence of SEQ ID: 112.

In one embodiment, the isolated nucleic acid molecule encoding the extracellular CD19/CD20 antigen binding domain comprises a nucleotide sequence SEQ ID NO: 113, or a sequence with 85%, 90%, 95%, 96%, 97%, 98% or 99% identity thereof. In one embodiment, an isolated nucleic acid molecule is provided wherein the encoded extracellular mesothelin antigen binding domain comprises an amino acid sequence of SEQ ID NO: 114 or an amino acid sequence with 85%, 90%, 95%, 96%, 97%, 98% or 99% identity to an amino acid sequence of SEQ ID: 114.

In another embodiment, the nucleic acid sequence encoding a functional CAR LTG 1496 that targets CD19/CD20-expressing malignancies comprises the nucleic acid sequence of SEQ ID NO: 95 or a sequence with 85%, 90%, 95%, 96%, 97%, 98% or 99% identity thereof, and encodes the CAR comprising the amino acid sequence as set forth in SEQ ID NO: 96 or a sequence with 85%, 90%, 95%, 96%, 97%, 98% or 99% identity thereof.

In another embodiment, the nucleic acid sequence encoding a functional CAR LTG 1497 that targets CD19/CD20-expressing malignancies comprises the nucleic acid sequence of SEQ ID NO: 97 or a sequence with 85%, 90%, 95%, 96%, 97%, 98% or 99% identity thereof, and encodes the CAR comprising the amino acid sequence as set forth in SEQ ID NO: 98 or a sequence with 85%, 90%, 95%, 96%, 97%, 98% or 99% identity thereof.

In yet another embodiment, the nucleic acid sequence encoding a CAR-based surface antigen-regulated promoter-therapeutic payload construct comprises one or more of the nucleic acid sequences disclosed in Applicant's co-pending Continuation-In-Part Patent application Ser. No. 16/134,735, filed Sep. 18, 2018, entitled Compositions and Methods for Treating Cancer with DuoCARs, which claims priority to PCT Application No. PCT/US17/49923, filed Sep. 1, 2017, which in turn claims the benefit of priority under 35 U.S.C. Section 119(e) to U.S. Provisional Patent Application No. 62/382,791 filed on Sep. 2, 2016, the entire contents of each of which are incorporated herein by reference.

Thus, in one embodiment, variant single specificity-based CAR structures that are known to be compatible in Applicant's DuoCAR setting may also utilized to generate the CAR-based surface antigen-regulated promoter-therapeutic payload constructs. Specific examples of the single specificity therapeutic surface antigen-regulated promoter-payload constructs (e.g. CAR-based) on which a DuoCAR-based surface antigen-regulated promoter-therapeutic payload construct technology may be based include the single CD20 targeting vector LTG1495, nucleotide sequence SEQ ID NO: 142 and amino acid sequence SEQ ID NO: 143. A second example is the single specificity CAR LTG2200, specific for CD22, nucleotide sequence SEQ ID NO: 69 and amino acid sequence SEQ ID NO: 70.

In yet another embodiment, variant CAR structures that are known to be compatible in the DuoCAR setting a may also utilized to generate the CAR-based surface antigen-regulated promoter-therapeutic payload constructs included within the scope of this disclosure. These include the CD19-specific CAR LTG1494 described in nucleotide sequence SEQ ID NO: 71 and amino acid sequence SEQ ID NO: 72, respectively. This sequence includes the well-described linker that joins the heavy and light chains of the scFv referred to as the Whitlow linker (amino acid sequence GSTSGSGKPGSGEGSTKG (SEQ ID NO: 184), see Whitlow M., et al., 1993, Protein Eng. 6:989-995). In some cases the Whitlow linker was substituted for a (GGGGS)n linker (SEQ ID NO: 185), for example in a CD19 CAR format, as in LTG1538, nucleotide sequence SEQ ID NO: 73 and amino acid sequence SEQ ID NO: 74, respectively. In another example CARs were created that have alternate transmembrane domains. The anti-CD19 CAR LTG1562, nucleotide sequence SEQ ID NO: 75 and amino acid sequence SEQ ID NO: 76, respectively, utilizes the CD4 (as opposed to CD8) transmembrane domain. Similarly the anti-CD19 CAR LTG1563 has an alternate transmembrane derived from TNFRSF19, nucleotide sequence SEQ ID NO: 77 and amino acid sequence SEQ ID NO: 78, respectively.

In yet another embodiment, another example of a therapeutic application would be the treatment of leukemia that expresses the CD19, CD20, and TSLPR antigens with DuoCAR-based surface antigen-regulated promoter-therapeutic payload constructs of the present invention. In particular, a DuoCAR-based surface antigen-regulated promoter-therapeutic payload construct(s) comprises LTG1496 or LTG 1497 (SEQ ID NOs: 95, 97, respectively) combined with a TSLPR-specific CAR (LTG1789), SEQ ID NO: 101 and amino acid sequence SEQ ID NO: 102, respectively, that has been created from TSLPR-specific scFV domains, nucleotide sequence SEQ ID NO: 99 and amino acid sequence SEQ ID NO: 100.

In one embodiment, each of the aforementioned DuoCAR-based surface antigen-regulated promoter-therapeutic payload constructs, the respective CAR constructs are self-driven by a single surface antigen-regulated promoter with separation of the CAR constructs by a ribosome 2A skip site therebetween.

In another embodiment, each of the aforementioned DuoCAR-based surface antigen-regulated promoter-therapeutic payload constructs, the respective CAR constructs are self-driven by separate surface antigen-regulated promoters.

In certain embodiments, as used herein, a non-limiting example of an anti-cd19 CAR construct includes the anti-cd19 CAR construct encoded by the nucleotide sequence referred to herein as LTG1563 (c.f, SEQ ID NO. 77) and which encodes the anti-cd19 CAR construct identified herein as CAR-LTG1563 (c.f., SEQ ID NO. 78).

In one embodiment, the construction of a surface antigen-regulated inducible promoter-therapeutic payload construct comprising the nucleic acid sequence of SEQ ID NO: 139 and encoding the CAR-based promoter-therapeutic payload construct comprising the amino acid sequence of SEQ ID NO: 78 (CAR LTG1563) is described in Example 1 infra.

In one embodiment, the construction of a surface antigen-regulated inducible promoter-therapeutic payload construct comprising the nucleic acid sequence of SEQ ID NO: 140 and encoding the CAR-based promoter-therapeutic payload construct comprising the amino acid sequence of SEQ ID NO: 78 (CAR LTG1563) is described in Example 1 infra.

In one embodiment, the construction of a surface antigen-regulated inducible promoter-therapeutic payload construct comprising the nucleic acid sequence of SEQ ID NO: 77 and encoding the CAR-based promoter-therapeutic payload construct comprising the amino acid sequence of SEQ ID NO: 78 (CAR LTG1563) is described in Example 1 infra.

Without being intended to limit to any particular mechanism of action, it is believed that possible reasons for the enhanced therapeutic function associated with the exemplary surface antigen-regulated promoter-therapeutic payload constructs of the invention include, for example, and not by way of limitation, a) improved lateral movement within the plasma membrane allowing for more efficient signal transduction, b) superior location within plasma membrane microdomains, such as lipid rafts, and greater ability to interact with transmembrane signaling cascades associated with T cell activation, c) superior location within the plasma membrane by preferential movement away from dampening or down-modulatory interactions, such as less proximity to or interaction with phosphatases such as CD45, and d) superior assembly into T cell receptor signaling complexes (e.g. the immune synapse), or any combination thereof.

Depending on the desired antigen to be targeted, the surface antigen-regulated promoter-therapeutic payload construct can be engineered to include the appropriate antigen binding domain that is specific to the desired antigen target. For example, and not by way of limitation, if CD19 is the desired antigen that is to be targeted, an antibody for CD19 can be used as the antigen binding domain incorporated into the surface antigen-regulated promoter-therapeutic payload construct.

In one exemplary embodiment, the antigen binding domain portion of the CAR-based surface antigen-regulated promoter-therapeutic payload construct targets CD19. Preferably, the extracellular antigen binding domain in the surface antigen-regulated promoter-therapeutic payload construct is anti-CD19 scFV, wherein the nucleic acid sequence of the extracellular anti-CD19 scFV comprises the sequence set forth in SEQ ID NO: 37 or a sequence with 85%, 90%, 95%, 96%, 97%, 98% or 99% identity thereof. In one embodiment, the extracellular anti-CD19 scFV comprises the nucleic acid sequence that encodes the amino acid sequence of SEQ ID NO: 38 or a sequence with 85%, 90%, 95%, 96%, 97%, 98% or 99% identity thereof. In another embodiment, the extracellular anti-CD19 scFV portion of the CAR comprises the amino acid sequence set forth in SEQ ID NO: 38 or a sequence with 85%, 90%, 95%, 96%, 97%, 98% or 99% identity thereof.

In one aspect of the present invention, there is provided a surface antigen-regulated promoter-therapeutic payload construct capable of binding to a non-TSA or non-TAA including, for example and not by way of limitation, an antigen derived from Retroviridae (e.g. human immunodeficiency viruses such as HIV-1 and HIV-LP), Picornaviridae (e.g. poliovirus, hepatitis A virus, enterovirus, human coxsackievirus, rhinovirus, and echovirus), rubella virus, coronavirus, vesicular stomatitis virus, rabies virus, ebola virus, parainfluenza virus, mumps virus, measles virus, respiratory syncytial virus, influenza virus, hepatitis B virus, parvovirus, Adenoviridae, Herpesviridae (e.g. type 1 and type 2 herpes simplex virus (HSV), varicella-zoster virus, cytomegalovirus (CMV), and herpes virus), Poxviridae (e.g. smallpox virus, vaccinia virus, and pox virus), or hepatitis C virus, or any combination thereof.

In another aspect of the present invention, there is provided a surface antigen-regulated promoter-therapeutic payload construct capable of binding to an antigen derived from a bacterial strain of Staphylococci, Streptococcus, Escherichia coli, Pseudomonas, or Salmonella. Particularly, there is provided a surface antigen-regulated promoter-therapeutic payload construct capable of binding to an antigen derived from an infectious bacterium, for example, Helicobacter pyloris, Legionella pneumophilia, a bacterial strain of Mycobacteria sps. (e.g. M. tuberculosis, M. avium, M. intracellulare, M. kansaii, or M. gordonea), Staphylococcus aureus, Neisseria gonorrhoeae, Neisseria meningitides, Listeria monocytogenes, Streptococcus pyogenes, Group A Streptococcus, Group B Streptococcus (Streptococcus agalactiae), Streptococcus pneumoniae, or Clostridium tetani, or a combination thereof.

### 2. Transmembrane Domain

With respect to the transmembrane domain, the CAR-based surface antigen-regulated promoter-therapeutic payload construct comprises one or more TNFRSF transmembrane domains fused to the extracellular domain of the surface antigen-regulated promoter-therapeutic payload construct.

In one embodiment, the TNFRSF transmembrane domain comprises at least one TNFRSF19 transmembrane domain. In one embodiment, an isolated nucleic acid molecule is provided wherein the encoded TNFRSF transmembrane domain comprises a TNFRSF19 transmembrane domain.

In one embodiment, the isolated nucleic acid molecule encoding the TNFRSF19 transmembrane domain comprises a nucleotide sequence of SEQ ID NO: 51, or a sequence with 85%, 90%, 95%, 96%, 97%, 98% or 99% identity thereof. In one embodiment, an isolated nucleic acid molecule is provided wherein the encoded TNFRSF19 transmembrane domain comprises an amino acid sequence of SEQ ID NO: 52, or an amino acid sequence with 85%, 90%, 95%, 96%, 97%, 98% or 99% identity to an amino acid sequence of SEQ ID NO: 52.

The transmembrane domain may be derived either from a natural or from a synthetic source. Where the source is natural, the domain may be derived from any membrane-bound or transmembrane protein.

Transmembrane regions of particular use in the surface antigen-regulated promoter-therapeutic payload construct described herein may be derived from (i.e. comprise) at least the transmembrane region(s) of) the alpha, beta or zeta chain of the T-cell receptor, CD28, CD3 epsilon, CD45, CD4, CD5, CD8, CD9, CD16, CD22, mesothelin, CD33, CD37, CD64, CD80, CD86, CD134, CD137, CD154. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the surface antigen-regulated promoter-therapeutic payload construct. A glycine-serine doublet provides a particularly suitable linker.

In one embodiment, the transmembrane domain that naturally is associated with one of the domains in the surface antigen-regulated promoter-therapeutic payload construct is used in addition to the transmembrane domains described supra.

In some instances, the transmembrane domain can be selected by amino acid substitution to avoid binding of such domains to the transmembrane domains of the same or different surface membrane proteins to minimize interactions with other members of the receptor complex.

In one embodiment, the transmembrane domain in the surface antigen-regulated promoter-therapeutic payload construct of the invention is the CD8 transmembrane domain. In one embodiment, the CD8 transmembrane domain comprises the nucleic acid sequence of SEQ ID NO: 27. In one embodiment, the CD8 transmembrane domain comprises the nucleic acid sequence that encodes the amino acid sequence of SEQ ID NO: 28. In another embodiment, the CD8 transmembrane domain comprises the amino acid sequence of SEQ ID NO: 28.

In one embodiment, the encoded transmembrane domain comprises an amino acid sequence having at least one, two or three modifications (e.g., substitutions) but not more than 20, 10 or 5 modifications (e.g., substitutions) of an amino acid sequence of SEQ ID NO: 28, or a sequence with 95-99% identity to an amino acid sequence of SEQ ID NO: 28.

In some instances, the transmembrane domain of the CAR comprises the CD8.alpha.hinge domain. In one embodiment, the CD8 hinge domain comprises the nucleic acid sequence of SEQ ID NO: 29. In one embodiment, the CD8 hinge domain comprises the nucleic acid sequence that encodes the amino acid sequence of SEQ ID NO: 30. In another embodiment, the CD8 hinge domain comprises the amino acid sequence of SEQ ID NO: 30, or a sequence with 95-99% identify thereof.

In one embodiment, an isolated nucleic acid molecule is provided wherein the encoded linker domain is derived from the extracellular domain of CD8, and is linked to the transmembrane CD8 domain, the transmembrane CD28 domain, or a combination thereof.

In one embodiment of the patient-specific autologous anti-tumor lymphocyte cell population(s) as disclosed herein, non-limiting exemplary transmembrane domains for use in the CAR-based surface antigen-regulated promoter-therapeutic payload constructs disclosed herein include the TNFRSF16 and TNFRSF19 transmembrane domains may be used to derive the TNFRSF transmembrane domains and/or linker or spacer domains as disclosed in Applicant's co-pending patent application Ser. No. 15/767,076, entitled CHIMERIC ANTIGEN RECEPTORS AND METHODS OF USE, as filed on Apr. 9, 2018, and assigned Lentigen Technology, Inc. matter number LEN_015 (US), including, in particular, those other TNFRSF members listed within the tumor necrosis factor receptor superfamily as listed in Table I therein.

In one embodiment, the transmembrane domain in the CAR of the invention is the TNFRSF19 transmembrane domain. In one embodiment, the TNFRSF19 transmembrane domain comprises the nucleic acid sequence of SEQ ID NO: 51. In one embodiment, the TNFRSF19 transmembrane domain comprises the nucleic acid sequence that encodes the amino acid sequence of SEQ ID NO: 52. In another embodiment, the TNFRSF19 transmembrane domain comprises the amino acid sequence of SEQ ID NO: 52.

In one embodiment, the encoded transmembrane domain comprises an amino acid sequence having at least one, two or three modifications (e.g., substitutions) but not more than 20, 10 or 5 modifications (e.g., substitutions) of an amino acid sequence of SEQ ID NO: 52, or a sequence with 95-99% identity to an amino acid sequence of SEQ ID NO: 52.

### 3. Spacer Domain

In the CAR-based surface antigen-regulated promoter-therapeutic payload construct, a spacer domain can be arranged between the extracellular domain and the TNFRSF transmembrane domain, or between the intracellular domain and the TNFRSF transmembrane domain. The spacer domain means any oligopeptide or polypeptide that serves to link the TNFRSF transmembrane domain with the extracellular domain and/or the TNFRSF transmembrane domain with the intracellular domain. The spacer domain comprises up to 300 amino acids, preferably 10 to 100 amino acids, and most preferably 25 to 50 amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,5667, 498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

The spacer domain preferably has a sequence that promotes binding of a CAR-based surface antigen-regulated promoter-therapeutic payload construct with an antigen and enhances signaling into a cell. Examples of an amino acid that is expected to promote the binding include cysteine, a charged amino acid, and serine and threonine in a potential glycosylation site, and these amino acids can be used as an amino acid constituting the spacer domain.

As the spacer domain, the entire or a part of amino acid numbers 118 to 178 (SEQ ID NO: 31) which is a hinge region of CD8.alpha. (NCBI RefSeq: NP.sub.--001759.3), amino acid numbers 135 to 195 of CD8.beta. (GenBank: AAA35664.1), amino acid numbers 315 to 396 of CD4 (NCBI RefSeq: NP.sub.--000607.1), or amino acid numbers 137 to 152 of CD28 (NCBI RefSeq: NP.sub.--006130.1) can be used. Also, as the spacer domain, a part of a constant region of an antibody H chain or L chain (CH1 region or CL region, for example, a peptide having an amino acid sequence shown in SEQ ID NO: 32) can be used. Further, the spacer domain may be an artificially synthesized sequence.

In addition, an entire or a part of amino acids comprising the constant region of a human IgG4 (UniProt ID: P01861), including CH1, (amino acid numbers 1-98), hinge, SEQ ID NO: 80, and the corresponding nucleotide SEQ ID NO:79, (amino acid numbers 99-110), CH2, amino acid SEQ ID NO: 81 and corresponding nucleotide SEQ ID NO: 80, (amino acid numbers 111-220) and CH3, SEQ ID NO:84 and corresponding nucleotide SEQ ID NO: 83, (amino acid numbers 221-327) or a combination thereof, such as IgG4 Hinge CH2 CH3 domain, SEQ ID NO: 86, and the corresponding nucleotide SEQ ID NO: 85, can be used.

In one embodiment, the spacer domain of the CAR comprises the TNFRSF19 hinge domain which comprises the nucleic acid sequence of SEQ ID NO: 53. In one embodiment, the TNFRSF19 hinge domain comprises the nucleic acid sequence that encodes the amino acid sequence of SEQ ID NO: 54. In another embodiment, the TNFRSF19 hinge domain comprises the amino acid sequence of SEQ ID NO: 54, or a sequence with 95-99% identify thereof.

In one embodiment, the spacer domain of the CAR comprises the TNFRSF19 truncated hinge domain comprises the nucleic acid sequence of SEQ ID NO: 55. In one embodiment, the TNFRSF19 truncated hinge domain comprises the nucleic acid sequence that encodes the amino acid sequence of SEQ ID NO: 56. In another embodiment, the TNFRSF19 truncated hinge domain comprises the amino acid sequence of SEQ ID NO: 56, or a sequence with 95-99% identify thereof.

In one embodiment, the TNFRSF19 hinge and transmembrane domains comprise the nucleic acid sequence of SEQ ID NO: 49. In one embodiment, the TNFRSF19 hinge and transmembrane domains comprise the nucleic acid sequence that encodes the amino acid sequence of SEQ ID NO: 50. In another embodiment, the TNFRSF19 hinge and transmembrane domains comprise the amino acid sequence of SEQ ID NO: 50, or a sequence with 95-99% identify thereof.

In one embodiment, a CD8a hinge domain is fused to a TNFRSF19 transmembrane domain comprising the nucleic acid sequence of SEQ ID NO: 57. In one embodiment, the CD8a hinge domain is fused to a TNFRSF19 transmembrane domain comprises the nucleic acid sequence that encodes the amino acid sequence of SEQ ID NO: 58. In another embodiment, the CD8a hinge domain is fused to a TNFRSF19 transmembrane domain comprises the amino acid sequence of SEQ ID NO: 58, or a sequence with 95-99% identify thereof.

Further, in the surface antigen-regulated promoter-therapeutic payload construct, a signal peptide sequence, also termed leader peptide, can be linked to the N-terminus. The signal peptide sequence exists at the N-terminus of many secretory proteins and membrane proteins, and has a length of 15 to 30 amino acids. Since many of the protein molecules mentioned above as the intracellular domain have signal peptide sequences, the signal peptides can be used as a signal peptide for the surface antigen-regulated promoter-therapeutic payload construct. In one embodiment, the signal peptide comprises the amino acid sequence shown in SEQ ID NO: 14).

In one embodiment, the CD8 alpha leader peptide, is comprising the nucleic acid sequence of SEQ ID NO: 43. In one embodiment, CD8 alpha leader peptide comprises the nucleic acid sequence that encodes the amino acid sequence of SEQ ID NO: 44. In another embodiment, the CD8a hinge domain is fused to a TNFRSF19 transmembrane domain comprises the amino acid sequence of SEQ ID NO: 44, or a sequence with 95-99% identify thereof.

In another embodiment, the GMCSF leader peptide, is comprising the nucleic acid sequence of SEQ ID NO: 39. In one embodiment, the GMCSF leader peptide, comprises the nucleic acid sequence that encodes the amino acid sequence of SEQ ID NO: 40. In another embodiment, the CD8a hinge domain is fused to a TNFRSF19 transmembrane domain comprises the amino acid sequence of SEQ ID NO: 40, or a sequence with 95-99% identify thereof.

In another embodiment, the TNFRSF19 leader peptide is comprising the nucleic acid sequence of SEQ ID NO: 41. In one embodiment, TNFRSF19 leader peptide, and CD8 alpha leader peptide comprises the nucleic acid sequence that encodes the amino acid sequence of SEQ ID NO: 42. In another embodiment, the CD8a hinge domain is fused to a TNFRSF19 transmembrane domain comprises the amino acid sequence of SEQ ID NO: 42, or a sequence with 95-99% identify thereof.

In one embodiment, a tag sequence encoding a truncated sequence of epidermal growth factor receptor (tEGFR) is comprising the nucleic acid sequence of SEQ ID NO: 67. In one embodiment, tEGFR comprises the nucleic acid sequence that encodes the amino acid sequence of SEQ ID NO: 68. In another embodiment, the tEGFR tag comprises the amino acid sequence of SEQ ID NO: 68, or a sequence with 95-99% identify thereof.

In one embodiment, a furin recognition site and downstream T2A self-cleaving peptide sequence, designed for simultaneous bicistronic expression of the tag sequence and the therapeutic payload sequence, is comprising the nucleic acid sequence of SEQ ID NO: 65. In one embodiment, furin and T2A sequence comprises the nucleic acid sequence that encodes the amino acid sequence of SEQ ID NO: 66. In another embodiment, the tEGFR tag comprises the amino acid sequence of SEQ ID NO: 66 or a sequence with 95-99% identify thereof.

In one embodiment, an upstream furin recognition site and T2A self-cleaving peptide sequence and a furin recognition downstream site, designed for simultaneous bicistronic expression of the tag sequence and the CAR sequence, is comprising the nucleic acid sequence of SEQ ID NO: 67. In one embodiment, furin and T2A sequence comprises the nucleic acid sequence that encodes the amino acid sequence of SEQ ID NO: 68. In another embodiment, the tEGFR tag comprises the amino acid sequence of SEQ ID NO: 68 or a sequence with 95-99% identify thereof.

In one embodiment, the targeting domain of the CAR-based surface antigen-regulated promoter-therapeutic payload construct is expressed separately in the form of monoclonal antibody, ScFv Fab, Fab′2 and is containing at binding tag or epitope, whereas the effector-cell expressed component of the surface antigen-regulated promoter-therapeutic payload construct contains a binding domain specifically directed to bind the tag or epitope expressed on the soluble CAR module, such as specific binding on the soluble component of the CAR to the cell bound component forms the full functional CAR structure.

### 4. Intracellular Domain

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the CARs disclosed herein include those derived from TCR zeta (CD3 Zeta), FcR gamma, FcR beta, CD3 gamma, CD3 delta, CD3 epsilon, CD5, CD22, CD79a, CD79b, and CD66d. Specific, non-limiting examples, of the ITAM include peptides having sequences of amino acid numbers 51 to 164 of CD3.zeta. (NCBI RefSeq: NP.sub.--932170.1), amino acid numbers 45 to 86 of Fc.epsilon.RI.gamma. (NCBI RefSeq: NP.sub.--004097.1), amino acid numbers 201 to 244 of Fc.epsilon.RI.beta. (NCBI RefSeq: NP.sub.--000130.1), amino acid numbers 139 to 182 of CD3.gamma. (NCBI RefSeq: NP.sub.--000064.1), amino acid numbers 128 to 171 of CD3.delta. (NCBI RefSeq: NP.sub.--000723.1), amino acid numbers 153 to 207 of CD3.epsilon. (NCBI RefSeq: NP.sub.--000724.1), amino acid numbers 402 to 495 of CD5 (NCBI RefSeq: NP.sub.--055022.2), amino acid numbers 707 to 847 of 0022 (NCBI RefSeq: NP.sub.--001762.2), amino acid numbers 166 to 226 of CD79a (NCBI RefSeq: NP.sub.--001774.1), amino acid numbers 182 to 229 of CD79b (NCBI RefSeq: NP.sub.--000617.1), and amino acid numbers 177 to 252 of CD66d (NCBI RefSeq: NP.sub.--001806.2), and their variants having the same function as these peptides have. The amino acid number based on amino acid sequence information of NCBI RefSeq ID or GenBank described herein is numbered based on the full length of the precursor (comprising a signal peptide sequence etc.) of each protein. In one embodiment, the cytoplasmic signaling molecule in the CAR comprises a cytoplasmic signaling sequence derived from CD3 zeta.

In a preferred embodiment, the intracellular domain of the CAR can be designed to comprise the CD3-zeta signaling domain by itself or combined with any other desired cytoplasmic domain(s) useful in the context of the CAR. For example, the intracellular domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such costimulatory molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Specific, non-limiting examples, of such costimulatory molecules include peptides having sequences of amino acid numbers 236 to 351 of CD2 (NCBI RefSeq: NP.sub.--001758.2), amino acid numbers 421 to 458 of CD4 (NCBI RefSeq: NP.sub.--000607.1), amino acid numbers 402 to 495 of CD5 (NCBI RefSeq: NP.sub.--055022.2), amino acid numbers 207 to 235 of CD8.alpha. (NCBI RefSeq: NP.sub.--001759.3), amino acid numbers 196 to 210 of CD83 (GenBank: AAA35664.1), amino acid numbers 181 to 220 of CD28 (NCBI RefSeq: NP.sub.--006130.1), amino acid numbers 214 to 255 of CD137 (4-1BB, NCBI RefSeq: NP.sub.--001552.2), amino acid numbers 241 to 277 of CD134 (OX40, NCBI RefSeq: NP.sub.--003318.1), and amino acid numbers 166 to 199 of ICOS (NCBI RefSeq: NP.sub.--036224.1), and their variants having the same function as these peptides have. Thus, while the disclosure herein is exemplified primarily with 4-1BB as the co-stimulatory signaling element, other costimulatory elements are within the scope of the disclosure.

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the intracellular domain is designed to comprise the signaling domain of CD3-zeta and the signaling domain of CD28. In another embodiment, the intracellular domain is designed to comprise the signaling domain of CD3-zeta and the signaling domain of 4-1BB. In yet another embodiment, the intracellular domain is designed to comprise the signaling domain of CD3-zeta and the signaling domain of CD28 and 4-1BB.

In one embodiment, the intracellular domain in the CAR is designed to comprise the signaling domain of 4-1BB and the signaling domain of CD3-zeta, wherein the signaling domain of 4-1BB comprises the nucleic acid sequence set forth in SEQ ID NO: 33, SEQ ID NO: 45, or SEQ ID NO: 59, respectively and the signaling domain of CD3-zeta comprises the nucleic acid sequence set forth in SEQ ID NO: 35, SEQ ID NO: 47, or SEQ ID NO: 61, respectively.

In one embodiment, the intracellular domain in the CAR is designed to comprise the signaling domain of 4-1BB and the signaling domain of CD3-zeta, wherein the signaling domain of 4-1BB comprises the nucleic acid sequence that encodes the amino acid sequence of SEQ ID NO: 34, SEQ ID NO: 46, or SEQ ID NO: 60, respectively and the signaling domain of CD3-zeta comprises the nucleic acid sequence that encodes the amino acid sequence of SEQ ID NO: 36, or SEQ ID NO: 48, or SEQ ID NO: 62.

In one embodiment, the intracellular domain in the CAR is designed to comprise the signaling domain of 4-1BB and the signaling domain of CD3-zeta, wherein the signaling domain of 4-1BB comprises the amino acid sequence set forth in SEQ ID NO: 34, SEQ ID NO: 46, or SEQ ID NO: 60, respectively and the signaling domain of CD3-zeta comprises the amino acid sequence set forth in SEQ ID NO: 36, SEQ ID NO: 48, or SEQ ID NO: 62, respectively.

In one embodiment, the intracellular domain in the CAR is designed to comprise the signaling domain of CD28 and the signaling domain of CD3-zeta, wherein the signaling domain of CD28 comprises the nucleic acid sequence set forth in SEQ ID NO: 45, or SEQ ID NO: 59, respectively, and the signaling domain of CD3-zeta comprises the nucleic acid sequence set forth in SEQ ID NO: 35, SEQ ID NO: 47, or SEQ ID NO: 61, respectively.

In one embodiment, the intracellular domain in the CAR is designed to comprise the signaling domain of CD28 and the signaling domain of CD3-zeta, wherein the signaling domain of CD28 comprises the nucleic acid sequence that encodes the amino acid sequence of SEQ ID NO: 46, or SEQ ID NO: 60, respectively and the signaling domain of CD3-zeta comprises the nucleic acid sequence that encodes the amino acid sequence of SEQ ID NO: 36, or SEQ ID NO: 48, or SEQ ID NO: 62.

In one embodiment, the intracellular domain in the CAR is designed to comprise the signaling domain of CD28 and the signaling domain of CD3-zeta, wherein the signaling domain of CD28 comprises the amino acid sequence set forth in SEQ ID NO: 46, or SEQ ID NO: 60, respectively and the signaling domain of CD3-zeta comprises the amino acid sequence set forth in SEQ ID NO: 36, SEQ ID NO: 48, or SEQ ID NO: 62, respectively.

5. CARs with Additional Auxiliary Components

In another embodiment, the construction of a surface antigen-regulated inducible promoter-therapeutic payload construct comprising additional auxiliary components comprising dominant negative receptors lacking the intracellular signaling domains of either TGFBRII (TGFBRIIdn) and/or PD1 (PD1dn) which were co-expressed with the CAR LTG1563 construct, via ribosomal skipping sites (P2A) to generate a surface antigen-regulated inducible promoter-therapeutic payload construct comprising the nucleic acid sequence of SEQ ID NO: 103, 105, 107, or a combination thereof, and encoding the CAR-based promoter-therapeutic payload construct comprising the amino acid sequence of SEQ ID NO: 104, 106, 108, or a combination thereof, is described in Example 2 infra.

In another embodiment, the surface antigen-regulated inducible promoter-therapeutic payload construct comprising additional auxiliary components comprising dominant negative receptors lacking the intracellular signaling domains of either TGFBRII (TGFBRIIdn) or PD1 (PD1dn) which are co-expressed with the CAR LTG1563 construct, via skipping sites (P2A) to generate a surface antigen-regulated inducible promoter-therapeutic payload construct comprising the nucleic acid sequence of SEQ ID NO: 103, 105, 107, or a combination thereof, and encoding the CAR-based promoter-therapeutic payload construct comprising the amino acid sequence of SEQ ID NO: 104, 106, 108, or a combination thereof, such that use of the dominant negative receptors dnTGFb and dnPD1 confers prevention of the auto-immunity toxicity typically associated with the use of constitutive activation of the dominant negative receptors, while simultaneously benefiting from the reduced immunosuppression of T cell function when the CAR is expressed and functional, or confers greater resistance to immunosuppression of CAR T cells by tumor microenvironment that each dn receptor alone, or a combination thereof.

In yet another embodiment, an isolated nucleic acid molecule is provided wherein the surface antigen-regulated inducible promoter-therapeutic payload construct comprises the nucleic acid sequence of SEQ ID NO: 103, and encodes the CAR LTG1563 with a dominant negative version of inhibitory TGF-beta receptor comprising the amino acid sequence as set forth in SEQ ID NO: 104, or an amino acid sequence with 85%, 90%, 95%, 96%, 97%, 98% or 99% identity thereof.

In yet another embodiment, an isolated nucleic acid molecule is provided wherein the surface antigen-regulated inducible promoter-therapeutic payload construct comprises the nucleic acid sequence of SEQ ID NO: 105, and encodes the CAR LTG1563 with a dominant negative version of PD1 comprising the amino acid sequence as set forth in SEQ ID NO: 106, or an amino acid sequence with 85%, 90%, 95%, 96%, 97%, 98% or 99% identity thereof.

In yet another embodiment, an isolated nucleic acid molecule is provided wherein the surface antigen-regulated inducible promoter-therapeutic payload construct with the AP1-NFκB_RE promoter with the dominant negative version of inhibitory TGF-beta receptor and the dominant negative version of PD1 co-expressed with the CAR LTG1563 construct, separated by ribosomal skipping sites (P2A) comprising the nucleic acid sequence of SEQ ID NO: 107 (the construct with the dominant negative version of inhibitory TGF-beta receptor and the dominant negative version of PD1 co-expressed with the CAR LTG1563 construct, separated by ribosomal skipping sites (P2A) and encodes the CAR comprising the amino acid sequence as set forth in SEQ ID NO: 108 or an amino acid sequence with 85%, 90%, 95%, 96%, 97%, 98% or 99% identity thereof.

In yet another embodiment, an isolated nucleic acid molecule is provided wherein the surface antigen-regulated inducible promoter-therapeutic payload construct with the STAT5_RE promoter comprises the nucleic acid sequence of SEQ ID NO: 109 and encodes the CAR LTG1563 with a dominant negative version of inhibitory TGF-beta comprising the amino acid sequence as set forth in SEQ ID NO: 104, or an amino acid sequence with 85%, 90%, 95%, 96%, 97%, 98% or 99% identity thereof.

In yet another embodiment, an isolated nucleic acid molecule is provided wherein the surface antigen-regulated inducible promoter-therapeutic payload construct with the STAT5_RE promoter comprises the nucleic acid sequence of SEQ ID NO: 110 and encodes the CAR LTG1563 with the dominant negative version of PD1 comprising the amino acid sequence as set forth in SEQ ID NO: 106, or an amino acid sequence with 85%, 90%, 95%, 96%, 97%, 98% or 99% identity thereof.

In another embodiment, an isolated nucleic acid molecule is provided wherein the surface antigen-regulated inducible promoter-therapeutic payload construct with the STAT5_RE promoter with the dominant negative version of inhibitory TGF-beta receptor and the dominant negative version of PD1 are co-expressed with the CAR LTG1563 construct, separated by ribosomal skipping sites (P2A) comprising the nucleic acid sequence of SEQ ID NO: 111 and encodes the CAR comprising the amino acid sequence as set forth in SEQ ID NO: 108 or an amino acid sequence with 85%, 90%, 95%, 96%, 97%, 98% or 99% identity thereof.

In yet another embodiment, an isolated nucleic acid molecule is provided wherein the constitutive promoter-therapeutic payload construct with the EF1a promoter with the dominant negative version of inhibitory TGF-beta receptor is co-expressed with the CAR LTG1563 construct, wherein the constitutive EF1a promoter is used to express the additional protein with the co-expressed CAR LTG1563 construct.

In yet another embodiment, an isolated nucleic acid molecule is provided wherein the constitutive promoter-therapeutic payload construct with the EF1a promoter with the dominant negative version of PD1 is co-expressed with the CAR LTG1563 construct, wherein the constitutive EF1a promoter is used to express the additional protein with the co-expressed CAR LTG1563 construct.

In yet another embodiment, an isolated nucleic acid molecule is provided wherein the constitutive promoter-therapeutic payload construct with the EF1a promoter with the dominant negative version of inhibitory TGF-beta receptor and the dominant negative version of PD1 is co-expressed with the CAR LTG1563 construct and are separated by ribosomal skipping sites (P2A) wherein the constitutive EF1a promoter is used to successfully express the additional proteins with the co-expressed CAR LTG1563 construct.

### 6. Additional Description of CARs

Also expressly included within the scope of the invention are functional portions of the surface antigen-regulated promoter-therapeutic payload constructs disclosed herein (e.g. CAR-, cytokine-, chemokine-, trafficking receptor-, bispecific antibody-, a neutralizing/blocking antibody-, a T cell stimulatory receptor-, a truncated inhibitory receptor-, a hybrid inhibitory/activating receptor-, an anti-apoptotic protein-, an shRNA-, a protease-based). The term “functional portion” when used in reference to a CAR refers to any part or fragment of one or more of the CARs disclosed herein, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion (e.g., recognize target cells, detect cancer, treat or prevent cancer, etc.). More desirably, the additional amino acids enhance the biological activity, as compared to the biological activity of the parent CAR.

Included in the scope of the disclosure are functional variants of the CARs disclosed herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, 50%, 75%, 80%, 90%, 98% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, He, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., He, Thr, and Val), an amino acid with an aromatic side-chain substituted for another amino acid with an aromatic side chain (e.g., His, Phe, Trp, and Tyr), etc.

The CAR can consist essentially of the specified amino acid sequence or sequences described herein, such that other components, e.g., other amino acids, do not materially change the biological activity of the functional variant.

The CARs (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs (including functional portions and functional variants of the invention) can comprise synthetic amino acids in place of one or more naturally-occurring amino acids. Such synthetic amino acids are known in the art, and include, for example, aminocyclohexane carboxylic acid, norleucine, -amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, -aminocyclopentane carboxylic acid, a-aminocyclohexane carboxylic acid, a-aminocycloheptane carboxylic acid, a-(2-amino-2-norbornane)-carboxylic acid, γ-diaminobutyric acid, β-diaminopropionic acid, homophenylalanine, and a-tert-butylglycine.

The CARs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs (including functional portions and functional variants thereof) can be obtained by methods known in the art. The CARs may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N Y 2001; and Ausubel et al., Current Protocols in Molecular Biology, Greene Publishing Associates and John Wiley & Sons, N Y, 1994. Further, some of the CARs (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies. In this respect, the CARs can be synthetic, recombinant, isolated, and/or purified.

In each of the aforementioned recitations in Section A above, in addition to the CAR-based surface antigen-regulated promoter-therapeutic payload constructs described supra, the surface antigen-regulated promoter-therapeutic payload constructs may further comprise various other therapeutic modalities or auxiliary components comprising one or more of the following: a cytokine comprising IL-2, IL-15, IL-7, TNFa, IFN gamma, IFN beta, IFN alpha, IL-21, IL-33, IL-22, IL-6, IL-10, IL-9, IL-4, IL-12, TGF beta, IL-17, IL-18, a chemokine comprising CCR4, CCR6, CXCR5, a trafficking receptor, such as a cytokine receptor, for example, and not by way of limitation, CCR4, CCR7, CCR2, a bispecific antibody, including a Bi-specific T cell engager (BiTE), for example, and not by way of limitation, anti-CD3 and anti CD19 targeting, or other multi-targeting antibody, a neutralizing/blocking antibody, for example, and not by way of limitation, against PD-L1, IL-6R, IL-1R, expressed as an scFv, or as IgG, or in other configurations, a T cell stimulatory receptor, a truncated inhibitory receptor, a hybrid inhibitory/activating receptor, for example, and not by way of limitation, ectodomain of PD-1, fused to endodomain of CD28, an anti-apoptotic protein, for example, and not by way of limitation, BCL-2, or BCL-XL, an shRNA, a protease, a second CAR T construct, or any combination thereof, each with their aforementioned biological properties as listed supra.

### D. Antibodies and Antigen Binding Fragments

One embodiment further provides a CAR-based surface antigen-regulated promoter-therapeutic payload construct, for example and not by way of limitation, a T-cell expressing a CAR, an antibody, or antigen binding domain or portion thereof, which specifically binds to one or more of the antigens disclosed herein. As used herein, a “T-cell expressing a CAR,” or a “CAR T cell” means a T-cell expressing a CAR, and has antigen specificity determined by, for example, the antibody-derived targeting domain of the CAR.

As used herein, “antigen binding domain” can include an antibody and antigen binding fragments thereof. The term “antibody” is used herein in the broadest sense and encompasses various antibody structures, including but not limited to monoclonal antibodies, polyclonal antibodies, multi-specific antibodies (e.g., bispecific antibodies), and antigen binding fragments thereof, so long as they exhibit the desired antigen-binding activity. Non-limiting examples of antibodies include, for example, intact immunoglobulins and variants and fragments thereof known in the art that retain binding affinity for the antigen.

A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. In some examples, a monoclonal antibody is an antibody produced by a single clone of B lymphocytes or by a cell into which nucleic acid encoding the light and heavy variable regions of the antibody of a single antibody (or an antigen binding fragment thereof) have been transfected, or a progeny thereof. In some examples monoclonal antibodies are isolated from a subject. Monoclonal antibodies can have conservative amino acid substitutions which have substantially no effect on antigen binding or other immunoglobulin functions. Exemplary methods of production of monoclonal antibodies are known, for example, see Harlow & Lane, Antibodies, A Laboratory Manual, 2nd ed. Cold Spring Harbor Publications, New York (2013).

Typically, an immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain; see, e.g., Kindt et al. Kuby Immunology, 6^{th} ed., W.H. Freeman and Co., page 91 (2007).) In several embodiments, the heavy and the light chain variable regions combine to specifically bind the antigen. In additional embodiments, only the heavy chain variable region is required. For example, naturally occurring camelid antibodies consisting of a heavy chain only are functional and stable in the absence of light chain (see, e.g., Hamers-Casterman et al., Nature, 363:446-448, 1993; Sheriff et al., Nat. Struct. Biol., 3:733-736, 1996). References to “VH” or “VH” refer to the variable region of an antibody heavy chain, including that of an antigen binding fragment, such as Fv, ScFv, dsFv or Fab. References to “VL” or “VL” refer to the variable domain of an antibody light chain, including that of an Fv, ScFv, dsFv or Fab.

Light and heavy chain variable regions contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (“Sequences of Proteins of Immunological Interest,” 5^{th} Ed. Public Health Service, National Institutes of Health, Bethesda, MD, 1991; “Kabat” numbering scheme), Al-Lazikani et al., (JMB 273,927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27:55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a VH CDR3 is the CDR3 from the variable domain of the heavy chain of the antibody in which it is found, whereas a VL CDR1 is the CDR1 from the variable domain of the light chain of the antibody in which it is found. Light chain CDRs are sometimes referred to as LCDR1, LCDR2, and LCDR3. Heavy chain CDRs are sometimes referred to as HCDR1, HCDR2, and HCDR3.

An “antigen binding fragment” is a portion of a full length antibody that retains the ability to specifically recognize the cognate antigen, as well as various combinations of such portions. Non-limiting examples of antigen binding fragments include Fv, Fab, Fab′, Fab′-SH, F(ab′)2; diabodies; linear antibodies; single-chain antibody molecules (e.g. ScFv); and multi-specific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2nd Ed., Springer Press, 2010).

A single-chain antibody (ScFv) is a genetically engineered molecule containing the VH and VL domains of one or more antibody(ies) linked by a suitable polypeptide linker as a genetically fused single chain molecule (see, for example, Bird et al., Science, 242:423 426, 1988; Huston et al., Proc. Natl. Acad. Sci., 85:5879 5883, 1988; Ahmad et al., Clin. Dev. Immunol., 2012, doi:10.1155/2012/980250; Marbry, IDrugs, 13:543-549, 2010). The intramolecular orientation of the VH-domain and the VL-domain in a ScFv, is typically not decisive for ScFvs. Thus, ScFvs with both possible arrangements (VH-domain-linker domain-VL-domain; VL-domain-linker domain-VH-domain) may be used.

In a dsFv, the heavy and light chain variable chains have been mutated to introduce a disulfide bond to stabilize the association of the chains. Diabodies also are included, which are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444 6448, 1993; Poljak et al., Structure, 2:1121 1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, IL); Kuby, J., Immunology, 3rd Ed., W.H. Freeman & Co., New York, 1997.

Non-naturally occurring antibodies can be constructed using solid phase peptide synthesis, can be produced recombinantly, or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies, are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995); each of which is incorporated herein by reference).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Antibody competition assays are known, and an exemplary competition assay is provided herein.

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1st Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Methods of testing antibodies for the ability to bind to any functional portion of the CAR are known in the art and include any antibody-antigen binding assay, such as, for example, radioimmunoassay (RIA), ELISA, Western blot, immunoprecipitation, and competitive inhibition assays (see, e.g., Janeway et al., infra, U.S. Patent Application Publication No. 2002/0197266 A1, and U.S. Pat. No. 7,338,929).

Also, a CAR, a T cell expressing a CAR, an antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

In each of the aforementioned recitation in Section B above, in addition to the CAR-based surface antigen-regulated promoter-therapeutic payload constructs described supra, the surface antigen-regulated promoter-therapeutic payload constructs may further comprise various other therapeutic modalities comprising one or more of the following: a cytokine comprising IL-2, IL-15, IL-7, TNFa, IFN gamma, IFN beta, IFN alpha, IL-21, IL-33, IL-22, IL-6, IL-10, IL-9, IL-4, IL-12, TGF beta, IL-17, IL-18, a chemokine comprising CCR4, CCR6, CXCR5, a trafficking receptor, such as a cytokine receptor, for example, and not by way of limitation, CCR4, CCR7, CCR2, a bispecific antibody, including a Bi-specific T cell engager (BiTE), for example, and not by way of limitation, anti-CD3 and anti CD19 targeting, or other multi-targeting antibody, a neutralizing/blocking antibody, for example, and not by way of limitation, against PD-L1, IL-6R, IL-1R, expressed as an scFv, or as IgG, or in other configurations, a T cell stimulatory receptor, a truncated inhibitory receptor, a hybrid inhibitory/activating receptor, for example, and not by way of limitation, ectodomain of PD-1, fused to endodomain of CD28, an anti-apoptotic protein, for example, and not by way of limitation, BCL-2, or BCL-XL, an shRNA, a protease, a second CAR T construct, or any combination thereof, each with their aforementioned biological properties as listed supra.

### E. Conjugates

A surface antigen-regulated promoter-therapeutic payload construct (e.g., CAR-, cytokine-, chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based), expressing, for example, a CAR or monoclonal antibodies, or antigen binding fragments thereof, specific for one or more of the antigens disclosed herein, can be conjugated to an agent, such as an effector molecule or detectable marker, using any number of means known to those of skill in the art. Both covalent and noncovalent attachment means may be used. Conjugates include, but are not limited to, molecules in which there is a covalent linkage of an effector molecule or a detectable marker to an antibody or antigen binding fragment that specifically binds one or more of the antigens disclosed herein. One of skill in the art will appreciate that various effector molecules and detectable markers can be used, including (but not limited to) chemotherapeutic agents, anti-angiogenic agents, toxins, radioactive agents such as ¹²⁵I, ³²P, ¹⁴C, ³H and ³⁵S and other labels, target moieties and ligands, etc.

The choice of a particular effector molecule or detectable marker depends on the particular target molecule or cell, and the desired biological effect. Thus, for example, the effector molecule can be a cytotoxin that is used to bring about the death of a particular target cell (such as a tumor cell).

The procedure for attaching an effector molecule or detectable marker to an antibody or antigen binding fragment varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule or detectable marker. Alternatively, the antibody or antigen binding fragment is derivatized to expose or attach additional reactive functional groups. The derivatization may involve attachment of any of a number of known linker molecules such as those available from Pierce Chemical Company, Rockford, IL. The linker can be any molecule used to join the antibody or antigen binding fragment to the effector molecule or detectable marker. The linker is capable of forming covalent bonds to both the antibody or antigen binding fragment and to the effector molecule or detectable marker. Suitable linkers are well known to those of skill in the art and include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side groups (such as through a disulfide linkage to cysteine) or to the alpha carbon amino and carboxyl groups of the terminal amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,5667, 498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

In some embodiments, the linker is cleavable under intracellular conditions, such that cleavage of the linker releases the effector molecule or detectable marker from the antibody or antigen binding fragment in the intracellular environment. In yet other embodiments, the linker is not cleavable and the effector molecule or detectable marker is released, for example, by antibody degradation. In some embodiments, the linker is cleavable by a cleaving agent that is present in the intracellular environment (for example, within a lysosome or endosome or caveolea). The linker can be, for example, a peptide linker that is cleaved by an intracellular peptidase or protease enzyme, including, but not limited to, a lysosomal or endosomal protease. In some embodiments, the peptide linker is at least two amino acids long or at least three amino acids long. However, the linker can be 4, 5, 6, 7, 8, 9, 10, 11, 12, 13, 14 or 15 amino acids long, such as 1-2, 1-3, 2-5, 3-10, 3-15, 1-5, 1-10, 1-15 amino acids long. Proteases can include cathepsins B and D and plasmin, all of which are known to hydrolyze dipeptide drug derivatives resulting in the release of active drug inside target cells (see, for example, Dubowchik and Walker, 1999, Pharm. Therapeutics 83:67-123). For example, a peptide linker that is cleavable by the thiol-dependent protease cathepsin-B, can be used (for example, a Phenylalanine-Leucine or a Glycine-Phenylalanine-Leucine-Glycine linker (SEQ ID NO: 186)). Other examples of such linkers are described, for example, in U.S. Pat. No. 6,214,345, incorporated herein by reference. In a specific embodiment, the peptide linker cleavable by an intracellular protease is a Valine-Citruline linker or a Phenylalanine-Lysine linker (see, for example, U.S. Pat. No. 6,214,345, which describes the synthesis of doxorubicin with the Valine-Citruline linker).

In other embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (for example, a hydrazone, semicarbazone, thiosemicarbazone, cis-aconitic amide, orthoester, acetal, ketal, or the like) can be used. (See, for example, U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, for example, a thioether attached to the therapeutic agent via an acylhydrazone bond (see, for example, U.S. Pat. No. 5,622,929).

In other embodiments, the linker is cleavable under reducing conditions (for example, a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT. (See, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker is not cleavable and the effector molecule or detectable marker is released by antibody degradation. (See U.S. Publication No. 2005/0238649 incorporated by reference herein in its entirety).

In several embodiments, the linker is resistant to cleavage in an extracellular environment. For example, no more than about 20%, no more than about 15%, no more than about 10%, no more than about 5%, no more than about 3%, or no more than about 1% of the linkers, in a sample of conjugate, are cleaved when the conjugate is present in an extracellular environment (for example, in plasma). Whether or not a linker is resistant to cleavage in an extracellular environment can be determined, for example, by incubating the conjugate containing the linker of interest with plasma for a predetermined time period (for example, 2, 4, 8, 16, or 24 hours) and then quantitating the amount of free effector molecule or detectable marker present in the plasma. A variety of exemplary linkers that can be used in conjugates are described in WO 2004-010957, U.S. Publication No. 2006/0074008, U.S. Publication No. 20050238649, and U.S. Publication No. 2006/0024317, each of which is incorporated by reference herein in its entirety.

In several embodiments, a surface antigen-regulated promoter-therapeutic payload construct conjugate (e.g., CAR-, cytokine-, chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based), expressing, for example, a CAR, an antibody, or antigen binding portion thereof, and one or more small molecule toxins, such as a calicheamicin, maytansinoids, dolastatins, auristatins, a trichothecene, and CC1065, and the derivatives of these toxins that have toxin activity, are provided.

Maytansine compounds suitable for use as maytansinoid toxin moieties are well known in the art, and can be isolated from natural sources according to known methods, produced using genetic engineering techniques (see Yu et al. (2002) PNAS 99:7968-7973), or maytansinol and maytansinol analogues prepared synthetically according to known methods. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, each of which is incorporated herein by reference. Conjugates containing maytansinoids, methods of making same, and their therapeutic use are disclosed, for example, in U.S. Pat. Nos. 5,208,020; 5,416,064; 6,441,163 and European Patent EP 0 425 235 B1, the disclosures of which are hereby expressly incorporated by reference.

Additional toxins can be employed with a surface antigen-regulated promoter-therapeutic payload construct conjugate (e.g., CAR-, cytokine-, chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based), expressing, for example CAR, a T cell expressing a CAR, an antibody, or antigen binding portion thereof. Exemplary toxins include Pseudomonas exotoxin (PE), ricin, abrin, diphtheria toxin and subunits thereof, ribotoxin, ribonuclease, saporin, and calicheamicin, as well as botulinum toxins A through F. These toxins are well known in the art and many are readily available from commercial sources (for example, Sigma Chemical Company, St. Louis, MO). Contemplated toxins also include variants of the toxins (see, for example, U.S. Pat. Nos. 5,079,163 and 4,689,401).

Saporin is a toxin derived from Saponaria officinalis that disrupts protein synthesis by inactivating the 60S portion of the ribosomal complex (Stirpe et al., Bio/Technology, 10:405-412, 1992). However, the toxin has no mechanism for specific entry into cells, and therefore requires conjugation to an antibody or antigen binding fragment that recognizes a cell-surface protein that is internalized in order to be efficiently taken up by cells.

Diphtheria toxin is isolated from Corynebacterium diphtheriae. Typically, diphtheria toxin for use in immunotoxins is mutated to reduce or to eliminate non-specific toxicity. A mutant known as CRM107, which has full enzymatic activity but markedly reduced non-specific toxicity, has been known since the 1970's (Laird and Groman, J. Virol. 19:220, 1976), and has been used in human clinical trials. See, U.S. Pat. Nos. 5,792,458 and 5,208,021.

Ricin is the lectin RCA60 from Ricinus communis (Castor bean). For examples of ricin, see, U.S. Pat. Nos. 5,079,163 and 4,689,401. Ricinus communis agglutinin (RCA) occurs in two forms designated RCA₆₀ and RCA₁₂₀ according to their molecular weights of approximately 65 and 120 kD, respectively (Nicholson & Blaustein, J. Biochim. Biophys. Acta 266:543, 1972). The A chain is responsible for inactivating protein synthesis and killing cells. The B chain binds ricin to cell-surface galactose residues and facilitates transport of the A chain into the cytosol (Olsnes et al., Nature 249:627-631, 1974 and U.S. Pat. No. 3,060,165).

Ribonucleases have also been conjugated to targeting molecules for use as immunotoxins (see Suzuki et al., Nat. Biotech. 17:265-70, 1999). Exemplary ribotoxins such as α-sarcin and restrictocin are discussed in, for example Rathore et al., Gene 190:31-5, 1997; and Goyal and Batra, Biochem. 345 Pt 2:247-54, 2000. Calicheamicins were first isolated from Micromonospora echinospora and are members of the enediyne antitumor antibiotic family that cause double strand breaks in DNA that lead to apoptosis (see, for example Lee et al., J. Antibiot. 42:1070-87, 1989). The drug is the toxic moiety of an immunotoxin in clinical trials (see, for example, Gillespie et al., Ann. Oncol. 11:735-41, 2000).

Abrin includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B chain (abrin-b) binds to D-galactose residues (see, Funatsu et al., Agr. Biol. Chem. 52:1095, 1988; and Olsnes, Methods Enzymol. 50:330-335, 1978).

A surface antigen-regulated promoter-therapeutic payload construct (e.g., CAR-, cytokine-chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based), expressing, for example, a CAR, monoclonal antibodies, antigen binding fragments thereof, specific for one or more of the antigens disclosed herein, can also be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, Green fluorescent protein (GFP), Yellow fluorescent protein (YFP). A CAR, a T cell expressing a CAR, an antibody, or antigen binding portion thereof, can also be conjugated with enzymes that are useful for detection, such as horseradish peroxidase, β-galactosidase, luciferase, alkaline phosphatase, glucose oxidase and the like. When a CAR, a T cell expressing a CAR, an antibody, or antigen binding portion thereof, is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. A CAR, a T cell expressing a CAR, an antibody, or antigen binding portion thereof, may also be conjugated with biotin, and detected through indirect measurement of avidin or streptavidin binding. It should be noted that the avidin itself can be conjugated with an enzyme or a fluorescent label.

A surface antigen-regulated promoter-therapeutic payload construct (e.g., CAR-, cytokine-, chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based), expressing, for example, a CAR, an antibody, or antigen binding portion thereof, may be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitopes recognized by a secondary reporter (such as leucine zipper pair sequences, binding sites for secondary antibodies, metal binding domains, epitope tags).

A surface antigen-regulated promoter-therapeutic payload construct (e.g., CAR-, cytokine-, chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based), expressing, for example, a CAR, an antibody, or antigen binding portion thereof, can also be conjugated with a radiolabeled amino acid. The radiolabel may be used for both diagnostic and therapeutic purposes. For instance, the radiolabel may be used to detect one or more of the antigens disclosed herein and antigen expressing cells by x-ray, emission spectra, or other diagnostic techniques. Further, the radiolabel may be used therapeutically as a toxin for treatment of tumors in a subject, for example for treatment of a neuroblastoma. Examples of labels for polypeptides include, but are not limited to, the following radioisotopes or radionucleotides: ³H, ¹⁴C, ¹⁵N, ³⁵S, ⁹⁰Y ⁹⁹Tc, ¹¹¹In, ¹²⁵I, ¹³¹I.

Means of detecting such detectable markers are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

In each of the aforementioned recitations in Section C above, in addition to the CAR-based surface antigen-regulated promoter-therapeutic payload conjugate constructs described supra, the surface antigen-regulated promoter-therapeutic payload conjugate constructs may further comprise various other therapeutic modalities comprising one or more of the following: a cytokine comprising IL-2, IL-15, IL-7, TNFa, IFN gamma, IFN beta, IFN alpha, IL-21, IL-33, IL-22, IL-6, IL-10, IL-9, IL-4, IL-12, TGF beta, IL-17, IL-18, a chemokine comprising CCR4, CCR6, CXCR5, a trafficking receptor, such as a cytokine receptor, for example, and not by way of limitation, CCR4, CCR7, CCR2, a bispecific antibody, including a Bi-specific T cell engager (BiTE), for example, and not by way of limitation, anti-CD3 and anti CD19 targeting, or other multi-targeting antibody, a neutralizing/blocking antibody, for example, and not by way of limitation, against PD-L1, IL-6R, IL-1R, expressed as an scFv, or as IgG, or in other configurations, a T cell stimulatory receptor, a truncated inhibitory receptor, a hybrid inhibitory/activating receptor, for example, and not by way of limitation, ectodomain of PD-1, fused to endodomain of CD28, an anti-apoptotic protein, for example, and not by way of limitation, BCL-2, or BCL-XL, an shRNA, a protease, a second CAR T construct, or any combination thereof, each with their aforementioned biological properties as listed supra.

### F. Nucleotides, Expression, Vectors, and Host Cells

Further provided by an embodiment of the invention is a nucleic acid comprising a nucleotide sequence encoding any of surface antigen-regulated promoter-therapeutic payload constructs (e.g., CAR-, cytokine-, chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based), an antibody, or antigen binding portion thereof, described herein (including functional portions and functional variants thereof). The nucleic acids of the invention may comprise a nucleotide sequence encoding any of the leader sequences, antigen binding domains, transmembrane domains, and/or intracellular T cell signaling domains described herein.

In some embodiments, the nucleotide sequence may be codon-modified. Without being bound to a particular theory, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

In an embodiment of the invention, the nucleic acid may comprise a codon-modified nucleotide sequence that encodes the antigen binding domain of the inventive CAR. In another embodiment of the invention, the nucleic acid may comprise a codon-modified nucleotide sequence that encodes any of the CARs described herein (including functional portions and functional variants thereof).

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Sambrook et al., supra. The nucleic acids can be constructed based on chemical synthesis and/or enzymatic ligation reactions using procedures known in the art. See, for example, Sambrook et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Integrated DNA Technologies (Coralville, IA, USA).

The nucleic acid can comprise any isolated or purified nucleotide sequence which encodes any of the CARs or functional portions or functional variants thereof. Alternatively, the nucleotide sequence can comprise a nucleotide sequence which is degenerate to any of the sequences or a combination of degenerate sequences.

An embodiment also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive CARs. It is generally appreciated that conditions can be rendered more stringent by the addition of increasing amounts of formamide.

Also provided is a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an embodiment, the nucleic acids can be incorporated into a recombinant expression vector. In this regard, an embodiment provides recombinant expression vectors comprising any of the nucleic acids. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors are not naturally-occurring as a whole.

However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA).

Bacteriophage vectors, such as λ\ddot{υ}TIO, λ\ddot{υ}TI 1, λZapII (Stratagene), EMBL4, and λNMI 149, also can be used. Examples of plant expression vectors include pBIO1, pBI101.2, pBHO1 0.3, pBI121 and pBIN19 (Clontech). Examples of animal expression vectors include pEUK-Cl, pMAM, and pMAMneo (Clontech). The recombinant expression vector may be a viral vector, e.g., a retroviral vector or a lentiviral vector. A lentiviral vector is a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include, for example, and not by way of limitation, the LENTIVECTOR® gene delivery technology from Oxford BioMedica plc, the LENTIMAX™ vector system from Lentigen and the like. Nonclinical types of lentiviral vectors are also available and would be known to one skilled in the art.

A number of transfection techniques are generally known in the art (see, e.g., Graham et al., Virology, 52: 456-467 (1973); Sambrook et al., supra; Davis et al., Basic Methods in Molecular Biology, Elsevier (1986); and Chu et al, Gene, 13: 97 (1981).

Transfection methods include calcium phosphate co-precipitation (see, e.g., Graham et al., supra), direct micro injection into cultured cells (see, e.g., Capecchi, Cell, 22: 479-488 (1980)), electroporation (see, e.g., Shigekawa et al., BioTechniques, 6: 742-751 (1988)), liposome mediated gene transfer (see, e.g., Mannino et al., BioTechniques, 6: 682-690 (1988)), lipid mediated transduction (see, e.g., Feigner et al., Proc. Natl. Acad. Sci. USA, 84: 7413-7417 (1987)), and nucleic acid delivery using high velocity microprojectiles (see, e.g., Klein et al, Nature, 327: 70-73 (1987)).

In an embodiment, the recombinant expression vectors can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

An embodiment further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant CAR, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th1 and Th2 cells, CD8⁺ T cells (e.g., cytotoxic T cells), tumor infiltrating cells, memory T cells, memory stem cells, i.e. Tscm, naive T cells, and the like. The T cell may be a CD8⁺ T cell or a CD4⁺ T cell.

In an embodiment, the surface antigen-regulated promoter-therapeutic payload constructs as described herein can be used in suitable non-T cells. Such cells are those with an immune-effector function, such as, for example, NK cells, and T-like cells generated from pluripotent stem cells.

Also provided by an embodiment is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

CARs (including functional portions and variants thereof), nucleic acids, recombinant expression vectors, host cells (including populations thereof), and antibodies (including antigen binding portions thereof), can be isolated and/or purified. For example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

In each of the aforementioned recitations in Section D above, in addition to the CAR-based surface antigen-regulated promoter-therapeutic payload construct nucleotides, expression, vectors, and host cells described supra, the surface antigen-regulated promoter-therapeutic payload constructs may further comprise various other therapeutic modalities comprising one or more of the following: a cytokine comprising IL-2, IL-15, IL-7, TNFa, IFN gamma, IFN beta, IFN alpha, IL-21, IL-33, IL-22, IL-6, IL-10, IL-9, IL-4, IL-12, TGF beta, IL-17, IL-18, a chemokine comprising CCR4, CCR6, CXCR5, a trafficking receptor, such as a cytokine receptor, for example, and not by way of limitation, CCR4, CCR7, CCR2, a bispecific antibody, including a Bi-specific T cell engager (BiTE), for example, and not by way of limitation, anti-CD3 and anti CD19 targeting, or other multi-targeting antibody, a neutralizing/blocking antibody, for example, and not by way of limitation, against PD-L1, IL-6R, IL-1R, expressed as an scFv, or as IgG, or in other configurations, a T cell stimulatory receptor, a truncated inhibitory receptor, a hybrid inhibitory/activating receptor, for example, and not by way of limitation, ectodomain of PD-1, fused to endodomain of CD28, an anti-apoptotic protein, for example, and not by way of limitation, BCL-2, or BCL-XL, an shRNA, a protease, a second CAR T construct, or any combination thereof, each with their aforementioned biological properties as listed supra.

### G. Methods of Treatment

It is contemplated that the surface antigen-regulated promoter-therapeutic payload constructs (e.g., CAR-, cytokine-, chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based) disclosed herein can be used in methods of treating or preventing a disease in a mammal. In this regard, an embodiment provides a method of treating or preventing cancer in a mammal, comprising administering to the mammal the CARs, the nucleic acids, the recombinant expression vectors, the host cells, the population of cells, the antibodies and/or the antigen binding portions thereof, and/or the pharmaceutical compositions in an amount effective to treat or prevent cancer in the mammal.

An embodiment further comprises lymphodepleting the mammal prior to administering the CARs disclosed herein. Examples of lymphodepletion include, but may not be limited to, nonmyeloablative lymphodepleting chemotherapy, myeloablative lymphodepleting chemotherapy, total body irradiation, etc.

For purposes of the methods, wherein host cells or populations of cells are administered, the cells can be cells that are allogeneic or autologous to the mammal. Preferably, the cells are autologous to the mammal. As used herein, allogeneic means any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically. As used herein, “autologous” means any material derived from the same individual to whom it is later to be re-introduced into the individual.

The mammal referred to herein can be any mammal. As used herein, the term “mammal” refers to any mammal, including, but not limited to, mammals of the order Rodentia, such as mice and hamsters, and mammals of the order Lagomorpha, such as rabbits. The mammals may be from the order Carnivora, including Felines (cats) and Canines (dogs). The mammals may be from the order Artiodactyla, including Bovines (cows) and Swine (pigs) or of the order Perissodactyla, including Equines (horses). The mammals may be of the order Primates, Ceboids, or Simoids (monkeys) or of the order Anthropoids (humans and apes). Preferably, the mammal is a human.

With respect to the methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods can provide any amount or any level of treatment or prevention of cancer in a mammal.

Furthermore, the treatment or prevention provided by the method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment provides a method of detecting the presence of cancer in a mammal, comprising: (a) contacting a sample comprising one or more cells from the mammal with the CARs, the nucleic acids, the recombinant expression vectors, the host cells, the population of cells, the antibodies, and/or the antigen binding portions thereof, or the pharmaceutical compositions, thereby forming a complex, (b) and detecting the complex, wherein detection of the complex is indicative of the presence of cancer in the mammal.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer.

With respect to an embodiment of the method of detecting the presence of a proliferative disorder, e.g., cancer, in a mammal, the sample comprising cells of the mammal can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the mammal, e.g., the cells of any organ or tissue, including blood cells or endothelial cells.

The contacting can take place in vitro or in vivo with respect to the mammal. Preferably, the contacting is in vitro.

Also, detection of the complex can occur through any number of ways known in the art. For instance, the CARs disclosed herein, polypeptides, proteins, nucleic acids, recombinant expression vectors, host cells, populations of cells, or antibodies, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles) as disclosed supra.

Methods of testing a CAR for the ability to recognize target cells and for antigen specificity are known in the art. For instance, Clay et al., J. Immunol, 163: 507-513 (1999), teaches methods of measuring the release of cytokines (e.g., interferon-γ, granulocyte/monocyte colony stimulating factor (GM-CSF), tumor necrosis factor a (TNF-a) or interleukin 2 (IL-2)). In addition, CAR function can be evaluated by measurement of cellular cytotoxicity, as described in Zhao et al, J. Immunol, 174: 4415-4423 (2005).

Another embodiment provides for the use of the CARs, nucleic acids, recombinant expression vectors, host cells, populations of cells, antibodies, or antigen binding portions thereof, and/or pharmaceutical compositions of the invention, for the treatment or prevention of a proliferative disorder, e.g., cancer, in a mammal. The cancer may be any of the cancers described herein.

Any method of administration can be used for the disclosed therapeutic agents, including local and systemic administration. For example, topical, oral, intravascular such as intravenous, intramuscular, intraperitoneal, intranasal, intradermal, intrathecal and subcutaneous administration can be used. The particular mode of administration and the dosage regimen will be selected by the attending clinician, taking into account the particulars of the case (for example the subject, the disease, the disease state involved, and whether the treatment is prophylactic). In cases in which more than one agent or composition is being administered, one or more routes of administration may be used; for example, a chemotherapeutic agent may be administered orally and an antibody or antigen binding fragment or conjugate or composition may be administered intravenously. Methods of administration include injection for which the CAR, CAR T Cell, conjugates, antibodies, antigen binding fragments, or compositions are provided in a nontoxic pharmaceutically acceptable carrier such as water, saline, Ringer's solution, dextrose solution, 5% human serum albumin, fixed oils, ethyl oleate, or liposomes. In some embodiments, local administration of the disclosed compounds can be used, for instance by applying the antibody or antigen binding fragment to a region of tissue from which a tumor has been removed, or a region suspected of being prone to tumor development. In some embodiments, sustained intra-tumoral (or near-tumoral) release of the pharmaceutical preparation that includes a therapeutically effective amount of the antibody or antigen binding fragment may be beneficial. In other examples, the conjugate is applied as an eye drop topically to the cornea, or intravitreally into the eye.

The disclosed therapeutic agents can be formulated in unit dosage form suitable for individual administration of precise dosages. In addition, the disclosed therapeutic agents may be administered in a single dose or in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regime will also, at least in part, be determined based on the particular needs of the subject to be treated and will be dependent upon the judgment of the administering practitioner.

Typical dosages of the antibodies or conjugates can range from about 0.01 to about 30 mg/kg, such as from about 0.1 to about 10 mg/kg.

In particular examples, the subject is administered a therapeutic composition that includes one or more of the conjugates, antibodies, compositions, CARs, CAR T cells or additional agents, on a multiple daily dosing schedule, such as at least two consecutive days, 10 consecutive days, and so forth, for example for a period of weeks, months, or years. In one example, the subject is administered the conjugates, antibodies, compositions or additional agents for a period of at least 30 days, such as at least 2 months, at least 4 months, at least 6 months, at least 12 months, at least 24 months, or at least 36 months.

In some embodiments, the disclosed methods include providing surgery, radiation therapy, and/or chemotherapeutics to the subject in combination with a disclosed antibody, antigen binding fragment, conjugate, CAR or T cell expressing a CAR (for example, sequentially, substantially simultaneously, or simultaneously). Methods and therapeutic dosages of such agents and treatments are known to those skilled in the art, and can be determined by a skilled clinician. Preparation and dosing schedules for the additional agent may be used according to manufacturer's instructions or as determined empirically by the skilled practitioner. Preparation and dosing schedules for such chemotherapy are also described in Chemotherapy Service, (1992) Ed., M. C. Perry, Williams & Wilkins, Baltimore, MD.

In some embodiments, the combination therapy can include administration of a therapeutically effective amount of an additional cancer inhibitor to a subject. Non-limiting examples of additional therapeutic agents that can be used with the combination therapy include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. These agents (which are administered at a therapeutically effective amount) and treatments can be used alone or in combination. For example, any suitable anti-cancer or anti-angiogenic agent can be administered in combination with the CARS, CAR-T cells, antibodies, antigen binding fragment, or conjugates disclosed herein. Methods and therapeutic dosages of such agents are known to those skilled in the art, and can be determined by a skilled clinician.

Additional chemotherapeutic agents include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and tioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, rifampicin, hydroxyurea, and mitomycin; topoisomerase inhibitors, such as topotecan and irinotecan; monoclonal antibodies, such as alemtuzumab, bevacizumab, cetuximab, gemtuzumab, rituximab, panitumumab, pertuzumab, and trastuzumab; photosensitizers, such as aminolevulinic acid, methyl aminolevulinate, porfimer sodium, and verteporfin; and other agents, such as alitretinoin, altretamine, amsacrine, anagrelide, arsenic trioxide, asparaginase, axitinib, bexarotene, bevacizumab, bortezomib, celecoxib, denileukin diftitox, erlotinib, estramustine, gefitinib, hydroxycarbamide, imatinib, lapatinib, pazopanib, pentostatin, masoprocol, mitotane, pegaspargase, tamoxifen, sorafenib, sunitinib, vemurafinib, vandetanib, and tretinoin. Selection and therapeutic dosages of such agents are known to those skilled in the art, and can be determined by a skilled clinician.

The combination therapy may provide synergy and prove synergistic, that is, the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation, a synergistic effect may be attained when the compounds are administered or delivered sequentially, for example by different injections in separate syringes. In general, during alternation, an effective dosage of each active ingredient is administered sequentially, i.e. serially, whereas in combination therapy, effective dosages of two or more active ingredients are administered together.

In one embodiment, an effective amount of an antibody or antigen binding fragment that specifically binds to one or more of the antigens disclosed herein or a conjugate thereof is administered to a subject having a tumor following anti-cancer treatment. After a sufficient amount of time has elapsed to allow for the administered antibody or antigen binding fragment or conjugate to form an immune complex with the antigen expressed on the respective cancer cell, the immune complex is detected. The presence (or absence) of the immune complex indicates the effectiveness of the treatment. For example, an increase in the immune complex compared to a control taken prior to the treatment indicates that the treatment is not effective, whereas a decrease in the immune complex compared to a control taken prior to the treatment indicates that the treatment is effective.

In each of the aforementioned recitations in Section E above, in addition to the CAR-based surface antigen-regulated promoter-therapeutic payload construct methods of treatment described supra, the surface antigen-regulated promoter-therapeutic payload constructs may further comprise various other therapeutic modalities comprising one or more of the following: a cytokine comprising IL-2, IL-15, IL-7, TNFa, IFN gamma, IFN beta, IFN alpha, IL-21, IL-33, IL-22, IL-6, IL-10, IL-9, IL-4, IL-12, TGF beta, IL-17, IL-18, a chemokine comprising CCR4, CCR6, CXCR5, a trafficking receptor, such as a cytokine receptor, for example, and not by way of limitation, CCR4, CCR7, CCR2, a bispecific antibody, including a Bi-specific T cell engager (BiTE), for example, and not by way of limitation, anti-CD3 and anti CD19 targeting, or other multi-targeting antibody, a neutralizing/blocking antibody, for example, and not by way of limitation, against PD-L1, IL-6R, IL-1R, expressed as an scFv, or as IgG, or in other configurations, a T cell stimulatory receptor, a truncated inhibitory receptor, a hybrid inhibitory/activating receptor, for example, and not by way of limitation, ectodomain of PD-1, fused to endodomain of CD28, an anti-apoptotic protein, for example, and not by way of limitation, BCL-2, or BCL-XL, an shRNA, a protease, a second CAR T construct, or any combination thereof, each with their aforementioned biological properties as listed supra.

### H. Biopharmaceutical Compositions

Biopharmaceutical or biologics compositions (hereinafter, “compositions”) are provided herein for use in gene therapy, immunotherapy and/or cell therapy that include one or more of the disclosed surface antigen-regulated promoter-therapeutic payload constructs (e.g., CAR-, cytokine-, chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based), or T cells expressing a CAR, antibodies, antigen binding fragments, conjugates, CARs, or T cells expressing a CAR that specifically bind to one or more antigens disclosed herein, in a carrier (such as a pharmaceutically acceptable carrier). The compositions can be prepared in unit dosage forms for administration to a subject. The amount and timing of administration are at the discretion of the treating clinician to achieve the desired outcome. The compositions can be formulated for systemic (such as intravenous) or local (such as intra-tumor) administration. In one example, a disclosed CARs, or T cells expressing a CAR, antibody, antigen binding fragment, conjugate, is formulated for parenteral administration, such as intravenous administration. Compositions including a CAR, or T cell expressing a CAR, a conjugate, antibody or antigen binding fragment as disclosed herein are of use, for example, for the treatment and detection of a tumor, for example, and not by way of limitation, a neuroblastoma. In some examples, the compositions are useful for the treatment or detection of a carcinoma. The compositions including a CAR, or T cell expressing a CAR, a conjugate, antibody or antigen binding fragment as disclosed herein are also of use, for example, for the detection of pathological angiogenesis.

The compositions for administration can include a solution of the CAR, or T cell expressing a CAR, conjugate, antibody or antigen binding fragment dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents, adjuvant agents, and the like, for example, sodium acetate, sodium chloride, potassium chloride, calcium chloride, sodium lactate and the like. The concentration of a CAR, or T cell expressing a CAR, antibody or antigen binding fragment or conjugate in these formulations can vary widely, and will be selected primarily based on fluid volumes, viscosities, body weight and the like in accordance with the particular mode of administration selected and the subject's needs. Actual methods of preparing such dosage forms for use in in gene therapy, immunotherapy and/or cell therapy are known, or will be apparent, to those skilled in the art.

A typical composition for intravenous administration includes about 0.01 to about 30 mg/kg of antibody or antigen binding fragment or conjugate per subject per day (or the corresponding dose of a CAR, or T cell expressing a CAR, conjugate including the antibody or antigen binding fragment). Actual methods for preparing administrable compositions will be known or apparent to those skilled in the art and are described in more detail in such publications as Remington's Pharmaceutical Science, 19th ed., Mack Publishing Company, Easton, PA (1995).

A CAR, or T cell expressing a CAR, antibodies, antigen binding fragments, or conjugates may be provided in lyophilized form and rehydrated with sterile water before administration, although they are also provided in sterile solutions of known concentration. The CARs, or T cells expressing a CAR, antibody or antigen binding fragment or conjugate solution is then added to an infusion bag containing 0.9% sodium chloride, USP, and in some cases administered at a dosage of from 0.5 to 15 mg/kg of body weight. Considerable experience is available in the art in the administration of antibody or antigen binding fragment and conjugate drugs; for example, antibody drugs have been marketed in the U.S. since the approval of RITUXAN® in 1997. A CAR, or T cell expressing a CAR, antibodies, antigen binding fragments and conjugates thereof can be administered by slow infusion, rather than in an intravenous push or bolus. In one example, a higher loading dose is administered, with subsequent, maintenance doses being administered at a lower level. For example, an initial loading dose of 4 mg/kg antibody or antigen binding fragment (or the corresponding dose of a conjugate including the antibody or antigen binding fragment) may be infused over a period of some 90 minutes, followed by weekly maintenance doses for 4-8 weeks of 2 mg/kg infused over a 30 minute period if the previous dose was well tolerated.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include microspheres, microparticles, microcapsules, nanocapsules, nanospheres, and nanoparticles. Microcapsules contain the therapeutic protein, such as a cytotoxin or a drug, as a central core. In microspheres, the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the CARs, or T cells expressing a CAR, antibody or antigen binding fragment or conjugate compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

In each of the aforementioned recitations in Section F above, in addition to the CAR-based surface antigen-regulated promoter-therapeutic payload construct compositions described supra, the surface antigen-regulated promoter-therapeutic payload constructs may further comprise various other therapeutic modalities comprising one or more of the following: a cytokine comprising IL-2, IL-15, IL-7, TNFa, IFN gamma, IFN beta, IFN alpha, IL-21, IL-33, IL-22, IL-6, IL-10, IL-9, IL-4, IL-12, TGF beta, IL-17, IL-18, a chemokine comprising CCR4, CCR6, CXCR5, a trafficking receptor, such as a cytokine receptor, for example, and not by way of limitation, CCR4, CCR7, CCR2, a bispecific antibody, including a Bi-specific T cell engager (BiTE), for example, and not by way of limitation, anti-CD3 and anti CD19 targeting, or other multi-targeting antibody, a neutralizing/blocking antibody, for example, and not by way of limitation, against PD-L1, IL-6R, IL-1R, expressed as an scFv, or as IgG, or in other configurations, a T cell stimulatory receptor, a truncated inhibitory receptor, a hybrid inhibitory/activating receptor, for example, and not by way of limitation, ectodomain of PD-1, fused to endodomain of CD28, an anti-apoptotic protein, for example, and not by way of limitation, BCL-2, or BCL-XL, an shRNA, a protease, a second CAR T construct, or any combination thereof, each with their aforementioned biological properties as listed supra.

### I. Kits

In one aspect, kits employing the surface antigen-regulated promoter-therapeutic payload constructs (e.g., CAR-, cytokine-, chemokine-, trafficking receptor-, bispecific antibody-, a neutralizing/blocking antibody-, a T-cell stimulatory receptor-, a truncated inhibitory receptor-, a hybrid inhibitory/activating receptor-, an anti-apoptotic protein-, an shRNA-, a protease-based) disclosed herein are also provided. For example, kits for treating a tumor in a subject, or making a CAR T cell that expresses one or more of the CARs disclosed herein. The kits will typically include a disclosed antibody, antigen binding fragment, conjugate, nucleic acid molecule, CAR or T cell expressing a CAR as disclosed herein. More than one of the disclosed antibodies, antigen binding fragments, conjugates, nucleic acid molecules, CARs or T cells expressing a CAR can be included in the kit.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed antibodies, antigen binding fragments, conjugates, nucleic acid molecules, CARs or T cells expressing a CAR. In several embodiments the container may have a sterile access port (for example the container may be an intravenous solution bag or a vial having a stopper pierceable by a hypodermic injection needle). A label or package insert indicates that the composition is used for treating the particular condition.

The label or package insert typically will further include instructions for use of a disclosed antibodies, antigen binding fragments, conjugates, nucleic acid molecules, CARs or T cells expressing a CAR, for example, in a method of treating or preventing a tumor or of making a CAR T cell. The package insert typically includes instructions customarily included in commercial packages of therapeutic products that contain information about the indications, usage, dosage, administration, contraindications and/or warnings concerning the use of such therapeutic products. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In each of the aforementioned recitations in Section G above, in addition to the CAR-based surface antigen-regulated promoter-therapeutic payload constructs described supra, the surface antigen-regulated promoter-therapeutic payload constructs may further comprise various other therapeutic modalities comprising one or more of the following: a cytokine comprising IL-2, IL-15, IL-7, TNFa, IFN gamma, IFN beta, IFN alpha, IL-21, IL-33, IL-22, IL-6, IL-10, IL-9, IL-4, IL-12, TGF beta, IL-17, IL-18, a chemokine comprising CCR4, CCR6, CXCR5, a trafficking receptor, such as a cytokine receptor, for example, and not by way of limitation, CCR4, CCR7, CCR2, a bispecific antibody, including a Bi-specific T cell engager (BiTE), for example, and not by way of limitation, anti-CD3 and anti CD19 targeting, or other multi-targeting antibody, a neutralizing/blocking antibody, for example, and not by way of limitation, against PD-L1, IL-6R, IL-1R, expressed as an scFv, or as IgG, or in other configurations, a T cell stimulatory receptor, a truncated inhibitory receptor, a hybrid inhibitory/activating receptor, for example, and not by way of limitation, ectodomain of PD-1, fused to endodomain of CD28, an anti-apoptotic protein, for example, and not by way of limitation, BCL-2, or BCL-XL, an shRNA, a protease, a second CAR T construct, or any combination thereof, each with their aforementioned biological properties as listed supra.

### EXAMPLES

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### Example 1

### Generation of a Self-Driving Car Construct Targeting the CD19 Antigen

Despite clinical success of anti-CD19-based (e.g. CAR LTG1563) cancer therapy, suboptimal CAR activation and persistence on the one hand, and CAR over-activation and the associated toxicity (CRS, neurotoxicity) on the other hand, present a problem. In order to improve the safety and efficacy of CAR T therapy, a self-driving CAR was created.

In this Example, CAR T cells are described that utilize CAR signaling to regulate its own expression and the expression of co-regulated transgenes via two mechanisms, and the use of these mechanisms beyond CAR expression:

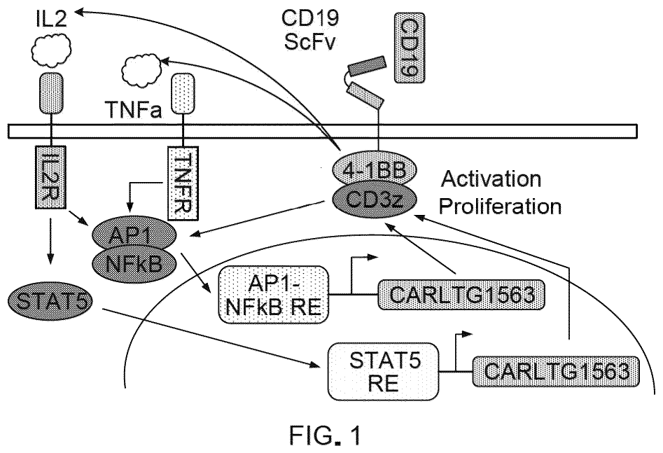

1. Positive regulation of CAR T expression by cytokine (e.g. IL-2/IL-15/IL-7)-driven STAT5 response element (STAT5_RE), which induce the expression of a CAR molecule (e.g. anti-CD19 CAR LTG1563). When CAR T detects tumor antigen, a slow positive feedback-loop is activated which increases CAR expression on CAR T cells. Once the tumors have been eliminated, the positive feedback loop gradually diminishes and shuts down, naturally reducing CAR surface expression and bringing it back to surveillance state (FIG. 1).

2. Positive regulation of CAR T expression by a combination of cytokine (e.g. IL-2/IL-15/IL-7/TNFα)-driven and CAR/TCR-driven AP1/NFκB response elements (AP1/NFκB_RE), which induce the expression of a CAR molecule (e.g. CAR LTG1563). When CAR T detects tumor antigen, a rapid positive feedback-loop is activated which increases CAR expression on CAR-T cells. Over time, the AP1/NFκB-driven CAR LTG1563 demonstrates greater CD19-dependent killing activity, increased cytokine secretion, decreased exhaustion, and overall fitness of CAR T cells relative to constitutively-expressed CARs. Once the tumors have been eliminated, the positive feedback loop gradually diminishes and shuts down, naturally reducing CAR surface expression and bringing it back to surveillance state (FIG. 1).

3. Expression of additional proteins in the presence of CAR antigens to modulate CAR T function, including but not limited to: evasion of negative T cell regulation (dominant negative TGFBRII receptor, anti-PD1 antibody, etc.), positive regulators of T cell growth (IL15, IL12, etc.), T cell homing to tumors (chemokine receptors), and greater access to the tumor microenvironment (matrix metalloproteinases), or one constitutive CAR and a second inducible CAR, such as both CARs are encoded by the same lentiviral vector, and such as the inducible CAR is expressed as a result of the activation of the constitutive CAR. The second CAR may be targeting a second tumor antigen, or an antigen expressed on myeloid-derived suppressor cells (MDSC) (e.g. CD33, mesothelin), or on inhibitory B cells (e.g., CD19). In each case, these inducible proteins will be expressed in the presence of CAR stimulation and downregulated following termination of CAR signaling.

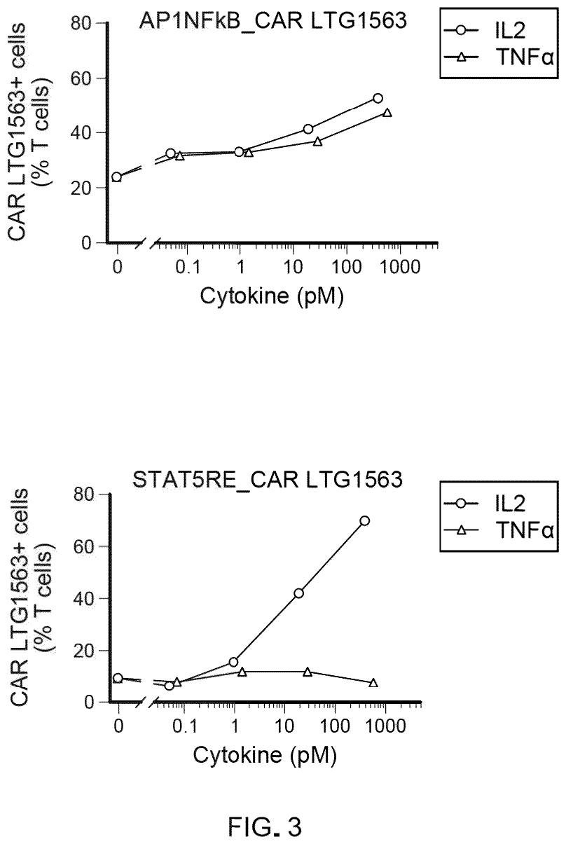

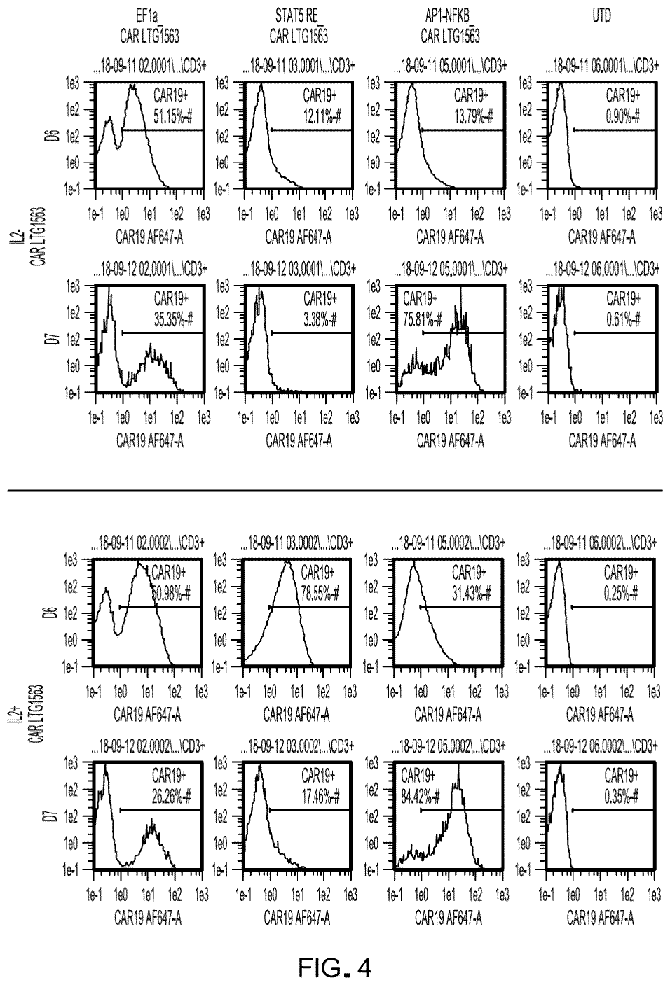

4. The STAT5_RE versus the AP1/NFκB_RE promoters can be used to modulate either self-expression of CAR proteins, or T cell function modulator proteins, in defined fashions. The greater cytokine (IL2)-dependent response of the STAT5_RE (FIG. 3) can be used to express proteins in CAR-T cells not directly responding to antigen, in a paracrine fashion near tumors. Alternatively, proteins can be expressed specifically in CAR T cells responding to tumors via AP1/NFκB_RE, due to the requirement for CAR signaling for the maximal response through this element (FIG. 4).

### Materials and Methods

### Creation of Chimeric Antigen Receptor (CAR)—Expressing Vectors

In order to generate the novel transmembrane domain of CAR LTG1563, the single chain variable fragment (scFv) derived from FMC-63 mouse hybridoma (FMC-63: AA 1-267, GenBank ID: HM852952.1) in orientation VL to VH and connected by the (GGGGS)3 flexible intrachain linker (SEQ ID NO: 187) was used. The resulting targeting domain was then linked in frame to human CD8 hinge (AA 138-179, Ref sequence ID NP_001759.3), human TNF receptor Superfamily 19 transmembrane domain (TNFRSF19, AA 167-196, UntProt sequence ID: Q9NS68), human 4-1BB co-stimulatory domain (CD137, AA 214-255, UniProt sequence ID: Q07011) and human CD3 zeta signaling domain (CD247, AA 52-163, Ref sequence ID: NP_000725.1.). Leader sequence from human granulocyte macrophage colony stimulating factor receptor alpha subunit (AA 1-22, GenBank ID: EAW98673.1) was included to facilitate CAR expression at cell surface. CAR sequence was codon-optimized and cloned into a third generation lentiviral plasmid backbone under the regulation of surface antigen-regulated inducible promoters described below, or constitutive MSCV or EF1α promoter as a control.

### Cell Lines Used to Demonstrate CAR Activity

The Burkitt lymphoma cell line Raji cell line, the chronic myelogenous leukemia line K562 line and reagents were purchased from American Tissue Culture Collection (ATCC, Manassass, VA), unless otherwise noted. Cells were cultured in RPMI-1640 medium supplemented with 10% heat-inactivated fetal bovine serum (FBS, Hyclone, Logan, UT) and 2 mM L-Glutamax (Thermo Fisher Scientific, Grand Island, NY). Human Embryonic kidney line 293T was purchased from ATCC and propagated in CD FortiCho medium (Gibco/Thermo Fisher Scientific, Grand Island, NY). Single-cell clones of luciferase-expressing cell lines were generated by stably transducing wild-type tumor lines with lentiviral vector encoding firefly luciferase (Lentigen Technology, Inc., Gaithersburg, MD), followed by cloning and selection of luciferase-positive clones.

### Primary Human T Cells Used to Demonstrate CAR Activity

Whole blood was collected from healthy volunteers at Oklahoma Blood Institute (OBI) with donors' written consent. Processed buffy coats were purchased from OBI (Oklahoma City, OK). The CD4-positive and CD8-positive human T cells were purified from buffy coats via positive selection using a 1:1 mixture of CD4- and CD8-MicroBeads (Miltenyi Biotec, Bergisch Gladbach, Germany) according to manufacturer's protocol.

### Primary T Cell Transduction

Human primary CD4+ and CD8+ T cells from normal donors were cultivated in TexMACS medium at a density of 1×106 cells/ml, activated with CD3/CD28 MACS® GMP T Cell TransAct reagent on day 0 (all reagents from Miltenyi Biotec), and transduced on day 1/2 with LV encoding CAR constructs overnight, and media exchanged on day 2/3. Supplementation with cytokines (IL-2, TNFα; Miltenyi Biotec, Bergisch Gladbach, Germany) was performed as described. Cultures were propagated until harvest on day 5-6 for co-incubation analysis.

### Immune Effector Assays (CTL and Cytokine)

For long-term co-incubation assays, CAR T effector and GFP-expressing target cells were propagated as above, and flow cytometric analysis was utilized to determine the extent of target cell population killing, and CAR T population survival and expansion. Cells were gated based on forward and side scatter, and viable (7AAD-negative) cells. Percentages of surviving cells post in co-cultures were determined based on GFP positivity for Raji targets and CD3 for CAR T effectors. In addition, CAR T expression in live CD3 positive cells was determined by staining with CD19 Fc peptide, followed by anti-Fc (Fab′)2—FL reagent.

### Results

Fully human CAR T constructs targeting the CD19 antigen were designed by combining in frame the sequences of leader peptide derived from GMCSFR, fully human anti-human CD19 ScFv sequence, CD8 hinge, TNFRSF19 transmembrane domain, 4-1BB costimulatory domain and CD3z activation domain.

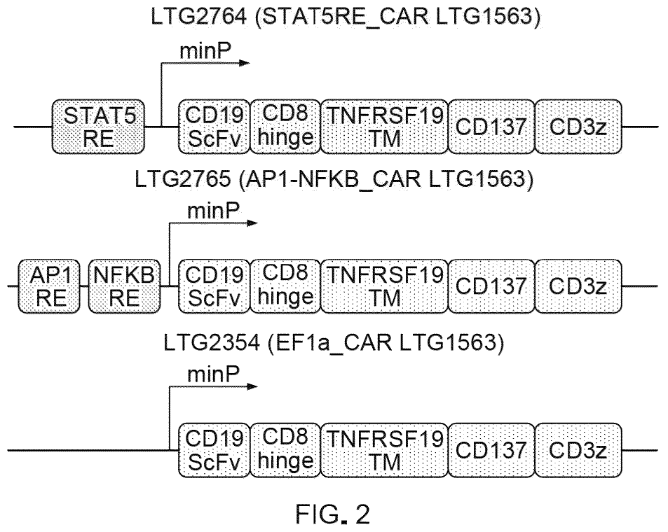

CAR positive regulation loop was designed by placing CAR LTG1563 expression under the control of a tandem STAT5 response element followed by a minimal promoter sequence. When IL-2 binds native IL-2 receptor on T cell surface, Jak/STAT signaling is initiated, STAT 5 translocates to the CAR promoter's regulatory region and enhances CAR expression. Constitutively-expressed EF1α-promoter driven CAR LTG1563 was constructed as a control and tested in parallel with STAT-5 and AP1/NFκB regulated CARs. Schema for positive CAR regulation via (1) IL-2 driven Stat5 and (2) IL-2/TNFα and CAR-driven AP1/NFκB-mediated mechanisms are depicted in FIG. 1, and the architecture of each of these constructs is depicted in FIG. 2.

T cells were purified from blood of two unrelated healthy donors by immunogenic selection using a 1:1 mixture of CD4 and CD8 beads (Miltenyi Biotec). Cells were transduced with CAR LTG1563 constructs in the absence of cytokine supplementation, as described in the materials and methods. The experimental groups included CD19 CAR driven by EF1a promoter at MOI 10, or with STAT5_RE CAR LTG1563 and AP1/NFκB_RE CAR LTG1563, positively regulated self-driving CARs at 0.25% vol/vol LV preparation (FIG. 3). Untransduced cells (UTD) cultured under same conditions were used as a negative control. On day 5, select groups received IL-2 or TNFα supplementation, and 20 h later CAR expression was detected by flow cytometry. EF1α-driven CAR expression was 30-40% depending on transducing MOI, and did not change due to IL-2/TNFα supplementation. By contrast, STAT5_RE CAR expression was greatly enhanced by IL-2 supplementation, but not by TNFα, as expected (FIG. 3). AP1/NFκB_RE CAR expression was enhanced by both IL-2 and TNFα, but to a lesser extent than IL-2 driven STAT5_RE CAR LTG1563 expression.

Further, in this experiment, the self-driving characteristic of both the STAT5 and AP1/NFκB response elements to drive CAR LTG1563 expression were assessed by CD19 antigen stimulation (FIG. 4). This was performed using CAR T cells from Donor A and Donor B in coculture with CD19 positive Raji NHL tumor cells stably expressing GFP. Strikingly, CAR expression was strongly induced in the AP1/NFκB_RE-driven CAR LTG1563 T cells within 20 h post-stimulation (FIG. 4B), demonstrating the integration of both CAR signaling and CAR-dependent cytokine expression when these response elements were used to drive CAR expression. The stimulation was much greater than could be observed simply by stimulating these cells with cytokines, indicating a stronger direct response to CAR signaling. This rapid AP1/NFκB-dependent CAR upregulation was in marked contrast to the constitutively-expressing EF1α-driven CAR LTG1563, which was transiently decreased by CD19 antigen-mediated stimulation of the CAR (likely due to internalization of these receptors). By contrast, the STAT5_RE-driven CAR LTG1563 was induced by coculture with CD19+ Raji cells, but on a much longer time scale than that observed for the AP1/NFκB-driven CAR LTG1563. This likely reflects an intermediate step required for the functionality of the STAT5 response element, the production of IL2 by CAR T cells and concomitant STAT5 signaling. IL2 supplementation of these cells from D3-D5 post-activation greatly increased the induction of CAR LTG1563 expression through the STAT5 element, likely by priming STAT-mediated signaling.

Notably, the expression of the AP1-NFκB CAR LTG1563 could be re-induced across multiple stimulations with CD19+ Raji cells (D6-D22 post-activation), indicating that the CAR T cells retain AP1/NFκB signaling responsiveness even during prolonged ex vivo cultures (data not shown). Importantly, the degree of CAR expression under the control of the AP1/NFκB promoter correlated quite well temporally with the number of Raji cells in the cocultures (data not shown). These results indicate that the AP1/NFκB element rapidly drives CAR LTG1563 expression in the presence of the cognate CD19 antigen, and that CAR LTG1563 is rapidly downregulated in the absence of antigen.

In the next experiment, T cells were purified from blood of three unrelated healthy donors by immunogenic selection using a 1:1 mixture of CD4 and CD8 beads (Miltenyi Biotec). Cells were transduced at D1 post-activation with CAR LTG1563 constructs in the absence of cytokine supplementation, as described in the materials and methods. The experimental groups included CD19 CAR driven by constitutive EF1α-derived CAR, STAT5 RE CAR LTG1563, and AP1-NFκB CAR LTG1563 were transduced with LV at an MOI of 10 (FIG. 3). In a subset of samples, IL2 supplementation (30 IU/mL) was carried out from D3-D6 post-activation, to prime expression of both the STAT5 RE CAR LTG1563 and AP1/NFκB CAR LTG1563. The CAR LTG1563-dependent cytotoxicity was assessed by coculture of CAR T cells with CD19+ Raji NHL cells stably expressing GFP. A very low effector to target ratio was used (1:3 CAR T:Raji cells), and CD19-dependent cytotoxicity was assessed by flow cytometric enumeration of the GFP+ Raji cells from D6-D13 post-activation (Coculture 1). Long-term function and expansion of the CAR T cells was assessed by re-stimulation of the cells at a similar effector to target ratio (1:3 Coculture 1:Raji cells) from D13-D20 post-activation (Coculture 2).

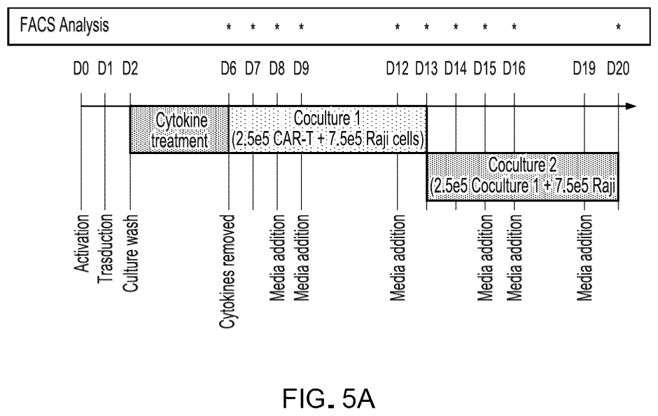

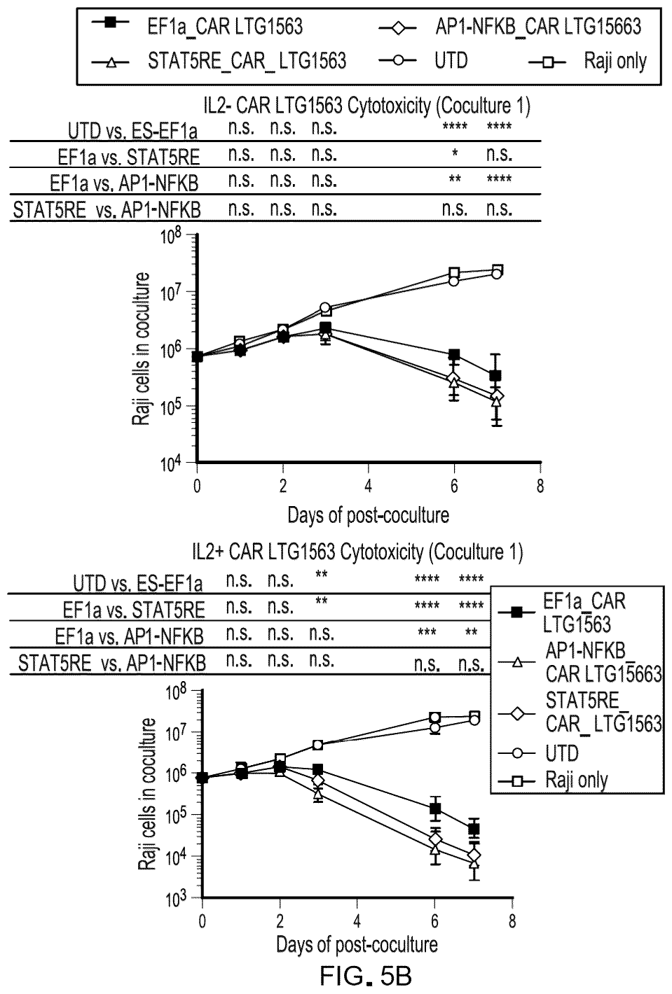

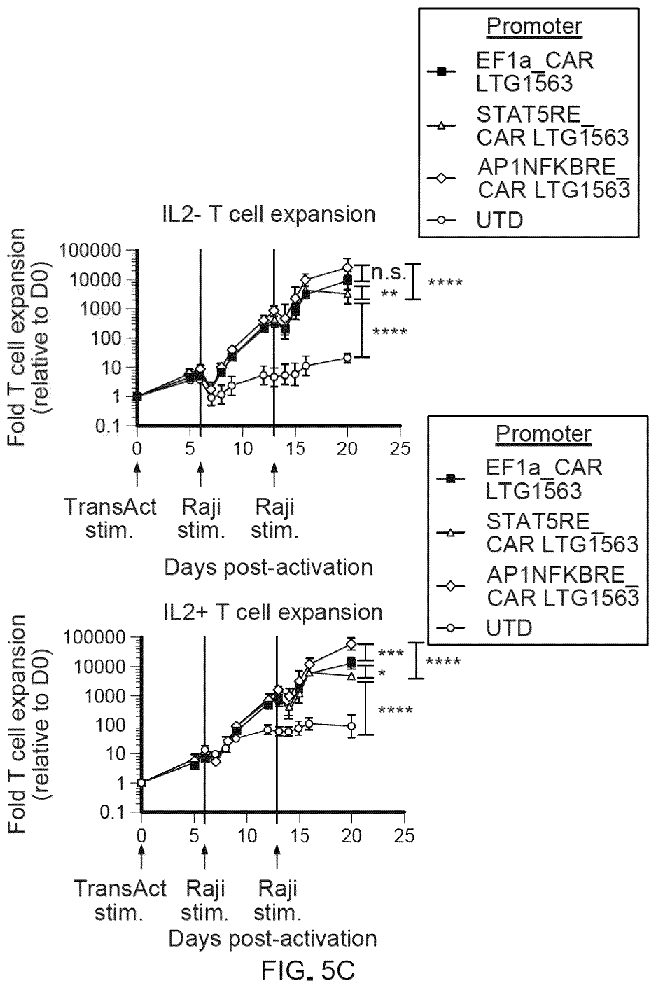

CAR T co-incubation with target cells was examined for a total of fourteen days (Coculture 1&2), during this time the numbers of viable Raji and T cells were enumerated by flow cytometric analysis (FIG. 5). Whereas Raji cells in Raji only and UTD control groups proceeded to grow unhindered to experimental day 8, CAR LTG1563 T cells strongly suppressed Raji expansion in all groups, regardless of IL2 priming. Of note, both STAT5 RE and AP1/NFκB CAR LTG1563 were superior to EF1α CAR LTG1563 in tumor killing throughout the initial coculture (Coculture 1), as measured on days 1, 2, 3, 6, and 7 after Raji addition (FIG. 5B). Strikingly, despite the low initial CAR LTG1563 expression in the IL2-cultures, both STAT5RE and AP1/NFκB outperformed the EF1a constitutively-expressing CAR LTG1563 construct (FIG. 5B). This potentially indicates that constitutive expression of CAR in the absence of a cognate antigen even for a short period (D2-D6 post-activation) may negatively affect the efficacy of CAR T cells, perhaps due to tonic signaling.

Further long-term analysis of CAR LTG1563-dependent cytotoxicity was assessed by re-stimulating the CAR T cells with CD19+ Raji cells. All CAR groups again demonstrated effective suppression of Raji expansion (data not shown), with AP1/NFκB-driven CAR LTG1563 showing superior tumor killing function as compared to constitutively-expressed EF1α_CAR LTG1563 (Figure. 5B). AP1/NFκB_CAR LTG1563-expressing T cells expanded to a greater extent throughout the culture period than either STATSRE_CAR LTG1563 or EF1α_CAR LTG1563-expressing T cells (FIG. 5C). This is likely due to CAR signaling being present only when target CD19+ cells are also present, preserving the overall health of these cells and leading to concomitantly greater T cell expansion.

Overall, the results demonstrate the superiority of inducible, self-driving AP1/NFκB RE CAR LTG1563, which was manifested in lower unstimulated CAR expression, and lower susceptibility to exhaustion, low level priming by cytokine supplementation, and rapid upregulation and sustained expression in the presence of tumor Raji cells. Additionally, superior CTL function was observed as compared to same CAR construct under the control of constitutive EF1α promoter in initial activity. This correlated with a greater overall expansion of these AP1/NFκB CAR T cells due to antigen stimulation relative to all other tested constructs.

### Example 2

### Extension of Self-Driving Expression to Auxiliary Proteins

Given the success of the inducible AP1/NFκB promoter in self-driving the high-level CAR LTG1563 expression specifically in the presence of the cognate CAR antigen (CD19), it was desired to extend the functionality of this promoter to auxiliary proteins, which provide additional functionality to CAR-T cells. The advantage of the CAR antigen-inducible AP1/NFκB promoter is that any proteins under the control of this promoter will be highly expressed only when the CAR tumor antigen is present. Therefore, the AP1/NFκB promoter was used to express proteins that allow CAR-T cells to escape immunosuppression, e.g., dominant-negative TGF-beta receptor (TGFBRIIdn) and dominant-negative programmed cell death protein 1 (PD1dn). These dominant-negative receptors were expected to allow the CAR-T cells to evade immunosuppression by either TGF-β or PD-L1, respectively. The expected advantage of using the AP1/NFκB promoter is that TGF-β/PD-L1 immunosuppression plays an important role under normal conditions in patients to prevent development of T cell-mediated autoimmunity. However, the microenvironment of many cancers are characterized by overexpression of TGF-β and/or PD-L1, which can prevent T cell or CAR-T cell-mediated anti-tumor immune responses. Therefore, it would be highly advantageous to express these dominant negative receptors specifically under conditions where tumor cells are present.

### Materials and Methods

### Creation of Dominant Negative Receptor(s)—Expressing Vectors

In order to generate the dominant negative receptors TGFBRIIdn and PD1dn, the proteins were linked in-frame to the CAR LTG1563-expressing vector (LTG1563). Downstream of the CAR LTG1563 sequence, a cleaved ribosome skip site (FP2AF) was introduced consisting of: a consensus furin cleavage site (amino acids: RAKR (SEQ ID NO: 188)) fused to a ribosome skip site (P2A) derived from the porcine teschovirus-1 polyprotein (AA 976-997, GenBank ID: CAB40546.1, mutated residue P977S) fused to a consensus furin cleavage sequence (amino acids: RAKR (SEQ ID NO: 188)). In TGFBRIIdn single-expressing vectors (LTG2864 and LTG2867), the resulting CAR LTG1563-FP2AF sequence was co-expressed with a dominant-negative TGF-beta receptor II sequence (TGFBRII: AA 1-191, Uniprot ID: P37173). In PD1dn single-expressing vectors (LTG2864 and LTG2867), the resulting CAR LTG1563-FP2AF sequence was co-expressed with a dominant-negative PD1 receptor (PD1: AA 1-199, Uniprot ID: Q15116).

In CAR-FP2AF-PD1dn-P2AF-TGFBRIIdn expressing vectors, CAR LTG1563-FP2AF was co-expressed with a dominant-negative PD1 receptor (PD1: AA 1-199, Uniprot ID: Q15116), followed by a consensus furin cleavage sequence (amino acids: RAKR (SEQ ID NO: 188)) fused to a ribosome skip site (P2A) derived from the porcine teschovirus-1 polyprotein (AA 976-997, GenBank ID: CAB40546.1, mutated residue P977S), and which in turn was co-expressed with a dominant-negative TGF-beta receptor II sequence (TGFBRII: AA 1-191, Uniprot ID: P37173). Protein sequences were codon-optimized and cloned into a third generation lentiviral plasmid backbone under the regulation of surface antigen-regulated inducible promoters AP1/NFκB, or constitutive EF1α promoter as a control.

### Cell Lines Used to Demonstrate CAR Activity

The Burkitt lymphoma cell line Raji cell line, the chronic myelogenous leukemia line K562 line and reagents were purchased from American Tissue Culture Collection (ATCC, Manassass, VA), unless otherwise noted. Cells were cultured in RPMI-1640 medium supplemented with 10% heat-inactivated fetal bovine serum (FBS, Hyclone, Logan, UT) and 2 mM L-Glutamax (Thermo Fisher Scientific, Grand Island, NY). Human Embryonic kidney line 293T was purchased from ATCC and propagated in CD FortiCho medium (Gibco/Thermo Fisher Scientific, Grand Island, NY). Single-cell clones of luciferase-expressing cell lines were generated by stably transducing wild-type tumor lines with lentiviral vector encoding firefly luciferase (Lentigen Technology, Inc., Gaithersburg, MD), followed by cloning and selection of luciferase-positive clones.

### Primary Human T Cells Used to Demonstrate CAR Activity

Whole blood was collected from healthy volunteers at Oklahoma Blood Institute (OBI) with donors' written consent. Processed buffy coats were purchased from OBI (Oklahoma City, OK). The CD4-positive and CD8-positive human T cells were purified from buffy coats via positive selection using a 1:1 mixture of CD4- and CD8-MicroBeads (Miltenyi Biotec, Bergisch Gladbach, Germany) according to manufacturer's protocol.

### Primary T Cell Transduction

Human primary CD4+ and CD8+ T cells from normal donors were cultivated in TexMACS medium at a density of 1×106 cells/ml, activated with CD3/CD28 MACS® GMP T Cell TransAct reagent on day 0 (all reagents from Miltenyi Biotec), and transduced on day 1/2 with LV encoding CAR constructs overnight, and media exchanged on day 2/3. Supplementation with cytokines (IL-2, TGFβ; Miltenyi Biotec, Bergisch Gladbach, Germany) was performed as described. Cultures were propagated until harvest on day 5-6 for co-incubation analysis.

### Immune Effector Assays (CTL and Cytokine)

For long-term co-incubation assays, CAR T effector and GFP-expressing target cells were propagated as above, and flow cytometric analysis was utilized to determine the extent of target cell population killing, and CAR T population survival and expansion. Cells were gated based on forward and side scatter, and viable (7AAD-negative) cells. Percentages of surviving cells post in co-cultures were determined based on GFP positivity for Raji targets and CD3 for CAR T effectors. In addition, CAR-T expression in live CD3 positive cells was determined by staining with CD19 Fc peptide, followed by anti-Fc (Fab′)2—FL reagent. PD1 and TGFBRII expression was also determined (encompassing both the transgenes PD1dn and TGFBRIIdn as well as the native PD1 and TGFBRII proteins).

### Results

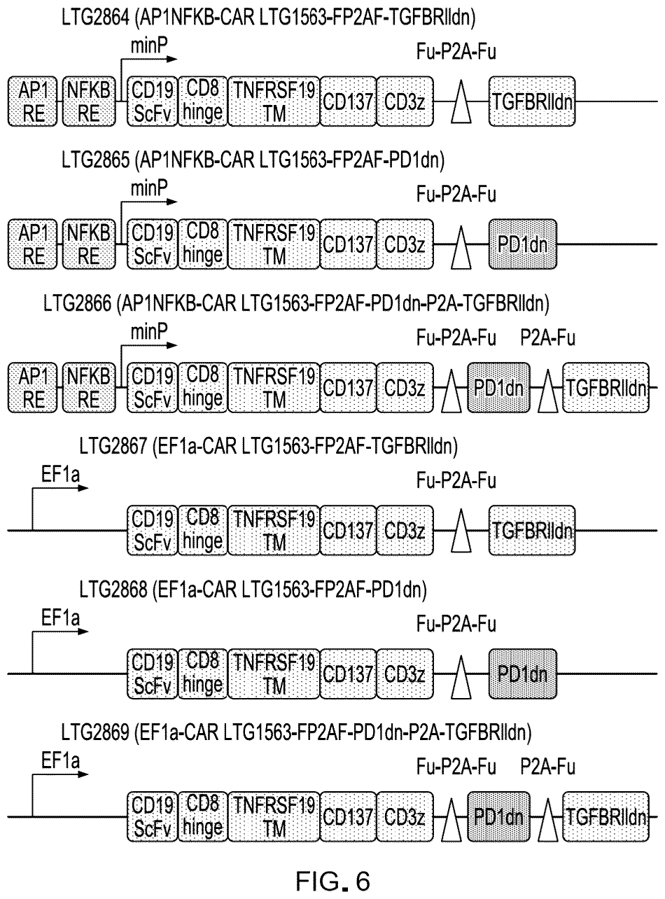

The self-driving capacity of the CAR LTG1563 constructs was extended to include auxiliary components to add additional functionality to the CAR-T cells, or CARs. In this case, dominant negative receptors lacking the intracellular signaling domains of either TGFBRII (TGFBRIIdn) or PD1 (PD1dn) were co-expressed with the CAR LTG1563 construct, via ribosomal skipping sites (P2A). Constitutively-expressed EF1α-promoter driven CAR LTG1563 was constructed as a control and tested in parallel with AP1/NFκB regulated CARs with auxiliary components. The architecture of each of these CAR constructs with auxiliary components is depicted in FIG. 6.

T cells were purified from blood of two unrelated healthy donors by immunogenic selection using a 1:1 mixture of CD4 and CD8 beads (Miltenyi Biotec). Cells were transduced with CAR LTG1563 constructs with auxiliary components in the absence of cytokine supplementation, as described in the materials and methods. The experimental groups included CAR LTG1563, CAR LTG1563-TGFBRIIdn, CAR LTG1563-PD1dn, or CAR LTG1563-PD1dn-TGFBRIIdn driven by the EF1a promoter, or with AP1/NFκB_RE-driven CAR LTG1563, CAR LTG1563-TGFBRIIdn, CAR LTG1563-PD1dn, or CAR LTG1563-PD1dn-TGFBRIIdn positively regulated self-driving CARs with auxiliary components at 0.5% vol/vol LV preparation (FIG. 6). Untransduced cells (UTD) cultured under same conditions were used as a negative control. The expression of CAR LTG1563 and the auxiliary components (TGFBRII, PD1) was assessed by flow cytometric enumeration; however, the expression of auxiliary components also reflects the expression of the native, full-length versions of these proteins on the CAR-T cells. Under the control of the constitutive EF1a promoter, a substantial increase in the expression of these TGFBRII and PD1 proteins was observed for the EF1a-CAR LTG1563-TGFBRIIdn and EF1a-CAR LTG1563-PD1dn constructs, respectively (FIG. 6B-6C), demonstrating that expression of the auxiliary transgenes was detectable above the native expression of these proteins.

Further, in this experiment, the self-driving characteristic of both the AP1/NFκB response elements to drive CAR LTG1563 expression were assessed by CD19 antigen stimulation (FIG. 6). This was performed using CAR T cells from Donor A and Donor B in coculture with CD19 positive Raji NHL tumor cells stably expressing GFP. Strikingly, CAR (FIG. 6A) and TGFBRII (FIG. 6B) expression was strongly induced in the AP1/NFκB_RE-CAR LTG1563-TGFBRIIdn T cells within 20 h post-stimulation. This indicates that the AP1/NFκB promoter can be used to induce proteins other than CARs in the presence of antigen signaling through a cognate CAR receptor.

Further, in this experiment, the CAR LTG1563-dependent cytotoxicity was assessed by coculture of CAR T cells with CD19+ Raji NHL cells stably expressing GFP, in the presence or absence of the immunosuppressive cytokine TGFβ (10 ng/mL). A very low effector to target ratio was used (1:3 CAR T:Raji cells), and CD19-dependent cytotoxicity was assessed by flow cytometric enumeration of the GFP+ Raji cells from D5-D9 post-activation (Coculture 1). Long-term function and expansion of the CAR T cells was assessed by a secondary re-stimulation of the cells at a similar effector to target ratio (1:3 Coculture 1:Raji cells) from D9-D13 post-activation (Coculture 2), and a tertiary re-stimulation (1:3 Coculture 2:Raji cells) from D13-D19 post-activation.

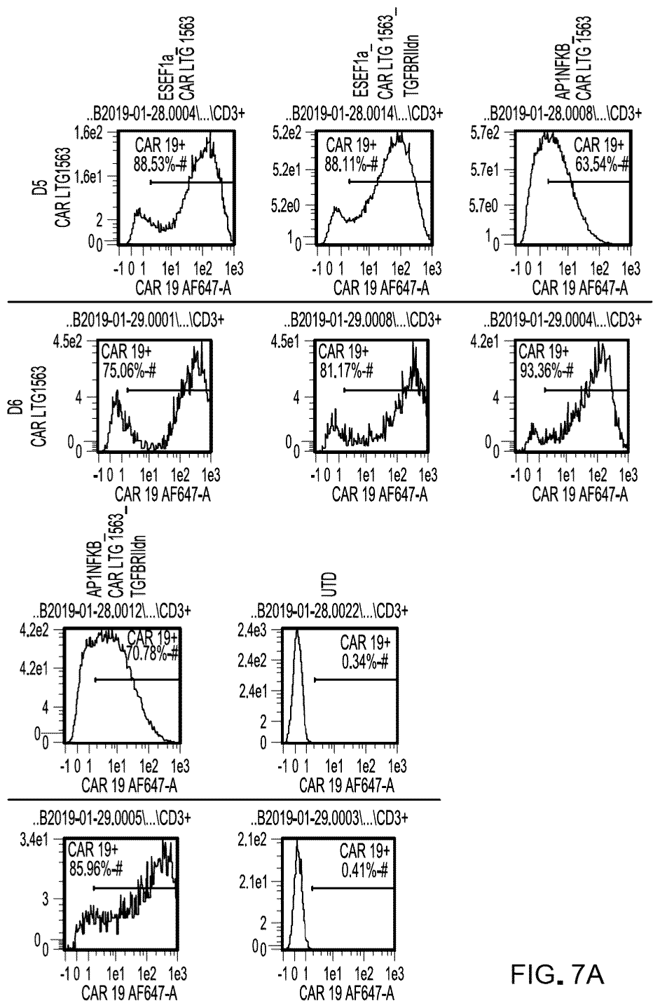

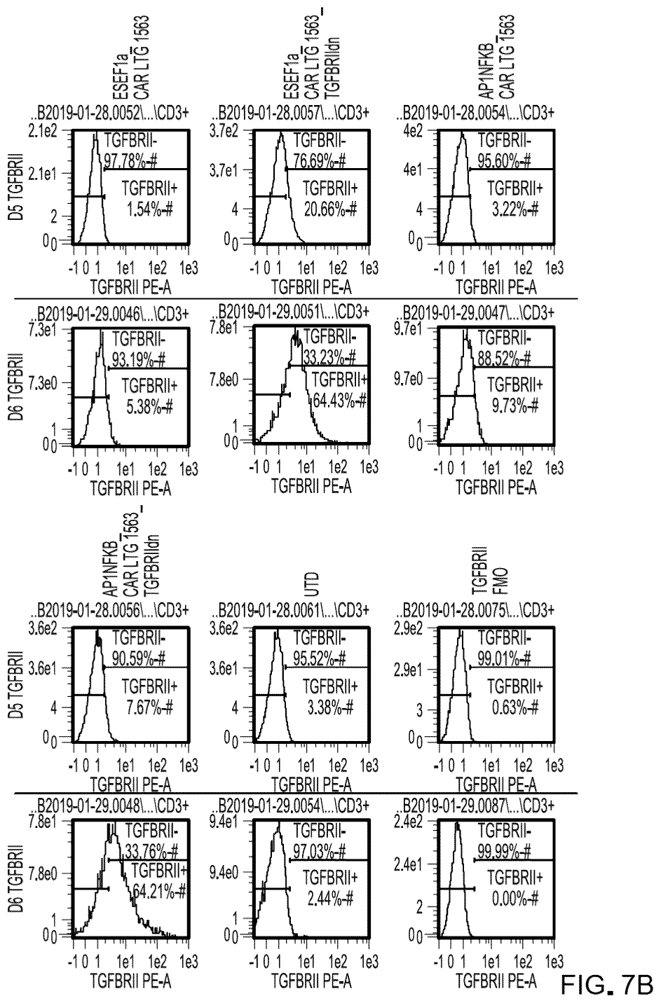

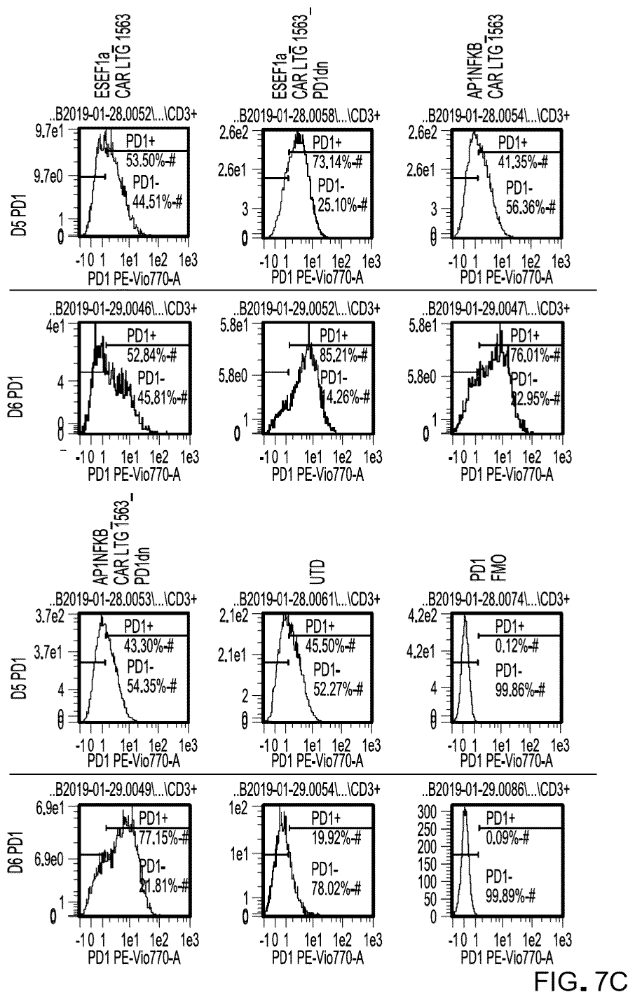

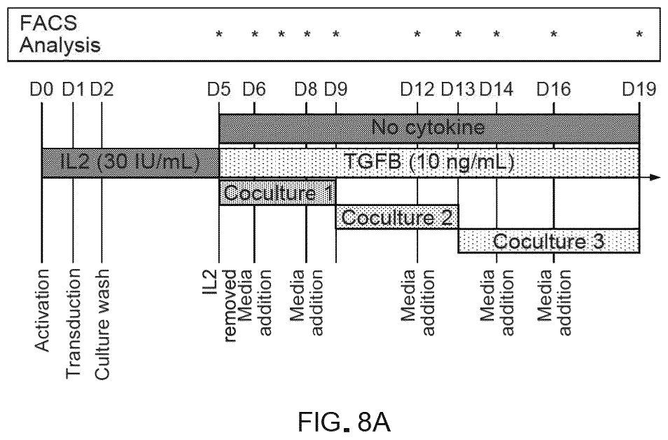

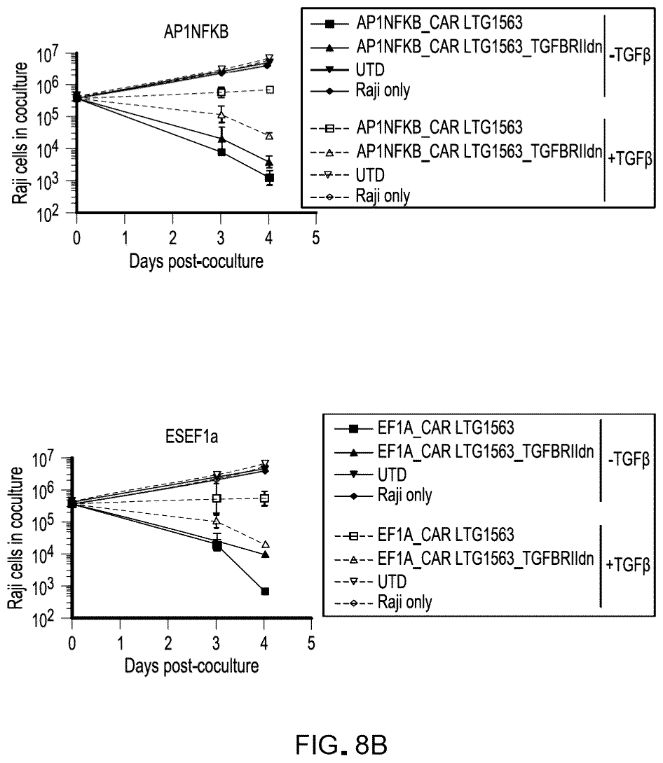

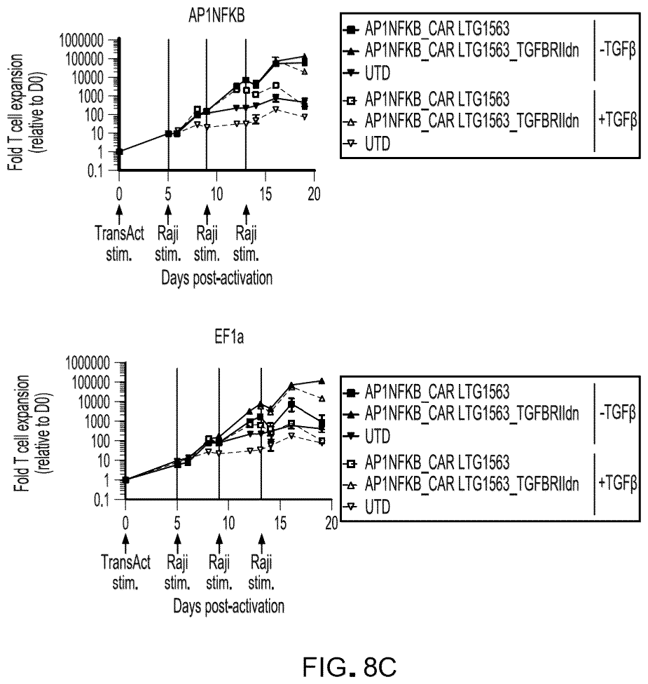

CAR T co-incubation with target cells was examined for a total of fifteen days (Coculture 1-3), during this time the numbers of viable Raji and T cells were enumerated by flow cytometric analysis (FIG. 8A). Whereas Raji cells in Raji only and UTD control groups proceeded to grow unhindered, CAR LTG1563 T cells strongly suppressed Raji expansion with all vectors in the absence of TGFβ, while the AP1NFKB-CAR LTG1563 and EF1a-CAR LTG1563 vectors displayed poor cytotoxicity of Raji cells in the presence of TGFβ. Strikingly, in the vector EF1a-CAR LTG1563-TGFBRIIdn, the dominant negative receptor TGFBRIIdn restored CAR-dependent cytotoxicity to a great extent in the presence of TGFβ (FIG. 8B). Similar restoration of CAR-dependent cytotoxicity was also observed for the AP1NFKB-CAR LTG1563-TGFBRIIdn vector, despite initially lower TGFBRII expression in these vectors (FIG. 7B). This indicates that the inducible nature of the AP1-NFKB promoter can be used to successfully express additional proteins beyond CARs, in a manner regulated by the antigen signaling through the CAR. The restoration of cytotoxicity in the presence of TGFβ for the EF1a-CAR LTG1563-TGFBRIIdn and AP1NFKB-CAR LTG1563-TGFBRIIdn vectors was also associated with a marked increase in CAR LTG1563-dependent T cell expansion throughout Cocultures 1-3 (FIG. 8C). Overall, this demonstrates successful use and induction of CARs with auxiliary components as a single-vector system using the inducible AP1-NFKB promoter.

### Example 3

Characterization of CAR19 LTG1563 with TGFBRIIdn and PD-1dn Decoy Components in the Presence of TGFβ or PD-L1 Ligands

### Materials and Methods

### Cell Lines Used to Demonstrate CAR Activity

The Burkitt lymphoma cell line Raji cell line, the chronic myelogenous leukemia line K562 line and reagents were purchased from American Tissue Culture Collection (ATCC, Manassass, VA), unless otherwise noted. Cells were cultured in RPMI-1640 medium supplemented with 10% heat-inactivated fetal bovine serum (FBS, Hyclone, Logan, UT) and 2 mM L-Glutamax (Thermo Fisher Scientific, Grand Island, NY). Human Embryonic kidney line 293T was purchased from ATCC and propagated in CD FortiCho medium (Gibco/Thermo Fisher Scientific, Grand Island, NY). Single-cell clones of luciferase-expressing cell lines were generated by stably transducing wild-type tumor lines with lentiviral vector encoding firefly luciferase (Lentigen Technology, Inc., Gaithersburg, MD), followed by cloning and selection of luciferase-positive clones. GFP- and PDL1-expressing cell lines by stably transducing luciferase-enabled tumor lines with lentiviral vector encoding GFP and/or PD-L1 (PDL1: AA 1-290, Uniprot ID: Q9NZQ7), followed by magnetic or FACS-based separation of GFP+ and PD-L1+ cells.

### Primary Human T Cells Used to Demonstrate CAR Activity

Whole blood was collected from healthy volunteers at Oklahoma Blood Institute (OBI) with donors' written consent. Processed buffy coats were purchased from OBI (Oklahoma City, OK). The CD4-positive and CD8-positive human T cells were purified from buffy coats via positive selection using a 1:1 mixture of CD4- and CD8-MicroBeads (Miltenyi Biotec, Bergisch Gladbach, Germany) according to manufacturer's protocol.

### Primary T Cell Transduction

Human primary CD4+ and CD8+ T cells from normal donors were cultivated in TexMACS medium at a density of 1×106 cells/ml, activated with CD3/CD28 MACS® GMP T Cell TransAct reagent on day 0 (all reagents from Miltenyi Biotec), and transduced on day 1 with LV encoding CAR constructs overnight, and media exchanged on day 3. Supplementation with cytokines (IL-2, TGFβ; Miltenyi Biotec, Bergisch Gladbach, Germany) was performed as described. Cultures were propagated until harvest on day 8 for co-incubation analysis.

### Immune Effector Assays (CTL and Cytokine)

For long-term co-incubation assays, CAR T effector and GFP-expressing target cells were propagated as above, and flow cytometric analysis was utilized to determine the extent of target cell population killing, and CAR T population survival and expansion. Cells were gated based on forward and side scatter, and viable (7AAD-negative) cells. Percentages of surviving cells post in co-cultures were determined based on GFP positivity for Raji targets and CD3 for CAR T effectors. In addition, CAR-T expression in live CD3 positive cells was determined by staining with CD19 Fc peptide, followed by anti-Fc (Fab′)2—FL reagent. PD1 and TGFBRII expression was also determined by flow cytometry.

### Results

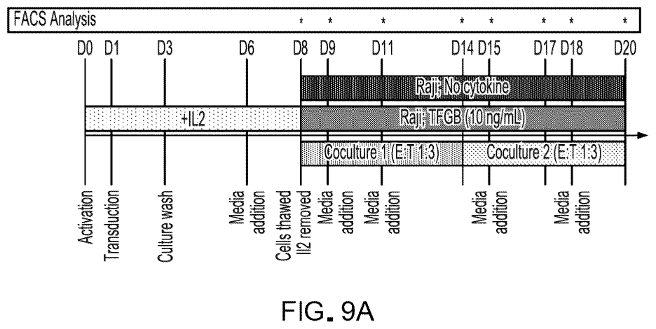

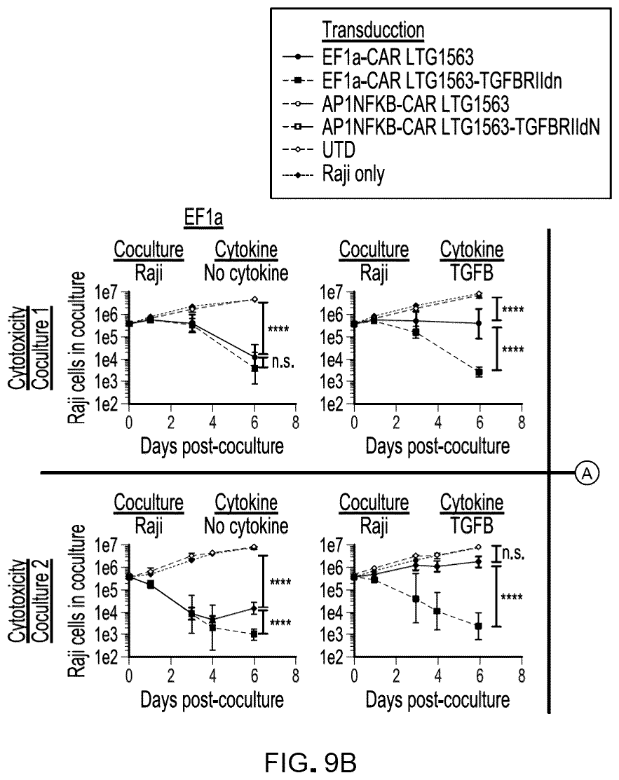

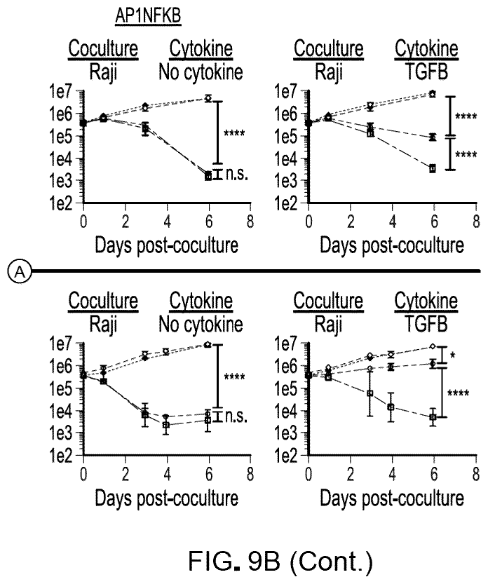

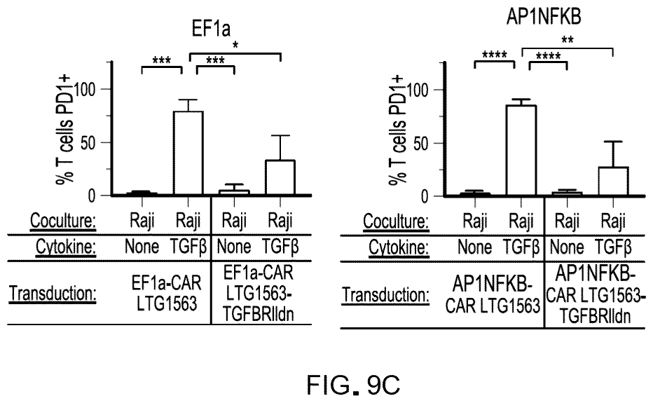

Prior experiments (FIG. 8) with CAR LTG1563 constructs expressing the auxiliary component TGFBRIIdn demonstrated significant protection from the anti-inflammatory effects of the immunosuppressive cytokine TGFβ. Therefore, these results were extended to include additional T cell donors and analysis of additional parameters of CAR T efficacy (cytokine production, activation marker expression, and exhaustion marker expression). Briefly, T cells were purified from blood of three unrelated healthy donors by immunogenic selection using a 1:1 mixture of CD4 and CD8 beads (Miltenyi Biotec). Cells were transduced with CAR LTG1563 constructs with auxiliary components in the presence of IL2 supplementation, as described in the materials and methods. The experimental groups included CAR LTG1563 or CAR LTG1563-TGFBRIIdn driven by the EF1α promoter, or with AP1/NFκB_RE-driven CAR LTG1563, CAR LTG1563-TGFBRIIdn regulated self-driving CARs with auxiliary components at 0.5% vol/vol LV preparation (FIG. 9A). Untransduced cells (UTD) cultured under same conditions were used as a negative control. The expression of CAR LTG1563 and the auxiliary components (TGFBRIIdn) was assessed by flow cytometric enumeration both prior to and after CD19 antigen stimulation with Raji NHL tumor cells, which displayed similar characteristics to prior experiments (FIG. 8 and data not shown).

Further, in this experiment, the CAR LTG1563-dependent cytotoxicity was assessed by coculture of CAR T cells with CD19+ Raji NHL cells stably expressing GFP, in the presence or absence of the immunosuppressive cytokine TGFβ (10 ng/mL). A very low effector to target ratio was used (1:3 CAR T:Raji cells), and CD19-dependent cytotoxicity was assessed by flow cytometric enumeration of the GFP+ Raji cells from D8-D14 post-activation (Coculture 1, FIG. 9A). Long-term function and expansion of the CAR T cells was assessed by a secondary re-stimulation of the cells at a similar effector to target ratio (1:3 Coculture 1:Raji cells) from D14-D20 post-activation (Coculture 2). CAR T co-incubation with target cells was examined for a total of fourteen days (Coculture 1-2), during this time the numbers of viable Raji and T cells were enumerated by flow cytometric analysis (FIG. 9B). Whereas Raji cells in Raji only and UTD control groups proceeded to grow unhindered, CAR LTG1563 T cells strongly suppressed Raji expansion with all vectors in the absence of TGFβ, while the AP1NFKB-CAR LTG1563 and EF1a-CAR LTG1563 transduced T cells displayed poor cytotoxicity of Raji cells in the presence of TGFβ. Strikingly, in the vector EF1α-CAR LTG1563-TGFBRIIdn, the dominant negative receptor TGFBRIIdn restored CAR-dependent cytotoxicity to a great extent in the presence of TGFβ (FIG. 8B). Similar restoration of CAR-dependent cytotoxicity was also observed for the AP1NFKB-CAR LTG1563-TGFBRIIdn vector, despite initially lower TGFBRII expression in these vectors (data not shown). These results extend prior findings that the inducible nature of the AP1-NFKB promoter can be used to successfully express additional proteins beyond CARs, in a manner regulated by the antigen signaling through the CAR.

Additional T cell parameters were assessed in the course of the experiment. In the presence of TGFβ, the expression of the auxiliary component TGFBRIIdn restored high-level production of pro-inflammatory cytokines such as IL2 and expression of activation markers such as CD25 (data not shown). TGFβ supplementation of CAR T cocultures lead to markedly high expression levels of the exhaustion marker PD1 (FIG. 9C), as measured at the end of Coculture 2 (D20 post-activation). Of note, a significant decrease was observed in expression of the exhaustion marker PD1 in both AP1NFKB-CAR LTG1563-TGFBRIIdn and EF1a-CAR LTG1563-TGFBRIIdn-expressing CAR T cells in the presence of TGFβ relative to APlNFKB-CAR LTG1563 and EF1a-CAR LTG1563-expressing CAR T cells under the same conditions (FIG. 9C). The reduced TGFβ-dependent expression of PD1 lead to the hypothesis that TGFBRIIdn expression could protect CAR-T cells not only from the anti-inflammatory effects of TGFβ, but also from the negative effects of PD-1 signaling.

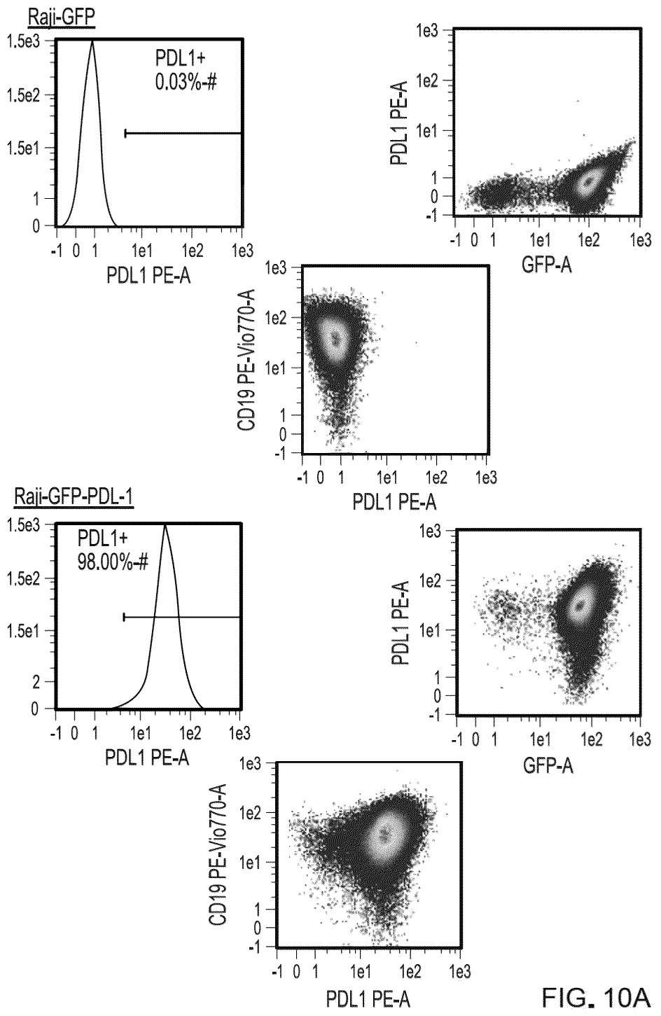

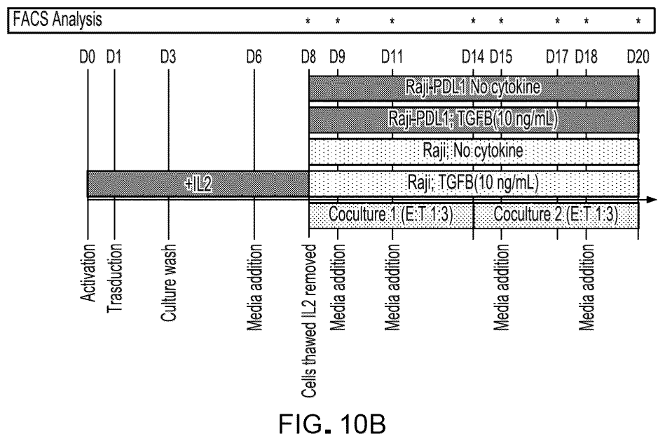

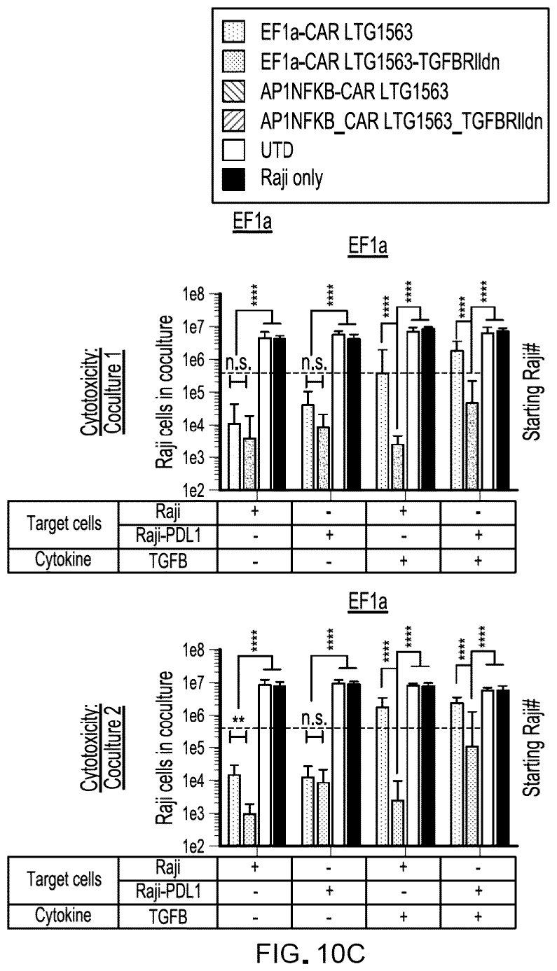

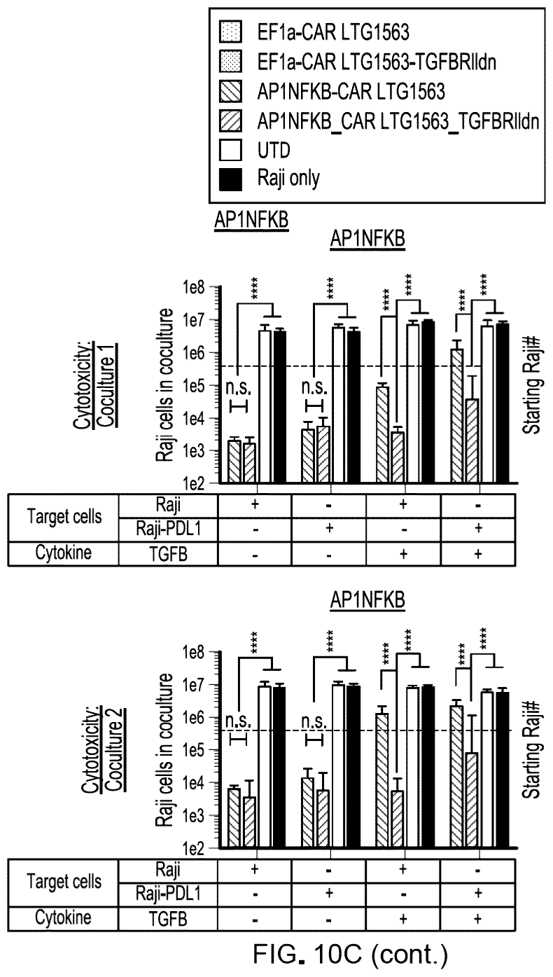

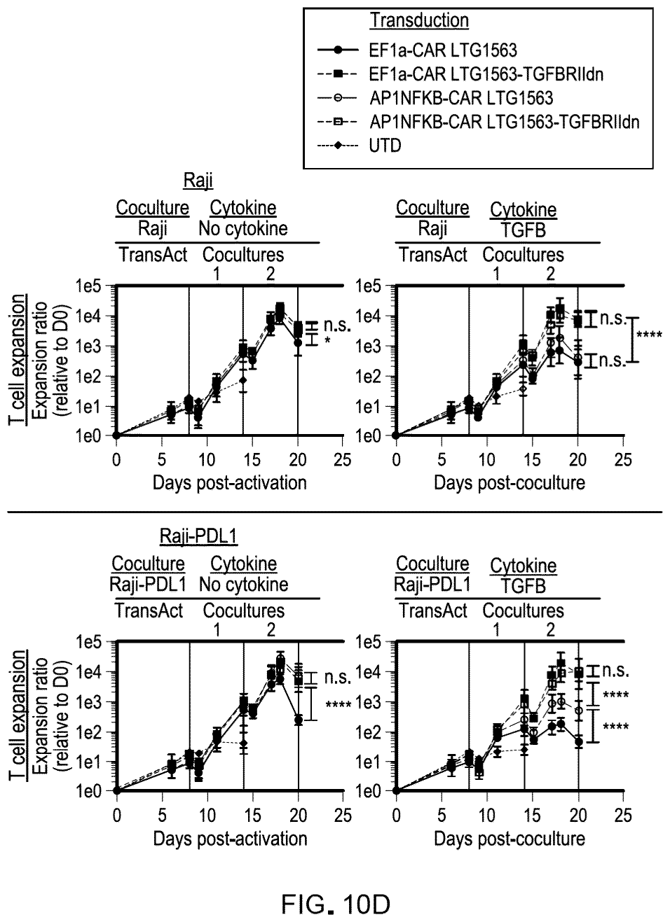

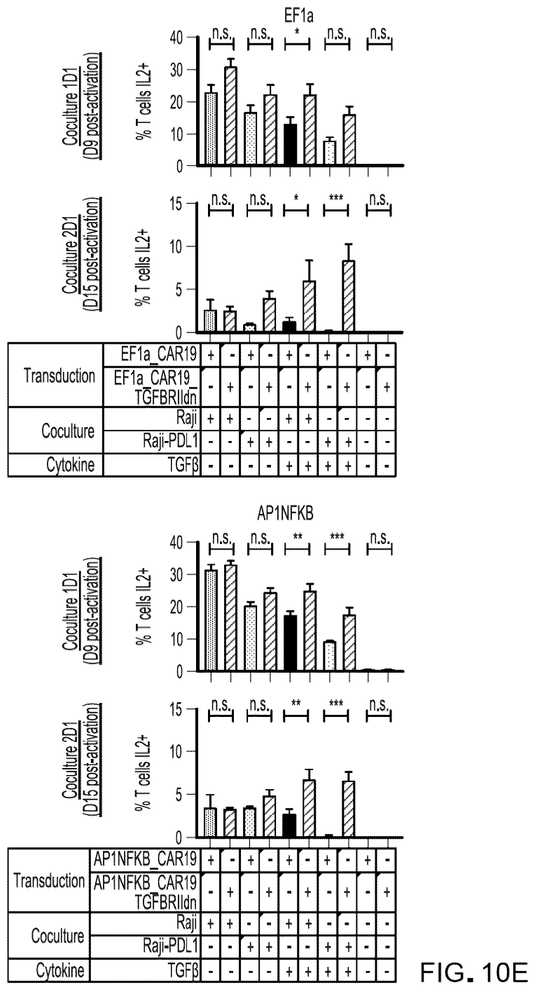

Consequently, the ability of the auxiliary CAR component TGFBRIIdn to protect from the immunosuppressive effects of PD-1/PD-L1 signaling was assessed by transducing CD19+ target Raji-GFP NHL cells with a lentivector stably expressing PD-L1 to create the cell line Raji-GFP-PDL1. Magnetic selection of PDL1 cells was performed, and stable expression of PDL1 over a period of weeks on Raji-GFP-PDL1 cells was assessed by flow cytometry (data not shown). As previously, T cells were purified from blood of three unrelated healthy donors by immunogenic selection using a 1:1 mixture of CD4 and CD8 beads (Miltenyi Biotec). Cells were transduced with CAR LTG1563 constructs with auxiliary components in the presence of IL2 supplementation, as described in the materials and methods. The experimental groups included CAR LTG1563 or CAR LTG1563-TGFBRIIdn driven by the EF1α promoter, or with AP1/NFκB_RE-driven CAR LTG1563, CAR LTG1563-TGFBRIIdn regulated self-driving CARs with auxiliary components at 0.5% vol/vol LV preparation (FIG. 9A). Untransduced cells (UTD) cultured under same conditions were used as a negative control. CAR LTG1563-dependent cytotoxicity was assessed by coculture of CAR T cells with CD19+ Raji-GFP or CD19+ Raji-GFP-PDL1 NHL cells, in the presence or absence of the immunosuppressive cytokine TGFβ (10 ng/mL). A very low effector to target ratio was used (1:3 CAR T:Raji cells), and CD19-dependent cytotoxicity was assessed by flow cytometric enumeration of the GFP+ Raji cells from D8-D14 post-activation (Coculture 1, FIG. 10C). Long-term function and expansion of the CAR T cells was assessed by a secondary re-stimulation of the cells at a similar effector to target ratio (1:3 Coculture 1:Raji cells) from D14-D20 post-activation (Coculture 2). CAR T co-incubation with target cells was examined for a total of fourteen days (Coculture 1-2), during this time the numbers of viable Raji and T cells were enumerated by flow cytometric analysis (FIG. 10C).

While PD-L1 expression alone on target Raji NHL cells was not sufficient to significantly affect CAR LTG1563-dependent cytotoxicity (FIG. 10A), a synergistic decrease was observed in the cytotoxic ability of AP1NFKB-CAR LTG1563 and EF1a-CAR LTG1563-expressing CAR T cells in the presence of both target PD-L1 expression and TGFβ supplementation. Notably, the co-expression of the auxiliary component TGFBRIIdn was able to restore the CD19-dependent killing capacity of CAR T cells in the presence of both target PD-L1 expression and TGFβ supplementation, although not to the degree observed in the absence of both PD-1/PD-L1 and TGFβ signaling. This is in agreement with the partial, but not complete, prevention of TGFβ-induced PD-1 expression observed in the presence of TGFBRIIdn expression (FIG. 9C).

The effects of the TGFBRIIdn transgene on CAR LTG1563-dependent T cell expansion were also assessed in the course of this experiment. Prior results were recapitulated (FIG. 5C) demonstrating that AP1NFKB-CAR LTG1563 demonstrated superior CD19 antigen-dependent T cell expansion relative to EF1a-CAR LTG1563 (FIG. 9C, 1st panel from left). Notably, the greater CAR19-dependent T cell expansion previously observed with AP1-NFKB_CAR19 relative to EF1α_CAR19 became more pronounced when Raji-PDL1 targets were used in coculture, even in the absence of TGFβ (FIG. 9C, 3rd panel from left). It is possible that any negative effects of tonic CAR signaling, as would potentially be the case during constitutive CAR expression (FIG. 4), become more apparent under conditions where PD1 checkpoint signaling occurs, which would be the case in coculture with Raji-PDL1 cells. In all circumstances, in the presence of both TGFβ supplementation and target cell PDL1 expression, the expression of the TGFβRIIdn transgene significantly increased the CAR19-dependent T cell expansion (FIG. 9C, 4th panel from the left), indicating protection from anti-inflammatory PD1/PD-L1 signaling by reducing TGFβ signaling.

### REFERENCE TO THE SEQUENCE LISTING

This application contains a Sequence Listing electronically submitted in ASCII format created on Mar. 5, 2020, entitled 42449_0042WO1 SL.txt, and is 384,255 bytes in size. The Sequence Listing is incorporated by reference.

1. An isolated nucleic acid molecule encoding a therapeutic payload operably connected to a surface antigen-regulated inducible promoter comprising a nucleotide sequence comprising SEQ ID NO: 138, or a sequence with 85%, 90%, 95%, 96%, 97%, 98%, or 99% identity thereof, and wherein the therapeutic payload comprises a chimeric antigen receptor (CAR) comprising at least one extracellular antigen binding domain comprising mesothelin, CD33, CD19, CD19/CD20, CD22, CD19/CD22, ROR1, CD123, or CD38 antigen binding domain, at least one linker domain, at least one transmembrane domain, and at least one intracellular signaling domain, and wherein the surface antigen-regulated inducible promoter adjusts its level of transcription dependent upon the level of expression of surface antigen on the target cell, thereby achieving a T-cell response precisely regulated to the level of target antigen present in the tumor milieu.

2.-14. (canceled)

15. A chimeric antigen receptor (CAR) encoded by the isolated nucleic acid molecule of claim 1.

16.-23. (canceled)

24. A vector comprising a nucleic acid molecule of claim 1.

25. The vector of claim 24, wherein the vector is selected from the group consisting of a DNA vector, an RNA vector, a plasmid vector, a cosmid vector, a herpes virus vector, a measles virus vector, a lentivirus vector, an adenoviral vector, and a retrovirus vector, or a combination thereof.

26. An isolated cell comprising the vector of claim 24.

27. The isolated cell of claim 26, wherein the isolated cell is a T cell.

28. The isolated cell of claim 26, wherein the T cell is a CD8+ T cell.

29. The isolated cell of claim 26, wherein the isolated cell is a human cell.

30. A method of making a cell comprising transducing a T cell with a vector of claim 24.

31. A method of generating a population of RNA-engineered cells comprising introducing an in vitro transcribed RNA or synthetic RNA into a cell, where the RNA comprises a nucleic acid molecule of claim 1.

32.-33. (canceled)

34. A pharmaceutical composition comprising an anti-tumor effective amount of a population of human T cells, wherein the population of human T cells comprise a therapeutic payload operably connected to a surface antigen-regulated inducible promoter encoded by a nucleic acid sequence comprising SEQ ID NO: 138, or a combination thereof, wherein said surface antigen-regulated inducible promoter adjusts its level of transcription dependent upon the level of expression of surface antigen on the target cell thereby achieving a T-cell response precisely regulated to the level of target antigen present in the tumor milieu, wherein said therapeutic payload comprises a nucleic acid sequence that encodes a chimeric antigen receptor (CAR), and wherein said CAR comprises at least one extracellular antigen binding domain comprising mesothelin, CD33, CD19, CD19/CD20, CD22, CD19/CD22, ROR1, CD123, or CD38 antigen binding domain or a combination thereof, at least one linker domain, at least one transmembrane domain, at least one intracellular signaling domain, and wherein the population of human T cells are T cells of a human having a cancer.

35. (canceled)

36. The pharmaceutical composition of claim 34, wherein the T cells are T cells of a human having a hematological cancer.

37. The pharmaceutical composition of claim 36, wherein the hematological cancer is leukemia or lymphoma.

38. The pharmaceutical composition of claim 37, wherein the leukemia is acute myeloid leukemia (AML), blastic plasmacytoid dendritic cell neoplasm (BPDCN), chronic myelogenous leukemia (CML), chronic lymphocytic leukemia (CLL), acute lymphoblastic T cell leukemia (T-ALL), or acute lymphoblastic B cell leukemia (B-ALL).

39. The pharmaceutical composition of claim 37, wherein the lymphoma is mantle cell lymphoma, non-Hodgkin's lymphoma or Hodgkin's lymphoma.

40. The pharmaceutical composition of claim 36, wherein the hematological cancer is multiple myeloma.

41.-50. (canceled)


SEQUENCE LISTING (189 sequences)

SEQ ID NO: 1-3 - skipped (3 entries)

SEQ ID NO: 4 - DNA, 10 bp, synthetic construct
  source 1..10: mol_type = other DNA
gggaatttcc

SEQ ID NO: 5 - DNA, 10 bp, synthetic construct
  source 1..10: mol_type = other DNA
gggactttcc

SEQ ID NO: 6 - skipped

SEQ ID NO: 7 - DNA, 1494 bp, synthetic construct
  source 1..1494: mol_type = other DNA
atgctgctgctggtgaccagcctgctgctgtgcgaactgccgcatccggcgtttctgctgattccggaagtgcaattggtgcagagcggtggaggacttgtgaaacctggtggatccctgagactttcctgtgccgcttcgggcttcaccttctcggactactacatgtcctggattcgccaggcccctggaaagggactggaatgggtgtcatacatcagctcctccggttccaccatctactatgccgattccgtgaagggcagattcaccatctcgcgcgacaacgccaagaacactctctatctgcaaatgaactcactgcgggctgaggacaccgcggtctactactgcgcccgggacctcagcggaaagtccagcggatggtcccattacttcgattactggggacagggaaccctggtcaccgtgtccagcggcggggggggctcgggtggcggcggctccggcggcggcgggagcaacttcatgctgactcagccccactccgtgtccgagagcccgggaaagaccgtgactatttcgtgcacacggtcctccgggagcattgcgaacaactacgtgcagtggtaccagcagcggcccgatagggccccaaccactgtgatctacgaagatgaccagcggccgtctggagtcccggaccgcttctcggggtccatcgactcatcatccaattccgcatcgctgacgatcagcggactgaagatcgaggacgaagccgattactactgccagtcctacgacggcaccaactgggtctttgggggtggaaccaagctgactgtgctcggagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 8 - protein, 498 aa, synthetic construct
MLLLVTSLLLCELPHPAFLLIPEVQLVQSGGGLVKPGGSLRLSCAASGFTFSDYYMSWIRQAPGKGLEWVSYISSSGSTIYYADSVKGRFTISRDNAKNTLYLQMNSLRAEDTAVYYCARDLSGKSSGWSHYFDYWGQGTLVTVSSGGGGSGGGGSGGGGSNFMLTQPHSVSESPGKTVTISCTRSSGSIANNYVQWYQQRPDRAPTTVIYEDDQRPSGVPDRFSGSIDSSSNSASLTISGLKIEDEADYYCQSYDGTNWVFGGGTKLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 1473 bp, synthetic construct
  source 1..1473: mol_type = other DNA
atgctgctgctggtgaccagcctgctgctgtgcgaactgccgcatccggcgtttctgctgattccggaagtgcaacttgtcgaaagcggtggaggtcttgtccaacctggtcgctccctgaggctctcgtgtgccgcgagcggattcaccttctcatcgtacgctatgtcctgggtcagacaggctcctggaaagggcctggaatgggtggccgtgatctcctacgacggcagcaacaagtattacgccgactcagtgaagggccggttcaccatttcccgggacaacagcaagaacaccctgtacttgcaaatgaactccctgcgggccgaggataccgcggtgtactactgcgcccacctccgctttggatacggaatggatgtctggggacagggaactaccgtgaccgtgtcgtccgggggggggggaagcggcggcgggggatcgggtggcggcggatcccagactgtggtcacccaagagccttcactgaccgtgtccccgggtggcaccgtgacgctgacttgcgcgtcatctaccggggccgtgacctcggaccactacccctgctggttccagcagaaacccggacatccaccgagagccctggtgtactccactgacaccatccactcctggactccggcccggttctccggaagcctcctgggcgggaaggccgcactgacagtgtccggagtgcagcccgaggatgaagccgactactactgtctgctctactatgggggagcacgcgtgttcggtggcggcactcagctgaccgtgctgggagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 10 - protein, 491 aa, synthetic construct
MLLLVTSLLLCELPHPAFLLIPEVQLVESGGGLVQPGRSLRLSCAASGFTFSSYAMSWVRQAPGKGLEWVAVISYDGSNKYYADSVKGRFTISRDNSKNTLYLQMNSLRAEDTAVYYCAHLRFGYGMDVWGQGTTVTVSSGGGGSGGGGSGGGGSQTVVTQEPSLTVSPGGTVTLTCASSTGAVTSDHYPCWFQQKPGHPPRALVYSTDTIHSWTPARFSGSLLGGKAALTVSGVQPEDEADYYCLLYYGGARVFGGGTQLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1467 bp, synthetic construct
  source 1..1467: mol_type = other DNA
atgctgctgctggtgaccagcctgctgctgtgcgaactgccgcatccggcgtttctgctgattccgcaagtccaactcgtccagtccggtgccgaagtcaagaagcctggctcatccgtgaaagtgtcctgcaaagcatcgggcggaaccttctcctcctatgccatttcctgggtccgccaggcaccgggccagggtctggagtggatgggcgggattatccctatcttcggaactgccaaccacgcccaaaagttccagggacgcgtgaccattaccgccgatgaatcaacctcaaccgcctacatggaactgtccagcttgaggtccgaggacaccgccgtgtactactgcgcgttcatgatggtgccggagtactactttgactactggggccagggaacccttgtgaccgtgtcgtccggtggtggcggatccgggggggggggatctgggggcggcggaagcgatatccagatgacccagtcgccatcgagcctgtccgcttccgtgggcgacagagtgacgatcacttgccgggcttcacaaggcatcagaaatgacctgggctggtatcagcagaagcccggagaagcgcccaagcggctgatctacgcggccagcaccctgcaaaacggagtgccttcgcggttctccgggagcggctccggaactgacttcactctgactattaacagcctccagcccgaggatttcgccacatactactgtcagcagtacaacagctacccgtacaccttcggacagggaactaagctcgaaatcaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 12 - protein, 489 aa, synthetic construct
MLLLVTSLLLCELPHPAFLLIPQVQLVQSGAEVKKPGSSVKVSCKASGGTFSSYAISWVRQAPGQGLEWMGGIIPIFGTANHAQKFQGRVTITADESTSTAYMELSSLRSEDTAVYYCAFMMVPEYYFDYWGQGTLVTVSSGGGGSGGGGSGGGGSDIQMTQSPSSLSASVGDRVTITCRASQGIRNDLGWYQQKPGEAPKRLIYAASTLQNGVPSRFSGSGSGTDFTLTINSLQPEDFATYYCQQYNSYPYTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 66 bp, synthetic construct
  source 1..66: mol_type = other DNA
atgctgctgctggtgaccagcctgctgctgtgcgaactgccgcatccggcgtttctgctgattccg

SEQ ID NO: 14 - protein, 22 aa, synthetic construct
MLLLVTSLLLCELPHPAFLLIP

SEQ ID NO: 15 - DNA, 1500 bp, synthetic construct
  source 1..1500: mol_type = other DNA
atgctgctgctggtgaccagcctgctgctgtgcgaactgccgcatccggcgtttctgctgattccgcaactccaactccaagaatctggaccaggcctcgtgaagccctcccaaactctgtccctgacctgtaccgtgtcgggtggaagcatttcgagcggtggatactactggtcctggatcaggcagcatcctggaaagggactggagtggattgggtacatctactactccggctcaacctactacaacccgtccttgaaatcgcgcgtgacgatctccgtggacacttcaaagaaccagttcagcctgaagctttcctccgtgaccgcggccgatacagcggtgtactactgcgctcgggatcagagcgtggccgaccctggtggcggctactactactacggaatggatgtctggggacagggaaccaccgtgactgtgtccagcgggggcggcggatccgggggggggggatcgggcggcggcggttcgcagtccgtgctgacccagccacctagcgtgtcagtggcaccgggacagaccgcctccatttcctgcgggggaaatgacttcggtagccgctccgtgtcatggtatcaccagaagccgggacaggccccggtgctggtcatctatgacgacaacgacagaccctcgggcatccccgaacggttttcgggaagcacctccggagacactgccaccctgaccatctcccgggtcgaggtcggcgatgaagccgattactactgccaagtctgggacgacgactccgaccactgggtgttcggcggcggaactaagctgactgtgctgggggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 16 - protein, 500 aa, synthetic construct
MLLLVTSLLLCELPHPAFLLIPQLQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDQSVADPGGGYYYYGMDVWGQGTTVTVSSGGGGSGGGGSGGGGSQSVLTQPPSVSVAPGQTASISCGGNDFGSRSVSWYHQKPGQAPVLVIYDDNDRPSGIPERFSGSTSGDTATLTISRVEVGDEADYYCQVWDDDSDHWVFGGGTKLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 1467 bp, synthetic construct
  source 1..1467: mol_type = other DNA
atgctgctgctggtgaccagcctgctgctgtgcgaactgccgcatccggcgtttctgctgattccgcaagttcagctgcaagaatcaggacctgggcttgtcaaaccatctgaaaccctcagcttgacttgtaccgtatcaggagggtcaatttcaagctcatcctactattggggatggatcagacaaccacccgggaaagggctcgagtggatagggtccatatattacagcggatctacatactacaacccgtcattgaagtccagggtaacgattccggtggacactagcaagaatcagtttagcctcaagttgagcagtgtaactgctgcggacacggcggtatattattgtgctcgacacctcggtggagatgcttttgacatatggggtcaagggacaacagtcaccgttagctcaggtggagggggtagcggggggggcggatctgggggaggcggttcattgcccgtacttacacagccaccctctgtcagcgtcgcacctggacaaaccgctcgcatcacctgtggcggaaataatataggttccaagtctgttcattggtatcagcagaaaccgggacaggcccccgtccttgtggtgtatgatgattctgataggccatctggtatcccagaacggttttcaggtagcaattcagggaatactgccactctcactattagcggtactcaagctatggatgaggccgactatttttgccagagctacgactctagtaacccagtcgtgttcgggggagggacccagttgaccgtgctggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 18 - protein, 489 aa, synthetic construct
MLLLVTSLLLCELPHPAFLLIPQVQLQESGPGLVKPSETLSLTCTVSGGSISSSSYYWGWIRQPPGKGLEWIGSIYYSGSTYYNPSLKSRVTIPVDTSKNQFSLKLSSVTAADTAVYYCARHLGGDAFDIWGQGTTVTVSSGGGGSGGGGSGGGGSLPVLTQPPSVSVAPGQTARITCGGNNIGSKSVHWYQQKPGQAPVLVVYDDSDRPSGIPERFSGSNSGNTATLTISGTQAMDEADYFCQSYDSSNPVVFGGGTQLTVLAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 1476 bp, synthetic construct
  source 1..1476: mol_type = other DNA
atgctgctgctggtgaccagcctgctgctgtgcgaactgccgcatccggcgtttctgctgattccgcaggcggcccaggtacagctgcagcagtcaggggctgaggtgaagaagcctgggtcctcggtgaaggtctcctgcaaggcttctggaggcaccttcagcagctatgctatcagctgggtgcgacaggcccctggacaagggcttgagtggatgggatggatcaaccctaacagtggtggcacaaactatgcacagaggtttcagggcagggtcaccatgaccagggacacgtccatcagcacagcctacatggagctgagcaggctgagatctgacgacacggccgtgtattactgtgcgagttataatgatgcttttgatatctggggccaaggcaccctggtcaccgtctcctcaggaggtggcgggtctggtggtggcggtagcggtggtggcggatccaattttatgctgactcagccccactctgtgtcggagtctccggggaagacggtaaccatctcctgcacccgcagcagtggcagcattgccagcaactatgtgcagtggtaccagcagcgcccgggcagtgcccccaccattgtgatctatgaggatgatcaaagaccctctggggtccctgatcggttctctggctccatcgacacctcctccaactctgcctccctcaccatctctggactgcagagtgaggacgaggctgactactactgtcagtcttatgagcccggcaatggggtattcggcggagggaccaaggtcaccgtcctagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 20 - protein, 492 aa, synthetic construct
MLLLVTSLLLCELPHPAFLLIPQAAQVQLQQSGAEVKKPGSSVKVSCKASGGTFSSYAISWVRQAPGQGLEWMGWINPNSGGTNYAQRFQGRVTMTRDTSISTAYMELSRLRSDDTAVYYCASYNDAFDIWGQGTLVTVSSGGGGSGGGGSGGGGSNFMLTQPHSVSESPGKTVTISCTRSSGSIASNYVQWYQQRPGSAPTIVIYEDDQRPSGVPDRFSGSIDTSSNSASLTISGLQSEDEADYYCQSYEPGNGVFGGGTKVTVLAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 1488 bp, synthetic construct
  source 1..1488: mol_type = other DNA
atgctgctgctggtgaccagcctgctgctgtgcgaactgccgcatccggcgtttctgctgattccgcaagaacagcttgtagagtccggcggtagattggtgacaccgggggggagccttaccctgtcttgtaaggcatctgggttcgatttcagtgcgtattatatgagctgggttcggcaggcgcccgggaaggggctggaatggatagccactatatacccgtcatccggcaagacttactacgcgacttgggtaaacgggaggtttacgataagctcagataacgcccaaaacacggttgatctccaaatgaatagcttgaccgccgctgatagggcgacctatttctgtgcgcgggactcttacgctgatgacggggccctcttcaatatatggggaccgggaacgctcgtaaccatatcatctggaggaggtgggagcggaggcggagggtcaggtgggggcgggagcgaactcgtacttacacaatctccaagcgtaagcgcagcgttggggagtccagcaaagatcacctgcactttgtcaagcgcccacaaaacggatacgatagattggtatcagcaactccaaggtgaagcgccacgatatctcatgcaggtacagagcgacgggagttatactaagaggcccggggtcccagacagattcagtggcagcagttcaggtgccgacagatacctgataataccctcagttcaagccgatgatgaagccgattactactgtggggctgactacataggtgggtatgttttcgggggcggcactcaattgacagttacaggggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 22 - protein, 496 aa, synthetic construct
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1464 bp, synthetic construct
  source 1..1464: mol_type = other DNA
atgctgctgctggtgaccagcctgctgctgtgcgaactgccgcatccggcgtttctgctgattccggaagtgcaactcgtcgaaactggagccgaagtgaaaaagcctggagcgtccgtcaaagtgtcgtgcaaggcctccggctacaccttcacgacctactacgtgcactgggtcagacaggctccgggtcaagggctggagtggatgggcatcattaacccctccggtggaagcacctcctatgcgcaaaagttccagggtcgcgtcaccatgactcgcgatacctccacttccactgtgtacatggaactgagctccctgaggtccgaggacaccgccgtgtactactgcgcacgggatggaggcttgggcggctacgaggcttggggacagggcaccctcgtgactgtgtcaagcggagggggtggatccggagggggaggatcaggcggtggtggaagcgatatccagcttacccagtcgccttccgcgctgtctgcatcggccggcgacagagtgacaattacctgtcaagccagccaggacatctccaactatctgaactggtaccagcagaagcccggaaaggctccgaagctgctgatctacgacgccagcaacctggaacggggcgtgccatcacggttctcgggatcagggtcgggcactgagttcaccttcaccatctcctccctccaacccgaggacattgccacctactactgccagcagtacgacaacctcccgatcacctttggacaggggactcgcctggaaatcaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 24 - protein, 488 aa, synthetic construct
MLLLVTSLLLCELPHPAFLLIPEVQLVETGAEVKKPGASVKVSCKASGYTFTTYYVHWVRQAPGQGLEWMGIINPSGGSTSYAQKFQGRVTMTRDTSTSTVYMELSSLRSEDTAVYYCARDGGLGGYEAWGQGTLVTVSSGGGGSGGGGSGGGGSDIQLTQSPSALSASAGDRVTITCQASQDISNYLNWYQQKPGKAPKLLIYDASNLERGVPSRFSGSGSGTEFTFTISSLQPEDIATYYCQQYDNLPITFGQGTRLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 1503 bp, synthetic construct
  source 1..1503: mol_type = other DNA
atgctgctgctggtgaccagcctgctgctgtgcgaactgccgcatccggcgtttctgctgattccggaagtgcaattggtccagagcggaggaggacttgtgaagccaggcggatccctgagattgtcatgcgccgcatcggggttcaccttttcctcctactccatgaactgggtcagacaggcgcccggaaagggacttgaatgggtgtcgtccatttcctcctcctcgtcctacatctactacgccgactccgtgaagggccgcttcaccatctcccgggacaacgccaagaacagcctgtatctccaaatgaactccctgcgggccgaagatactgctgtgtattactgcgctcgggacttcccgtacgactcatcgggctattactcggacgcgttcgatatctggggccagggaactatggtcaccgtcagctctggtggcggtggttccggagggggtggatccggtggcggaggatcagagattgtgctgacccagtccccgctgtcactgcccgtgactccgggagagcctgcctcgatctcgtgtcggtccagccagtccctgctgcactcgaatgggtacaactacctcgattggtacctccaaaagcctgggcagtcaccccaactgctgatctacctcgggagcaacagagccagcggagtgcctgaccgctttagcggttccggatccggcaccgacttcaccctgaaaatcagccgggtggaagccgaggatgtcggcgtgtactactgcatgcaggcactgcagactctggggtacaccttcggccagggcacgaagctcgagatcaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 26 - protein, 501 aa, synthetic construct
MLLLVTSLLLCELPHPAFLLIPEVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDFPYDSSGYYSDAFDIWGQGTMVTVSSGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTLGYTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 28 - protein, 22 aa, synthetic construct
IWAPLAGTCGVLLLSLVITLYC

SEQ ID NO: 29 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 30 - protein, 47 aa, synthetic construct
TTTPAPRPPTPAPTIASQPLSLRPEACRPAAGGAVHTRGLDFACDIY

SEQ ID NO: 31 - protein, 42 aa, Homo sapiens
KRGRKKLLYIFKQPFMRPVQTTQEEDGCSCRFPEEEEGGCEL

SEQ ID NO: 32 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 34 - protein, 42 aa, synthetic construct; identical to SEQ ID NO: 31

SEQ ID NO: 35 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - DNA, 726 bp, synthetic construct
  source 1..726: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 38 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 39 - DNA, 66 bp, synthetic construct; identical to SEQ ID NO: 13
  source 1..66: mol_type = other DNA

SEQ ID NO: 40 - protein, 22 aa, synthetic construct
MLLLVTSLLLCELPHPAFLLIP

SEQ ID NO: 41 - DNA, 85 bp, synthetic construct
  source 1..85: mol_type = other DNA
ggctctgaaagtgctgttggaacaagaaaagaccttcttcaccttgctcgtgttgctggggtacctgtcctgcaaagtcacctgt

SEQ ID NO: 42 - protein, 29 aa, synthetic construct
MALKVLLEQEKTFFTLLVLLGYLSCKVTC

SEQ ID NO: 43 - DNA, 63 bp, synthetic construct
  source 1..63: mol_type = other DNA
atggcgctgccggtgaccgcgctgctgctgccgctggcgctgctgctgcatgcggcgcgcccg

SEQ ID NO: 44 - protein, 21 aa, synthetic construct
MALPVTALLLPLALLLHAARP

SEQ ID NO: 45 - DNA, 123 bp, synthetic construct
  source 1..123: mol_type = other DNA
cggtcgaagaggtccagactcttgcactccgactacatgaacatgactcctagaaggcccggacccactagaaagcactaccagccgtacgcccctcctcgggatttcgccgcataccggtcc

SEQ ID NO: 46 - protein, 41 aa, synthetic construct
RSKRSRLLHSDYMNMTPRRPGPTRKHYQPYAPPRDFAAYRS

SEQ ID NO: 47 - DNA, 336 bp, synthetic construct
  source 1..336: mol_type = other DNA
agagtgaagttcagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcgg

SEQ ID NO: 48 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - DNA, 201 bp, synthetic construct
  source 1..201: mol_type = other DNA
gcggccgcggtcggattccaagacatggaatgcgtgccctgcggcgacccgccacctccttacgagccgcactgcgcatcgaaggtcaacctcgtgaagatcgcgagcaccgcgtcctcaccccgggatactgctctggccgccgtgatttgttccgccttggccaccgtgcttctggccctgctgatcctctgtgtgatc

SEQ ID NO: 50 - protein, 67 aa, synthetic construct
AAAVGFQDMECVPCGDPPPPYEPHCASKVNLVKIASTASSPRDTALAAVICSALATVLLALLILCVI

SEQ ID NO: 51 - DNA, 63 bp, synthetic construct
  source 1..63: mol_type = other DNA
gccgccgtgatttgttccgccttggccaccgtgcttctggccctgctgatcctctgtgtgatc

SEQ ID NO: 52 - protein, 21 aa, synthetic construct
AAVICSALATVLLALLILCVI

SEQ ID NO: 53 - DNA, 138 bp, synthetic construct
  source 1..138: mol_type = other DNA
gcggccgcggtcggattccaagacatggaatgcgtgccctgcggcgacccgccacctccttacgagccgcactgcgcatcgaaggtcaacctcgtgaagatcgcgagcaccgcgtcctcaccccgggatactgctctg

SEQ ID NO: 54 - protein, 46 aa, synthetic construct
AAAVGFQDMECVPCGDPPPPYEPHCASKVNLVKIASTASSPRDTAL

SEQ ID NO: 55 - DNA, 80 bp, synthetic construct
  source 1..80: mol_type = other DNA
tacgagcctcactgcgccagcaaagtcaacttggtgaagatcgcgagcactgcctcgtcccctcgggacactgctctggc

SEQ ID NO: 56 - protein, 26 aa, synthetic construct
YEPHCASKVNLVKIASTASSPRDTAL

SEQ ID NO: 57 - DNA, 222 bp, synthetic construct
  source 1..222: mol_type = other DNA
gcggccgcgcccgcccctcggcccccgactcctgccccgacgatcgcttcccaacctctctcgctgcgcccggaagcatgccggcccgccgccggtggcgctgtccacactcgcggactggactttgataccgcactggcggccgtgatctgtagcgccctggccaccgtgctgctggcgctgctcatcctttgcgtgatctactgcaagcggcagcctagg

SEQ ID NO: 58 - protein, 74 aa, synthetic construct
AAAPAPRPPTPAPTIASQPLSLRPEACRPAAGGAVHTRGLDFDTALAAVICSALATVLLALLILCVIYCKRQPR

SEQ ID NO: 59 - DNA, 123 bp, synthetic construct; identical to SEQ ID NO: 45
  source 1..123: mol_type = other DNA

SEQ ID NO: 60 - protein, 41 aa, synthetic construct; identical to SEQ ID NO: 46

SEQ ID NO: 61 - DNA, 336 bp, synthetic construct
  source 1..336: mol_type = other DNA
cgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 62 - protein, 112 aa, synthetic construct; identical to SEQ ID NO: 48

SEQ ID NO: 63 - DNA, 93 bp, synthetic construct
  source 1..93: mol_type = other DNA
cgcgcgaaacgcagcggcagcggcgcgaccaactttagcctgctgaaacaggcgggcgatgtggaagaaaacccgggcccgcgagcaaagagg

SEQ ID NO: 64 - protein, 31 aa, synthetic construct
RAKRSGSGATNFSLLKQAGDVEENPGPRAKR

SEQ ID NO: 65 - DNA, 78 bp, synthetic construct
  source 1..78: mol_type = other DNA
agagctaaacgctctgggtctggtgaaggacgaggtagccttcttacgtgcggagacgtggaggaaaacccaggaccc

SEQ ID NO: 66 - protein, 26 aa, synthetic construct
RAKRSGSGEGRGSLLTCGDVEENPGP

SEQ ID NO: 67 - DNA, 1005 bp, synthetic construct
  source 1..1005: mol_type = other DNA
aggaaggtttgcaatggaatcggtataggggagtttaaggattcacttagcataaacgctactaatattaaacacttcaaaaactgtacgagtataagtggagatcttcacattttgccggttgcattccgaggcgattcattcacccacacgccaccgcttgacccacaagaattggatattcttaaaaccgttaaagaaataacggggtttttgctcattcaagcgtggccagaaaatcgcactgacctccatgctttcgagaacctggagattataagaggacgaactaagcagcatggtcaattctcccttgctgtggtcagcctgaacatcaccagtcttggtttgcggtccctcaaggaaatttcagatggagatgtcatcataagcggcaacaagaatttgtgctatgcaaataccataaactggaaaaaactgtttggcacttccggccagaaaaccaagattatttcaaatcggggtgagaacagctgcaaagccaccggccaggtttgtcatgccttgtgctctccggaaggctgttgggggccagaacccagggactgcgtcagttgcagaaacgtctcaagaggccgcgaatgcgttgacaagtgtaacctccttgagggtgagccacgagagtttgttgagaacagcgagtgtatacaatgtcaccctgaatgtttgccccaggctatgaatataacctgcacaggccgcgggcctgataactgcatccagtgtgctcattacatagatggacctcactgtgtgaaaacctgcccggccggagttatgggagaaaacaacactctggtgtggaaatacgctgatgcaggccacgtgtgccacctttgtcacccgaattgtacatatgggtgtaccggtcctggacttgaaggttgccctaccaatggccctaaaatacccagtatcgcaactggcatggtaggcgctcttctcttgctcttggtagttgctctcggcataggtctttttatg

SEQ ID NO: 68 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 69 - DNA, 1482 bp, synthetic construct
  source 1..1482: mol_type = other DNA
atgcttcttttggtgacttcccttttgctgtgcgagttgccacaccccgccttcctgcttattccccaggtacagctccagcagagtggcccagggctcgtgaagccaagccagacgctgtccctgacttgtgcaatttcaggggattcagtttcatcaaatagcgcggcgtggaattggattcgacaatctccttcccgagggttggaatggcttggacgaacatattacagatccaaatggtataacgactatgcggtatcagtaaagtcaagaataaccattaaccccgacacaagcaagaaccaattctctttgcagcttaactctgtcacgccagaagacacggcagtctattattgcgctcgcgaggtaacgggtgacctggaagacgcttttgacatttgggggcagggtacgatggtgacagtcagttcagggggcggtgggagtgggggagggggtagcggggggggagggtcagacattcagatgacccagtccccttcatccttgtctgcctccgtcggtgacagggtgacaataacatgcagagcaagccaaacaatctggagctatctcaactggtaccagcagcgaccaggaaaagcgccaaacctgctgatttacgctgcttcctccctccaatcaggcgtgcctagtagatttagcggtaggggctccggcaccgattttacgctcactataagctctcttcaagcagaagattttgcgacttattactgccagcagtcctatagtatacctcagactttcggacagggtaccaagttggagattaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 70 - protein, 494 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - DNA, 1485 bp, synthetic construct
  source 1..1485: mol_type = other DNA
atgcttctcctggtcacctccctgctcctctgcgaactgcctcaccctgccttccttctgattcctgacactgacattcagatgactcagaccacctcttccttgtccgcgtcactgggagacagagtgaccatctcgtgtcgcgcaagccaggatatctccaagtacctgaactggtaccaacagaagcccgacgggactgtgaagctgctgatctaccacacctcacgcctgcacagcggagtgccaagcagattctccggctccggctcgggaaccgattactcgcttaccattagcaacctcgagcaggaggacatcgctacctacttctgccagcaaggaaataccctgccctacaccttcggcggaggaaccaaattggaaatcaccggctccacgagcggctccgggaagcctggttccggggaaggctccactaagggtgaagtgaagctccaggagtccggccccggcctggtggcgccgtcgcaatcactctctgtgacctgtaccgtgtcgggagtgtccctgcctgattacggcgtgagctggattcggcagccgccgcggaagggcctggaatggctgggtgtcatctggggatccgagactacctactacaactcggccctgaagtcccgcctgactatcatcaaagacaactcgaagtcccaggtctttctgaagatgaactccctgcaaactgacgacaccgccatctattactgtgctaagcactactactacggtggaagctatgctatggactactggggccaggggacatccgtgacagtcagctccgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 72 - protein, 495 aa, synthetic construct
MLLLVTSLLLCELPHPAFLLIPDTDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - DNA, 1470 bp, synthetic construct
  source 1..1470: mol_type = other DNA
atgcttctcctggtcacctccctgctcctctgcgaactgcctcaccctgccttccttctgattcctgacattcagatgactcagaccacctcttccttgtccgcgtcactgggagacagagtgaccatctcgtgtcgcgcaagccaggatatctccaagtacctgaactggtaccaacagaagcccgacgggactgtgaagctgctgatctaccacacctcacgcctgcacagcggagtgccaagcagattctccggctccggctcgggaaccgattactcgcttaccattagcaacctcgagcaggaggacatcgctacctacttctgccagcaaggaaataccctgccctacaccttcggcggaggaaccaaattggaaatcaccggcggaggaggctccgggggaggaggttccgggggcgggggttccgaagtgaagctccaggagtccggccccggcctggtggcgccgtcgcaatcactctctgtgacctgtaccgtgtcgggagtgtccctgcctgattacggcgtgagctggattcggcagccgccgcggaagggcctggaatggctgggtgtcatctggggatccgagactacctactacaactcggccctgaagtcccgcctgactatcatcaaagacaactcgaagtcccaggtctttctgaagatgaactccctgcaaactgacgacaccgccatctattactgtgctaagcactactactacggtggaagctatgctatggactactgggggcaaggcacttcggtgactgtgtcaagcgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 74 - protein, 490 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 1467 bp, synthetic construct
  source 1..1467: mol_type = other DNA
atgctgctgctggtgaccagcctgctgctgtgcgaactgccgcatccggcgtttctgctgattccggatattcagatgacccagaccaccagcagcctgagcgcgagcctgggcgatcgcgtgaccattagctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattatagcctgaccattagcaacctggaacaggaagatattgcgacctatttttgccagcagggcaacaccctgccgtatacctttggcggcggcaccaaactggaaattaccggcggcggcggcagcggcggcggcggcagcggcggcggcggcagcgaagtgaaactgcaggaaagcggcccgggcctggtggcgccgagccagagcctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattattaaagataacagcaaaagccaggtgtttctgaaaatgaacagcctgcagaccgatgataccgcgatttattattgcgcgaaacattattattatggcggcagctatgcgatggattattggggccagggcaccagcgtgaccgtgagcagcgcggcggcgccggcgccgcgcccgccgaccccggcgccgaccattgcgagccagccgctgagcctgcgcccggaagcgtgccgcccggcggcgggcggcgcggtgcatacccgcggcctggattttgtgcagccgatggcgctgattgtgctgggcggcgtggcgggcctgctgctgtttattggcctgggcatttttttttgcgtgcgctgccgcccgcgccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 76 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAPAPRPPTPAPTIASQPLSLRPEACRPAAGGAVHTRGLDFVQPMALIVLGGVAGLLLFIGLGIFFCVRCRPRRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - DNA, 1467 bp, synthetic construct
  source 1..1467: mol_type = other DNA
atgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgcgcccgcccctcggcccccgactcctgccccgacgatcgcttcccaacctctctcgctgcgcccggaagcatgccggcccgccgccggtggcgctgtccacactcgcggactggactttgataccgcactggcggccgtgatctgtagcgccctggccaccgtgctgctggcgctgctcatcctttgcgtgatctactgcaagcggcagcctaggcgaaagaagctcctctacattttcaagcaacccttcatgcgccccgtgcaaaccacccaggaggaggatggatgctcatgccggttccctgaggaagaagagggcggttgcgagctcagagtgaaattcagccggtcggctgacgccccggcgtaccagcagggccagaaccagctgtacaatgagctcaacctggggcgccgcgaagagtacgacgtgctggacaagaggagaggcagagatccggaaatgggcggaaagccaaggcggaagaacccgcaggaaggtctttacaacgaactgcagaaggacaagatggccgaggcctactccgagattgggatgaagggagaaagacggaggggaaagggacatgacggactttaccagggcctgagcactgccacgaaggacacctatgatgccctgcacatgcaggcgctgccgcctcgg

SEQ ID NO: 78 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAPAPRPPTPAPTIASQPLSLRPEACRPAAGGAVHTRGLDFDTALAAVICSALATVLLALLILCVIYCKRQPRRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - DNA, 36 bp, Homo sapiens
  source 1..36: mol_type = genomic DNA
gagagcaaatacgggccgccatgtcccccgtgtccg

SEQ ID NO: 80 - protein, 12 aa, Homo sapiens
ESKYGPPCPPCP

SEQ ID NO: 81 - DNA, 327 bp, Homo sapiens
  source 1..327: mol_type = genomic DNA
gcaccaccagttgctggccctagtgtcttcttgttccctcccaagcccaaagacaccttgatgatttccagaactcctgaggttacctgcgttgtcgtagatgtttctcaggaggacccagaggtccaatttaactggtacgttgatggggtggaagttcacaatgcgaagacaaagccgcgggaagaacaatttcagtccacttaccgggttgtcagcgttctgacggtattgcatcaagactggcttaatggaaaggaatataagtgtaaggtgtccaacaaaggtttgccgagcagtattgagaagaccatatcaaaggcgaag

SEQ ID NO: 82 - protein, 109 aa, Homo sapiens
APPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 83 - DNA, 321 bp, Homo sapiens
  source 1..321: mol_type = genomic DNA
gggcagccgcgcgagccacaagtttacactttgccgccatctcaagaggaaatgactaaaaaccaggtatccttgacatgcctcgtaaaaggattttatccatctgatattgctgtggaatgggagtctaacgggcagccggaaaataattacaaaactacaccacctgtgctcgattcagatggaagtttcttcctttacagtagacttacggtggacaaatctaggtggcaggaagggaatgtgtttagttgtagtgtaatgcacgaggcacttcataaccactatacacagaagtcactgagtttgagtcttggcaaa

SEQ ID NO: 84 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - DNA, 684 bp, Homo sapiens
  source 1..684: mol_type = genomic DNA
gagagcaaatacgggccgccatgtcccccgtgtccggcaccaccagttgctggccctagtgtcttcttgttccctcccaagcccaaagacaccttgatgatttccagaactcctgaggttacctgcgttgtcgtagatgtttctcaggaggacccagaggtccaatttaactggtacgttgatggggtggaagttcacaatgcgaagacaaagccgcgggaagaacaatttcagtccacttaccgggttgtcagcgttctgacggtattgcatcaagactggcttaatggaaaggaatataagtgtaaggtgtccaacaaaggtttgccgagcagtattgagaagaccatatcaaaggcgaaggggcagccgcgcgagccacaagtttacactttgccgccatctcaagaggaaatgactaaaaaccaggtatccttgacatgcctcgtaaaaggattttatccatctgatattgctgtggaatgggagtctaacgggcagccggaaaataattacaaaactacaccacctgtgctcgattcagatggaagtttcttcctttacagtagacttacggtggacaaatctaggtggcaggaagggaatgtgtttagttgtagtgtaatgcacgaggcacttcataaccactatacacagaagtcactgagtttgagtcttggcaaa

SEQ ID NO: 86 - protein, 228 aa, Homo sapiens
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - DNA, 732 bp, synthetic construct
  source 1..732: mol_type = other DNA
gaggtccagctggtacagtctgggggaggcttggtacagcctggggggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatttatcgtcagtggctggaccctttaactactggggccagggcaccctggtcaccgtctcctcaggaggtggcgggtctggtggaggcggtagcggcggtggcggatcctcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaggatgaggctgactattactgtaactcccgggacagcagtggtaaccatctggtattcggcggaggcacccagctgaccgtcctcggt

SEQ ID NO: 88 - protein, 244 aa, synthetic construct
EVQLVQSGGGLVQPGGSLRLSCAASGFTFDDYAMHWVRQAPGKGLEWVSGISWNSGSIGYADSVKGRFTISRDNAKNSLYLQMNSLRAEDTALYYCAKDLSSVAGPFNYWGQGTLVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTQLTVLG

SEQ ID NO: 89 - DNA, 1476 bp, synthetic construct
  source 1..1476: mol_type = other DNA
atgctgctgctggtgaccagcctgctgctgtgcgaactgccgcatccggcgtttctgctgattccggaggtccagctggtacagtctgggggaggcttggtacagcctggggggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatttatcgtcagtggctggaccctttaactactggggccagggcaccctggtcaccgtctcctcaggaggtggcgggtctggtggaggcggtagcggcggtggcggatcctcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaggatgaggctgactattactgtaactcccgggacagcagtggtaaccatctggtattcggcggaggcacccagctgaccgtcctcggtgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 90 - protein, 492 aa, synthetic construct
MLLLVTSLLLCELPHPAFLLIPEVQLVQSGGGLVQPGGSLRLSCAASGFTFDDYAMHWVRQAPGKGLEWVSGISWNSGSIGYADSVKGRFTISRDNAKNSLYLQMNSLRAEDTALYYCAKDLSSVAGPFNYWGQGTLVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTQLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - DNA, 339 bp, synthetic construct
  source 1..339: mol_type = other DNA
gaggtgcagctggtggagtctgggggaggcttggtacagcctggagggtccctgagactctcctgtgcagcctctggattcaccttcagtagctatggcatgagctgggtccgccaggctccaagacaagggcttgagtgggtggccaacataaagcaagatggaagtgagaaatactatgcggactcagtgaagggccgattcaccatctccagagacaattccaagaacacgctgtatctgcaaatgaacagcctgagagccgaggacacagccacgtattactgtgcgaaagaaaatgtggactggggccagggcaccctggtcaccgtctcctca

SEQ ID NO: 92 - protein, 113 aa, synthetic construct
EVQLVESGGGLVQPGGSLRLSCAASGFTFSSYGMSWVRQAPRQGLEWVANIKQDGSEKYYADSVKGRFTISRDNSKNTLYLQMNSLRAEDTATYYCAKENVDWGQGTLVTVSS

SEQ ID NO: 93 - DNA, 1083 bp, synthetic construct
  source 1..1083: mol_type = other DNA
atgctgctgctggtgaccagcctgctgctgtgcgaactgccgcatccggcgtttctgctgattccggaggtgcagctggtggagtctgggggaggcttggtacagcctggagggtccctgagactctcctgtgcagcctctggattcaccttcagtagctatggcatgagctgggtccgccaggctccaagacaagggcttgagtgggtggccaacataaagcaagatggaagtgagaaatactatgcggactcagtgaagggccgattcaccatctccagagacaattccaagaacacgctgtatctgcaaatgaacagcctgagagccgaggacacagccacgtattactgtgcgaaagaaaatgtggactggggccagggcaccctggtcaccgtctcctcagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 94 - protein, 361 aa, synthetic construct
MLLLVTSLLLCELPHPAFLLIPEVQLVESGGGLVQPGGSLRLSCAASGFTFSSYGMSWVRQAPRQGLEWVANIKQDGSEKYYADSVKGRFTISRDNSKNTLYLQMNSLRAEDTATYYCAKENVDWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - DNA, 2283 bp, synthetic construct
  source 1..2283: mol_type = other DNA
atgctccttctcgtgacctccctgcttctctgcgaactgccccatcctgccttcctgctgattcccgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccggaggcggcggcagcggcgggggagggtccggagggggtggttctggtggaggaggatcgggaggcggtggcagcgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 96 - protein, 761 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSGGGGSGGGGSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - DNA, 2283 bp, synthetic construct
  source 1..2283: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 98 - protein, 761 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - DNA, 797 bp, synthetic construct
  source 1..797: mol_type = other DNA
atggcactgcccgtgaccgccctgcttctgccgcttgcacttctgctgcacgccgctaggccccaagtcaccctcaaagagtcagggccaggaatcctcaagccctcacagactctgtctcttacttgctcattcagcggattcagcctttccacctctggtatgggcgtggggtggattaggcaacctagcggaaaggggcttgaatggctggcccacatctggtgggacgacgacaagtactacaacccctcactgaagtcccagctcactatttccaaagatacttcccggaatcaggtgttcctcaagattacctctgtcgacaccgctgataccgccacttactattgttcacgcagaccgagaggtaccatggacgcaatggactactggggacagggcaccagcgtgaccgtgtcatctggcggtggagggtcaggaggtggaggtagcggaggcggtgggtccgacattgtcatgacccaggccgccagcagcctgagcgcttcactgggcgacagggtgaccatcagctgtcgcgcatcacaagatatctctaagtatcttaattggtaccagcaaaagccggatggaaccgtgaagctgctgatctactacacctcacggctgcattctggagtgcctagccgctttagcggatctgggtccggtactgactacagcctcaccattagaaaccttgaacaggaggacatcgcaacttatttctgccaacaggtctatactctgccgtggaccttcggcggaggtaccaaactggagattaagtccgg

SEQ ID NO: 100 - protein, 265 aa, synthetic construct
MALPVTALLLPLALLLHAARPQVTLKESGPGILKPSQTLSLTCSFSGFSLSTSGMGVGWIRQPSGKGLEWLAHIWWDDDKYYNPSLKSQLTISKDTSRNQVFLKITSVDTADTATYYCSRRPRGTMDAMDYWGQGTSVTVSSGGGGSGGGGSGGGGSDIVMTQAASSLSASLGDRVTISCRASQDISKYLNWYQQKPDGTVKLLIYYTSRLHSGVPSRFSGSGSGTDYSLTIRNLEQEDIATYFCQQVYTLPWTFGGGTKLEIKS

SEQ ID NO: 101 - DNA, 1170 bp, synthetic construct
  source 1..1170: mol_type = other DNA
atggcactgcccgtgaccgccctgcttctgccgcttgcacttctgctgcacgccgctaggccccaagtcaccctcaaagagtcagggccaggaatcctcaagccctcacagactctgtctcttacttgctcattcagcggattcagcctttccacctctggtatgggcgtggggtggattaggcaacctagcggaaaggggcttgaatggctggcccacatctggtgggacgacgacaagtactacaacccctcactgaagtcccagctcactatttccaaagatacttcccggaatcaggtgttcctcaagattacctctgtcgacaccgctgataccgccacttactattgttcacgcagaccgagaggtaccatggacgcaatggactactggggacagggcaccagcgtgaccgtgtcatctggcggtggagggtcaggaggtggaggtagcggaggcggtgggtccgacattgtcatgacccaggccgccagcagcctgagcgcttcactgggcgacagggtgaccatcagctgtcgcgcatcacaagatatctctaagtatcttaattggtaccagcaaaagccggatggaaccgtgaagctgctgatctactacacctcacggctgcattctggagtgcctagccgctttagcggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 102 - protein, 490 aa, synthetic construct
MALPVTALLLPLALLLHAARPQVTLKESGPGILKPSQTLSLTCSFSGFSLSTSGMGVGWIRQPSGKGLEWLAHIWWDDDKYYNPSLKSQLTISKDTSRNQVFLKITSVDTADTATYYCSRRPRGTMDAMDYWGQGTSVTVSSGGGGSGGGGSGGGGSDIVMTQAASSLSASLGDRVTISCRASQDISKYLNWYQQKPDGTVKLLIYYTSRLHSGVPSRFSGSGSGTDYSLTIRNLEQEDIATYFCQQVYTLPWTFGGGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - DNA, 2366 bp, synthetic construct
  source 1..2366: mol_type = other DNA
tgagctctgagtcagtgactcagtgagtcagtgactcagtgagtcagtgactcagctcgaggatctcgctagcgggaatttccggggactttccgggaatttccggggactttccgggaatttccagatctggcctcggcggccaagcttagacactagagggtatataatggaagctcgacttccagcttggcaatccggtactgttggtaaaggcgcgccccaccatgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgctaccacaacccctgcgccccggcctcctacccccgcacccacgattgcttctcaacctctttcactccgacctgaggcttgtagacctgcagccgggggtgccgtccacacacggggactcgacttcgcttgtgatatatatatttgggcgcccctggccggcacttgtggagttcttttgctctctcttgttatcacattgtactgcaagcgaggtaggaagaaattgctttacatttttaagcagccgttcatgcgaccagtacagactactcaagaagaagatgggtgctcttgtcggttcccggaagaagaagagggtggttgcgagttgagggtgaagttctcccgctctgccgacgcaccggcatatcagcagggacaaaaccagctctacaacgaattgaacctgggtcggcgggaagaatatgacgtgctcgataagcggcggggtcgcgacccagaaatgggaggcaaaccgcgcaggaaaaatccacaggagggactttataacgaacttcaaaaggataagatggcagaggcatacagcgaaatcgggatgaaaggcgagagaagaagggggaaagggcacgatggtctttaccaggggctttctaccgcgacgaaggatacctacgatgctctccatatgcaagcacttcctcctagacgggcaaagcggggctcaggggcgactaacttttcactgttgaagcaggccggggatgtggaggagaatcctggtcctagagctaagcgagtagacatgggaagagggctgctccgaggcttgtggccgttgcatattgtattgtggacgcggatagcgagtacaatcccgcctcacgtgcaaaaatcagttaataacgacatgatcgttactgacaacaatggcgcagttaaatttccgcagctttgtaaattctgtgatgtaagattttcaacgtgcgataaccagaaaagctgtatgtccaactgcagcatcacatcaatctgtgaaaaaccccaagaggtatgtgtggccgtctggcgaaagaatgacgaaaatatcacactggagaccgtttgtcacgatcctaaactcccttatcatgactttattctggaagacgcagcgtcaccgaagtgtataatgaaagagaagaagaagcctggagagacgtttttcatgtgcagttgctcctcagatgagtgtaatgacaacatcattttttccgaggagtacaatacgagtaacccagacctcctgctggttattttccaggtaaccggcatcagtttgttgcccccactgggtgttgcaatcagtgtaataatcatattttattgttaccgggtg

SEQ ID NO: 104 - protein, 713 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRVDMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRV

SEQ ID NO: 105 - DNA, 2390 bp, synthetic construct
  source 1..2390: mol_type = other DNA
tgagctctgagtcagtgactcagtgagtcagtgactcagtgagtcagtgactcagctcgaggatctcgctagcgggaatttccggggactttccgggaatttccggggactttccgggaatttccagatctggcctcggcggccaagcttagacactagagggtatataatggaagctcgacttccagcttggcaatccggtactgttggtaaaggcgcgccccaccatgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgctaccacaacccctgcgccccggcctcctacccccgcacccacgattgcttctcaacctctttcactccgacctgaggcttgtagacctgcagccgggggtgccgtccacacacggggactcgacttcgcttgtgatatatatatttgggcgcccctggccggcacttgtggagttcttttgctctctcttgttatcacattgtactgcaagcgaggtaggaagaaattgctttacatttttaagcagccgttcatgcgaccagtacagactactcaagaagaagatgggtgctcttgtcggttcccggaagaagaagagggtggttgcgagttgagggtgaagttctcccgctctgccgacgcaccggcatatcagcagggacaaaaccagctctacaacgaattgaacctgggtcggcgggaagaatatgacgtgctcgataagcggcggggtcgcgacccagaaatgggaggcaaaccgcgcaggaaaaatccacaggagggactttataacgaacttcaaaaggataagatggcagaggcatacagcgaaatcgggatgaaaggcgagagaagaagggggaaagggcacgatggtctttaccaggggctttctaccgcgacgaaggatacctacgatgctctccatatgcaagcacttcctcctagacgggcaaagcggggctcaggggcgactaacttttcactgttgaagcaggccggggatgtggaggagaatcctggtcctagagctaagcgagtagacatgcaaataccacaagcgccgtggcccgtcgtctgggctgttcttcagcttggctggagacctggctggtttctcgattctccagataggccctggaacccaccgacgttcagtccggcacttctcgtggtcacagagggggacaacgccacttttacgtgctcctttagcaacacctccgagtccttcgtactgaattggtatcgcatgagtccgtccaatcagacggacaagttggccgcgtttcctgaagataggtcacaacctggccaagactgtcggttccgggtcacccagttgccgaatgggagggactttcatatgagcgtcgttcgggcaagaaggaacgattctggaacgtatttgtgcggggccattagccttgcgccaaaagcgcagataaaggagtcccttcgggcagaacttcgcgtgacagagcgacgcgctgaagttccgactgcccatccctctccgagcccacgaccagccggacaatttcagactcttgtggtgggtgtggtaggcggcctgctcggaagcttggtcttgctggtgtgggtcctggctgtgatttgcagtcgagcagcccgcggcacc

SEQ ID NO: 106 - protein, 721 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRVDMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGT

SEQ ID NO: 107 - DNA, 3050 bp, synthetic construct
  source 1..3050: mol_type = other DNA
tgagctctgagtcagtgactcagtgagtcagtgactcagtgagtcagtgactcagctcgaggatctcgctagcgggaatttccggggactttccgggaatttccggggactttccgggaatttccagatctggcctcggcggccaagcttagacactagagggtatataatggaagctcgacttccagcttggcaatccggtactgttggtaaaggcgcgccccaccatgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgctaccacaacccctgcgccccggcctcctacccccgcacccacgattgcttctcaacctctttcactccgacctgaggcttgtagacctgcagccgggggtgccgtccacacacggggactcgacttcgcttgtgatatatatatttgggcgcccctggccggcacttgtggagttcttttgctctctcttgttatcacattgtactgcaagcgaggtaggaagaaattgctttacatttttaagcagccgttcatgcgaccagtacagactactcaagaagaagatgggtgctcttgtcggttcccggaagaagaagagggtggttgcgagttgagggtgaagttctcccgctctgccgacgcaccggcatatcagcagggacaaaaccagctctacaacgaattgaacctgggtcggcgggaagaatatgacgtgctcgataagcggcggggtcgcgacccagaaatgggaggcaaaccgcgcaggaaaaatccacaggagggactttataacgaacttcaaaaggataagatggcagaggcatacagcgaaatcgggatgaaaggcgagagaagaagggggaaagggcacgatggtctttaccaggggctttctaccgcgacgaaggatacctacgatgctctccatatgcaagcacttcctcctagacgggcaaagcggggctcaggggcgactaacttttcactgttgaagcaggccggggatgtggaggagaatcctggtcctagagctaagcgagtagacatgcaaataccacaagcgccgtggcccgtcgtctgggctgttcttcagcttggctggagacctggctggtttctcgattctccagataggccctggaacccaccgacgttcagtccggcacttctcgtggtcacagagggggacaacgccacttttacgtgctcctttagcaacacctccgagtccttcgtactgaattggtatcgcatgagtccgtccaatcagacggacaagttggccgcgtttcctgaagataggtcacaacctggccaagactgtcggttccgggtcacccagttgccgaatgggagggactttcatatgagcgtcgttcgggcaagaaggaacgattctggaacgtatttgtgcggggccattagccttgcgccaaaagcgcagataaaggagtcccttcgggcagaacttcgcgtgacagagcgacgcgctgaagttccgactgcccatccctctccgagcccacgaccagccggacaatttcagactcttgtggtgggtgtggtaggcggcctgctcggaagcttggtcttgctggtgtgggtcctggctgtgatttgcagtcgagcagcccgcggcacccgggctaaacggagcggctccggggcaaccaactttagtctgctgaaacaggcaggagatgtggaggaaaacccaggaccagctagcatgggaagagggctgctccgaggcttgtggccgttgcatattgtattgtggacgcggatagcgagtacaatcccgcctcacgtgcaaaaatcagttaataacgacatgatcgttactgacaacaatggcgcagttaaatttccgcagctttgtaaattctgtgatgtaagattttcaacgtgcgataaccagaaaagctgtatgtccaactgcagcatcacatcaatctgtgaaaaaccccaagaggtatgtgtggccgtctggcgaaagaatgacgaaaatatcacactggagaccgtttgtcacgatcctaaactcccttatcatgactttattctggaagacgcagcgtcaccgaagtgtataatgaaagagaagaagaagcctggagagacgtttttcatgtgcagttgctcctcagatgagtgtaatgacaacatcattttttccgaggagtacaatacgagtaacccagacctcctgctggttattttccaggtaaccggcatcagtttgttgcccccactgggtgttgcaatcagtgtaataatcatattttattgttaccgggtg

SEQ ID NO: 108 - protein, 941 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRVDMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTRAKRSGSGATNFSLLKQAGDVEENPGPASMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRV

SEQ ID NO: 109 - DNA, 2342 bp, synthetic construct
  source 1..2342: mol_type = other DNA
ggtacctgagctcagttctgagaaaagtagttctgagaaaagtagttctgagaaaagtagttctgagaaaagtagttctgagaaaagtctcgaggatatcaagatctggcctcggcggccaagcttagacactagagggtatataatggaagctcgacttccagcttggcaatccggtactgttggtaaaggcgcgccccaccatgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgctaccacaacccctgcgccccggcctcctacccccgcacccacgattgcttctcaacctctttcactccgacctgaggcttgtagacctgcagccgggggtgccgtccacacacggggactcgacttcgcttgtgatatatatatttgggcgcccctggccggcacttgtggagttcttttgctctctcttgttatcacattgtactgcaagcgaggtaggaagaaattgctttacatttttaagcagccgttcatgcgaccagtacagactactcaagaagaagatgggtgctcttgtcggttcccggaagaagaagagggtggttgcgagttgagggtgaagttctcccgctctgccgacgcaccggcatatcagcagggacaaaaccagctctacaacgaattgaacctgggtcggcgggaagaatatgacgtgctcgataagcggcggggtcgcgacccagaaatgggaggcaaaccgcgcaggaaaaatccacaggagggactttataacgaacttcaaaaggataagatggcagaggcatacagcgaaatcgggatgaaaggcgagagaagaagggggaaagggcacgatggtctttaccaggggctttctaccgcgacgaaggatacctacgatgctctccatatgcaagcacttcctcctagacgggcaaagcggggctcaggggcgactaacttttcactgttgaagcaggccggggatgtggaggagaatcctggtcctagagctaagcgagtagacatgggaagagggctgctccgaggcttgtggccgttgcatattgtattgtggacgcggatagcgagtacaatcccgcctcacgtgcaaaaatcagttaataacgacatgatcgttactgacaacaatggcgcagttaaatttccgcagctttgtaaattctgtgatgtaagattttcaacgtgcgataaccagaaaagctgtatgtccaactgcagcatcacatcaatctgtgaaaaaccccaagaggtatgtgtggccgtctggcgaaagaatgacgaaaatatcacactggagaccgtttgtcacgatcctaaactcccttatcatgactttattctggaagacgcagcgtcaccgaagtgtataatgaaagagaagaagaagcctggagagacgtttttcatgtgcagttgctcctcagatgagtgtaatgacaacatcattttttccgaggagtacaatacgagtaacccagacctcctgctggttattttccaggtaaccggcatcagtttgttgcccccactgggtgttgcaatcagtgtaataatcatattttattgttaccgggtg

SEQ ID NO: 110 - DNA, 2366 bp, synthetic construct
  source 1..2366: mol_type = other DNA
ggtacctgagctcagttctgagaaaagtagttctgagaaaagtagttctgagaaaagtagttctgagaaaagtagttctgagaaaagtctcgaggatatcaagatctggcctcggcggccaagcttagacactagagggtatataatggaagctcgacttccagcttggcaatccggtactgttggtaaaggcgcgccccaccatgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgctaccacaacccctgcgccccggcctcctacccccgcacccacgattgcttctcaacctctttcactccgacctgaggcttgtagacctgcagccgggggtgccgtccacacacggggactcgacttcgcttgtgatatatatatttgggcgcccctggccggcacttgtggagttcttttgctctctcttgttatcacattgtactgcaagcgaggtaggaagaaattgctttacatttttaagcagccgttcatgcgaccagtacagactactcaagaagaagatgggtgctcttgtcggttcccggaagaagaagagggtggttgcgagttgagggtgaagttctcccgctctgccgacgcaccggcatatcagcagggacaaaaccagctctacaacgaattgaacctgggtcggcgggaagaatatgacgtgctcgataagcggcggggtcgcgacccagaaatgggaggcaaaccgcgcaggaaaaatccacaggagggactttataacgaacttcaaaaggataagatggcagaggcatacagcgaaatcgggatgaaaggcgagagaagaagggggaaagggcacgatggtctttaccaggggctttctaccgcgacgaaggatacctacgatgctctccatatgcaagcacttcctcctagacgggcaaagcggggctcaggggcgactaacttttcactgttgaagcaggccggggatgtggaggagaatcctggtcctagagctaagcgagtagacatgcaaataccacaagcgccgtggcccgtcgtctgggctgttcttcagcttggctggagacctggctggtttctcgattctccagataggccctggaacccaccgacgttcagtccggcacttctcgtggtcacagagggggacaacgccacttttacgtgctcctttagcaacacctccgagtccttcgtactgaattggtatcgcatgagtccgtccaatcagacggacaagttggccgcgtttcctgaagataggtcacaacctggccaagactgtcggttccgggtcacccagttgccgaatgggagggactttcatatgagcgtcgttcgggcaagaaggaacgattctggaacgtatttgtgcggggccattagccttgcgccaaaagcgcagataaaggagtcccttcgggcagaacttcgcgtgacagagcgacgcgctgaagttccgactgcccatccctctccgagcccacgaccagccggacaatttcagactcttgtggtgggtgtggtaggcggcctgctcggaagcttggtcttgctggtgtgggtcctggctgtgatttgcagtcgagcagcccgcggcacc

SEQ ID NO: 111 - DNA, 3026 bp, synthetic construct
  source 1..3026: mol_type = other DNA
ggtacctgagctcagttctgagaaaagtagttctgagaaaagtagttctgagaaaagtagttctgagaaaagtagttctgagaaaagtctcgaggatatcaagatctggcctcggcggccaagcttagacactagagggtatataatggaagctcgacttccagcttggcaatccggtactgttggtaaaggcgcgccccaccatgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgctaccacaacccctgcgccccggcctcctacccccgcacccacgattgcttctcaacctctttcactccgacctgaggcttgtagacctgcagccgggggtgccgtccacacacggggactcgacttcgcttgtgatatatatatttgggcgcccctggccggcacttgtggagttcttttgctctctcttgttatcacattgtactgcaagcgaggtaggaagaaattgctttacatttttaagcagccgttcatgcgaccagtacagactactcaagaagaagatgggtgctcttgtcggttcccggaagaagaagagggtggttgcgagttgagggtgaagttctcccgctctgccgacgcaccggcatatcagcagggacaaaaccagctctacaacgaattgaacctgggtcggcgggaagaatatgacgtgctcgataagcggcggggtcgcgacccagaaatgggaggcaaaccgcgcaggaaaaatccacaggagggactttataacgaacttcaaaaggataagatggcagaggcatacagcgaaatcgggatgaaaggcgagagaagaagggggaaagggcacgatggtctttaccaggggctttctaccgcgacgaaggatacctacgatgctctccatatgcaagcacttcctcctagacgggcaaagcggggctcaggggcgactaacttttcactgttgaagcaggccggggatgtggaggagaatcctggtcctagagctaagcgagtagacatgcaaataccacaagcgccgtggcccgtcgtctgggctgttcttcagcttggctggagacctggctggtttctcgattctccagataggccctggaacccaccgacgttcagtccggcacttctcgtggtcacagagggggacaacgccacttttacgtgctcctttagcaacacctccgagtccttcgtactgaattggtatcgcatgagtccgtccaatcagacggacaagttggccgcgtttcctgaagataggtcacaacctggccaagactgtcggttccgggtcacccagttgccgaatgggagggactttcatatgagcgtcgttcgggcaagaaggaacgattctggaacgtatttgtgcggggccattagccttgcgccaaaagcgcagataaaggagtcccttcgggcagaacttcgcgtgacagagcgacgcgctgaagttccgactgcccatccctctccgagcccacgaccagccggacaatttcagactcttgtggtgggtgtggtaggcggcctgctcggaagcttggtcttgctggtgtgggtcctggctgtgatttgcagtcgagcagcccgcggcacccgggctaaacggagcggctccggggcaaccaactttagtctgctgaaacaggcaggagatgtggaggaaaacccaggaccagctagcatgggaagagggctgctccgaggcttgtggccgttgcatattgtattgtggacgcggatagcgagtacaatcccgcctcacgtgcaaaaatcagttaataacgacatgatcgttactgacaacaatggcgcagttaaatttccgcagctttgtaaattctgtgatgtaagattttcaacgtgcgataaccagaaaagctgtatgtccaactgcagcatcacatcaatctgtgaaaaaccccaagaggtatgtgtggccgtctggcgaaagaatgacgaaaatatcacactggagaccgtttgtcacgatcctaaactcccttatcatgactttattctggaagacgcagcgtcaccgaagtgtataatgaaagagaagaagaagcctggagagacgtttttcatgtgcagttgctcctcagatgagtgtaatgacaacatcattttttccgaggagtacaatacgagtaacccagacctcctgctggttattttccaggtaaccggcatcagtttgttgcccccactgggtgttgcaatcagtgtaataatcatattttattgttaccgggtg

SEQ ID NO: 112 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 113 - DNA, 729 bp, synthetic construct
  source 1..729: mol_type = other DNA
gaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaa

SEQ ID NO: 114 - protein, 243 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 115 - DNA, 1491 bp, synthetic construct
  source 1..1491: mol_type = other DNA
atgctgctgctggtgaccagcctgctgctgtgcgaactgccgcatccggcgtttctgctgattccgcaagtgcaactcgtccaatccggtgccgaagtcaagaagcctggttcctccgtgaaagtgtcgtgcaaagccagcggcgggacttttagctcctacgcgatcagctgggtcagacaggcccctggacaaggcctcgagtggatgggcggcatcattccgattttcggtaccgccaactacgcccagaagttccagggacgcgtgaccattactaccgacgagagcacctcaaccgcatacatggaactgtccagcctgcgctccgaggacacggctgtgtactattgcgccagacggggatggggaggattctcctccggctccgcattcgacatctggggacagggcactatggtcactgtgtcatccgggggaggaggatcaggcggtggaggatccggtggtggcggatccaacttcatgctgacccagccccactcagtgtcggaatcgcccggcaacaccgtgactatcagctgcaccggatccagcgggaccatcggctctaatttcgtgcagtggtaccagcagtccccagggagagctccgaccctgttgatctacgaggacacaaagcggccaagcggagtgccgcctagattcgccggctccgtggattcctcgtccaactcggcgtcgctgaccatcagcggactcaagactgaagatgaagccgactactactgtcagtcctacgactcgagcaactgggtgtttgggggcgggactaagctgaccgtgcttggagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 116 - protein, 497 aa, synthetic construct
MLLLVTSLLLCELPHPAFLLIPQVQLVQSGAEVKKPGSSVKVSCKASGGTFSSYAISWVRQAPGQGLEWMGGIIPIFGTANYAQKFQGRVTITTDESTSTAYMELSSLRSEDTAVYYCARRGWGGFSSGSAFDIWGQGTMVTVSSGGGGSGGGGSGGGGSNFMLTQPHSVSESPGNTVTISCTGSSGTIGSNFVQWYQQSPGRAPTLLIYEDTKRPSGVPPRFAGSVDSSSNSASLTISGLKTEDEADYYCQSYDSSNWVFGGGTKLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - DNA, 1479 bp, synthetic construct
  source 1..1479: mol_type = other DNA
atgctgctgctggtgaccagcctgctgctgtgcgaactgccgcatccggcgtttctgctgattccggaagtccaattggtgcagagcggatccgaacttaagaaacctggcgcgagcgtgaaagtgtcctgcaaggcctccggagggactttctcgtcgtacgccattagctgggtccgccaagctcctggccaaggcctggagtggatgggcgggattatccccatcttcgggactgcgaactacgcccagaagtttcagggccgggtcactatcaccgccgacgaatcaacctcgaccgcctacatggaactgtcctcgcttcggtccgaggatactgccgtgtactattgtgcctcaacggccagacgcggatgggacaccgctggtccgctcgattactggggccagggaaccctcgtgaccgtcagctccggaggaggaggctccggtggtggaggatccgggggtggtggatccgacatccaaatgacccagtccccctcgtccctgagcgcctctgtgggcgacagagtgacaattgcatgcagggcctcacagactatctcccgctacctgaactggtaccagcagaagccaggaaaggcccctaagctgctcatctacgctgcgtcctcgctccaatccggggtgtcctcacggttttccggatcgggttccggcaccgagttcaccctgaccatcagcagcctgcagcccgaggacttcgcaacctacttctgccagcaaacctactccccgccgattacgttcggacaggggactcggctggaaatcaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 118 - protein, 493 aa, synthetic construct
MLLLVTSLLLCELPHPAFLLIPEVQLVQSGSELKKPGASVKVSCKASGGTFSSYAISWVRQAPGQGLEWMGGIIPIFGTANYAQKFQGRVTITADESTSTAYMELSSLRSEDTAVYYCASTARRGWDTAGPLDYWGQGTLVTVSSGGGGSGGGGSGGGGSDIQMTQSPSSLSASVGDRVTIACRASQTISRYLNWYQQKPGKAPKLLIYAASSLQSGVSSRFSGSGSGTEFTLTISSLQPEDFATYFCQQTYSPPITFGQGTRLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - DNA, 1473 bp, synthetic construct
  source 1..1473: mol_type = other DNA
atgctgctgctggtgaccagcctgctgctgtgcgaactgccgcatccggcgtttctgctgattccggaagtgcaactcgtcgaaacaggggcagaagtgaaaaacccaggctcaagcgtgaaagtgtcgtgcaaggcttcgggcggaactctgtccaactacgccatctcctgggtccgccaagctccgggaaagggcctcgagtggatgggcggaatcattcccattttcgggaccgccaactacgcgcaaaagttccagggccgggtcactatcaccgcggacgaaagcaccagcaccgcctacatggaactgtcctccctgcgctccgaggacactgccgtgtactattgcgcccggaggtcatcgtggtaccccgagggctgcttccagcactggggacagggcactctcgtgaccgtgtcgtcgggtggtggtggatcaggagggggaggatccggaggaggcggaagcgatattcagctgacccagtcaccgagctccctgtccgcctccaccggagacagagtgaccatcacgtgtcgggcctcccaagggatctcctcctacctggcctggtaccagcagaagcctggaaaggcaccgaagttgctgatctacgccgcgagcacccttcagtccggagtgcctagccgcttctcgggttccggctctggcactgacttcactctgaccattagctgcctgcagtccgaggattttgccacctactactgccagcagtactatagctaccccctgaccttcgggggcggaaccaagctcgacatcaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 120 - protein, 491 aa, synthetic construct
MLLLVTSLLLCELPHPAFLLIPEVQLVETGAEVKNPGSSVKVSCKASGGTLSNYAISWVRQAPGKGLEWMGGIIPIFGTANYAQKFQGRVTITADESTSTAYMELSSLRSEDTAVYYCARRSSWYPEGCFQHWGQGTLVTVSSGGGGSGGGGSGGGGSDIQLTQSPSSLSASTGDRVTITCRASQGISSYLAWYQQKPGKAPKLLIYAASTLQSGVPSRFSGSGSGTDFTLTISCLQSEDFATYYCQQYYSYPLTFGGGTKLDIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - DNA, 1476 bp, synthetic construct
  source 1..1476: mol_type = other DNA
atgctgctgctggtgaccagcctgctgctgtgcgaactgccgcatccggcgtttctgctgattccggaagtgcaactcgtccaatctggtgccgaagtcaagaagcctggctcaagcgtgaaagtgtcctgcaaagcgtcgggagggaccttcagctcctacgccatttcctgggtccgccaagcaccaggacagggcctggagtggatgggcggcatcatcccgatcttcgggactgccaactacgcccagaagttccaggggagagtgaccattaccgccgacgagtcgaccagcacggcctacatggaactgtccagcctgcgctccgaggacactgccgtgtactactgcgcgagggccagactcggtggagcgttcgacatctggggacagggcaccatggtcaccgtgtcatccggtggcggaggatccggtggtggcggatcaggagggggaggatcccagtccgtgctgactcagcctccctccgtgagcgctgcaccgggacagaaggtcaccatctcatgctcggggggaagctccaacatcgggaaccactacgtgtcctggtaccaacagttgcctggtgccgctccaaagctgctgatctatgacgataacaagcggccgtccggaatccccgaccggttctcggggtctagatccggaaccagcgcaactctcggcattaccggactgcagagcggcgatgaggccgactactactgtggcacatgggactcgtcgctggctgcccacgtgtttggcactggcaccaaggtcaccgtgcttggagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 122 - protein, 492 aa, synthetic construct
MLLLVTSLLLCELPHPAFLLIPEVQLVQSGAEVKKPGSSVKVSCKASGGTFSSYAISWVRQAPGQGLEWMGGIIPIFGTANYAQKFQGRVTITADESTSTAYMELSSLRSEDTAVYYCARARLGGAFDIWGQGTMVTVSSGGGGSGGGGSGGGGSQSVLTQPPSVSAAPGQKVTISCSGGSSNIGNHYVSWYQQLPGAAPKLLIYDDNKRPSGIPDRFSGSRSGTSATLGITGLQSGDEADYYCGTWDSSLAAHVFGTGTKVTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1491 bp, synthetic construct
  source 1..1491: mol_type = other DNA
atgctgctgctggtgaccagcctgctgctgtgcgaactgccgcatccggcgtttctgctgattccggaagtgcaacttgtccagagcggagccgaagtgaagaaacctggatcctccgtcaaagtgtcgtgcaaggcttcgggcggaaccttctcctcgtacgcgatctcatgggtcagacaggcacccggacagggactggagtggatgggcggcatcattcccatcttcggcaccgctaattacgcccagaagtttcaggggagagtgaccatcaccgccgacgagtccacctccactgcctacatggaactgtcctcactgaggtccgaggatactgccgtgtactactgcgcgtcgcaaaaggggggtggatggtccattgacgccttcgatatttggggacaggggacgatggtcacagtgtcatccggcggtggtggatccggtggtggcggatccggaggaggaggcagccagtccgtgctgacccagccgcctagcgtgtcggccgcatctgggcagcgcgtgaccatttcctgttccgggtcctcgtccaacatcggcaacaactacgcctcctggtaccaacagctcccgggaatggcccctaagctgctgatctacgaggacaacaagcggccatccgggatctcagaccggttcagcggatcccagtccggcacttccgcgagcctcgccatcaccggactgcaggctgaggacgaagccgactactactgccaatcatatgacagctcgctcagcggcgatgtggtgttcggcggtggcactaagctgaccgtgttgggagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 124 - protein, 497 aa, synthetic construct
MLLLVTSLLLCELPHPAFLLIPEVQLVQSGAEVKKPGSSVKVSCKASGGTFSSYAISWVRQAPGQGLEWMGGIIPIFGTANYAQKFQGRVTITADESTSTAYMELSSLRSEDTAVYYCASQKGGGWSIDAFDIWGQGTMVTVSSGGGGSGGGGSGGGGSQSVLTQPPSVSAASGQRVTISCSGSSSNIGNNYASWYQQLPGMAPKLLIYEDNKRPSGISDRFSGSQSGTSASLAITGLQAEDEADYYCQSYDSSLSGDVVFGGGTKLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - DNA, 1479 bp, synthetic construct
  source 1..1479: mol_type = other DNA
atgctgctgctggtgaccagcctgctgctgtgcgaactgccgcatccggcgtttctgctgattccgcaagtccagttgcaacaatggggagcaggccttctgaaaccgtccgagacactgagcctgacgtgcgccgtctatggcggatcgttctccggatactactggtcgtggatcagacagcctccgggaaagggtctggaatggatcggcgaaatcaaccacagcggcagcaccaattacaacccgtcactgaagtcaagggtcaccattagcgtggacacttccaagaaccagttctccctgaaactgtcgagcgtgaccgctgccgatactgccgtgtactactgtgcccgcggccaagtcaagtatagctcaagcctcggctactggggccagggaaccctcgtgaccgtgtcctcgggtggaggaggctccggtggtggaggatccggtggcggaggatcgcagtccgtgctgacccagcctccctccgtgtctgctgcccctgggcaaaaggtcaccatttcgtgctccggctcatcgtccaacatcgggaacaactttgtgtcctggtaccagcagctgcccggtactgccccaaagctgctgatctacgaggacaacaagcgcccatccgggattccggatcggttcagcggatcacggtccggaactagcgcgaccctggggatcaccgggctccagactggcgacgaagcggactactactgcggaacttgggactcctccttgggggcctgggtgttcggcggagggaccaagctcaccgtgcttggagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 126 - protein, 493 aa, synthetic construct
MLLLVTSLLLCELPHPAFLLIPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGQVKYSSSLGYWGQGTLVTVSSGGGGSGGGGSGGGGSQSVLTQPPSVSAAPGQKVTISCSGSSSNIGNNFVSWYQQLPGTAPKLLIYEDNKRPSGIPDRFSGSRSGTSATLGITGLQTGDEADYYCGTWDSSLGAWVFGGGTKLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - DNA, 1467 bp, synthetic construct
  source 1..1467: mol_type = other DNA
atgctgctgctggtgaccagcctgctgctgtgcgaactgccgcatccggcgtttctgctgattccgcaagtccaactcgtcgaaactggtggtggcctcgtgaagcctggaggatccctgcgcctttcctgtgccgcttccggctttactttctcgtcgtactccatgaactgggtcagacaggctcccggaaagggcctggaatgggtgtcctccatctcgtcctcatcctcctacatctattacgcggactccgtgaagggcagattcaccatttcccgggacaacgccaagaacagcttgtacctccaaatgaactccctgcgggcagaggacaccgccgtgtactactgcgcgagggatggggatttctggagcggagccatcgactactggggccagggaactctcgtgaccgtcagctccggtggtggtggaagcggaggcggaggttctggggggggaggatcagacattcagctgacccagtcgccatcctccctgagcgcctcagtgggggaccgcgtgactattacatgccaggcctcccaagatatctcgaactacctgaactggtatcagcagaagcctggaaaggccccgaagctgttgatctacgatgccagcaacctggagactggggtgccttcccggttctcgggatcaggctcgggcaccgatttcaccttcacgatcagcagcctgcagcccgaggacattgcaacctactactgccagcagtacgacaatctgccgctttttgggggaggcaccaagctggaaatcaaagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 128 - protein, 489 aa, synthetic construct
MLLLVTSLLLCELPHPAFLLIPQVQLVETGGGLVKPGGSLRLSCAASGFTFSSYSMNWVRQAPGKGLEWVSSISSSSSYIYYADSVKGRFTISRDNAKNSLYLQMNSLRAEDTAVYYCARDGDFWSGAIDYWGQGTLVTVSSGGGGSGGGGSGGGGSDIQLTQSPSSLSASVGDRVTITCQASQDISNYLNWYQQKPGKAPKLLIYDASNLETGVPSRFSGSGSGTDFTFTISSLQPEDIATYYCQQYDNLPLFGGGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 1467 bp, synthetic construct
  source 1..1467: mol_type = other DNA
atgctgctgctggtgaccagcctgctgctgtgcgaactgccgcatccggcgtttctgctgattccggaggtgcaattggtgcagtcaggtggtggagtggtgcagccaggaagatcccttagactctcgtgtgcggcgtcaggctttaccttctcctcgtactccatgaactgggtcagacaggcaccgggaaagggactggaatgggtgtcctccatctcgtcctcctcctcctacatctactacgccgatagcgtgaagggccggttcaccatttcgcgcgacaacgccaagaacaccctgtacctccaaatgaattcgctgcgggccgaagataccgctgtctattactgcgcccgcgacaactggggctcgctggactattggggccagggaaccctcgtcaccgtgtcaagcggagggggtggatccggaggcggaggatccggtggagggggaagcgacattcagatgactcagagcccgtcctccctgtctgcctccgtgggggatcgcgtgaccatcacatgccaggcctcacaagacatcagcaattacctgaactggtaccagcagaagcctggaaaggcccccaagctgctgatctacgatgccagcaacctggagactggggtgccttcaaggttctccggttccggaagcggcactgacttcaccttcactatctcgagcctgcaacccgaggacattgccacctactactgccagcagtacgacaaccttccgcacatgtacacgttcggccagggcaccaagctcgaaatcaaagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 130 - protein, 489 aa, synthetic construct
MLLLVTSLLLCELPHPAFLLIPEVQLVQSGGGVVQPGRSLRLSCAASGFTFSSYSMNWVRQAPGKGLEWVSSISSSSSYIYYADSVKGRFTISRDNAKNTLYLQMNSLRAEDTAVYYCARDNWGSLDYWGQGTLVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQQYDNLPHMYTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - DNA, 2293 bp, synthetic construct
  source 1..2293: mol_type = other DNA
atgctcttgctcgtgacttctttgcttttgtgcgaacttccgcacccagccttccttttgatacctcaggtacagcttcaacaaagcggaccgggacttgttaagcattcccaaaccctttctctcacgtgtgcaattagcggcgatagtgtatcctctaattctgcggcctggaactggatacgacaatcaccaagccggggactcgagtggttgggccgaacctactatcggtccaaatggtataatgactacgcagtatccgtgaaatctcgcattacgatcaatccagacacctccaaaaatcaattttctctgcagttgaatagcgtgactcccgaggacacggccgtttactattgcgcccaggaagttgaaccccacgatgcatttgatatttggggccagggaaccatggtgacagtgagtagtgggggtggaggatctggaggaggcggtagcggcgggggcggcagtgatatccagatgacgcagtcaccttccagcgtgtatgcgagtgtgggggacaaggtcaccataacctgtcgcgctagccaagatgtcagcgggtggctggcttggtaccagcagaaaccaggtttggctcctcagcttttgatctcaggagcgagcacgcttcagggtgaggtcccaagtcgctttagtggctctggctccgggacagacttcacgttgacgatcagcagtttgcagcctgaggatttcgcgacctactactgccagcaagcgaaatattttccgtacactttcggtcaggggaccaaattggagatcaaaggtgggggtggttcaggcggcggaggctcaggcggcggcggtagcggaggaggcggaagcgggggtggcggatcagaagtgcaactcgttcagagtggcgcggaggttaagaaacccggtgcatctgtaaaggttagctgtaaggcatcaggatacacttttaccagctattacatgcattgggtgagacaggctcccggtcaggggctcgaatggatggggttgatcaacccgagtggtggttcaacatcttacgcccagaagtttcagggccgagtaacaatgactcgggacacgtctacctcaactgtgtatatggagctttccagcctgcgctcagaggatacagcagtctattactgcgcacggtcagacagaggtataacggccactgatgcgttcgatatctggggacaagggactatggtaactgtgtcttccggaggaggaggtagtggagggggaggaagcggtggggggggctcacagtccgttttgactcagccaccaagcgtctcagtcgcaccggggcgaatggcgaaaattacttgcggcgggagcgacataggcaacaagaatgtgcattggtaccaacagaaaccaggtcaagcacctgttctcgtggtgtatgatgactacgatcgcccaagcgggatcccggagcggttctctggatcaaattctggtgatgcagccactctgacaatatcaacggtggaagtcggtgacgaggctgattacttctgccaagtatgggatggcagcggagatccctactggatgtttggaggaggtactcaactgacagttctgggcgcggccgcaacgaccactcctgcaccccgccctccgactccggccccaaccattgccagccagcccctgtccctgcggccggaagcctgcagaccggctgccggcggagccgtccatacccggggactggatttcgcctgcgatatctatatctgggcaccactcgccggaacctgtggagtgctgctgctgtcccttgtgatcaccctgtactgcaagcgcggacggaagaaactcttgtacatcttcaagcagccgttcatgcgccctgtgcaaaccacccaagaagaggacgggtgctcctgccggttcccggaagaggaagagggcggctgcgaactgcgcgtgaagttttcccggtccgccgacgctccggcgtaccagcaggggcaaaaccagctgtacaacgaacttaacctcggtcgccgggaagaatatgacgtgctggacaagcggcggggaagagatcccgagatgggtggaaagccgcggcggaagaaccctcaggagggcttgtacaacgagctgcaaaaggacaaaatggccgaagcctactccgagattggcatgaagggagagcgcagacgcgggaagggacacgatggactgtaccagggactgtcaaccgcgactaaggacacttacgacgccctgcacatgcaggccctgcccccgcgct

SEQ ID NO: 132 - protein, 764 aa, synthetic construct
MLLLVTSLLLCELPHPAFLLIPQVQLQQSGPGLVKHSQTLSLTCAISGDSVSSNSAAWNWIRQSPSRGLEWLGRTYYRSKWYNDYAVSVKSRITINPDTSKNQFSLQLNSVTPEDTAVYYCAQEVEPHDAFDIWGQGTMVTVSSGGGGSGGGGSGGGGSDIQMTQSPSSVYASVGDKVTITCRASQDVSGWLAWYQQKPGLAPQLLISGASTLQGEVPSRFSGSGSGTDFTLTISSLQPEDFATYYCQQAKYFPYTFGQGTKLEIKGGGGSGGGGSGGGGSGGGGSGGGGSEVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - DNA, 1476 bp, synthetic construct
  source 1..1476: mol_type = other DNA
atgcttcttttggtgacttcccttttgctgtgcgagttgccacaccccgccttcctgcttattccccaggtacagcttcaacagagtgggccgggactggtgaaacactcccaaacactttctctgacgtgcgctatatcaggtgactctgtttcatctaattctgctgcgtggaactggattcgacaatctcccagtcgcgggttggaatggctgggacgaacatattatcggtctaagtggtataacgattatgctgtatctgttaaatctcgaattacgattaatcctgacacctccaagaaccagttctccctccagttgaactcagtcacaccggaagacactgcggtctactattgcgctcaagaagtcgagccacatgatgcattcgacatctggggccagggaacgatggtcaccgtcagcagtggcggcggcggatctgggggtggcggttctggcggtggaggatcagacatacaaatgacgcagagtccctcaagtgtgtacgcgagtgtgggggataaggtaactattacgtgcagagcgtcacaggatgttagtggatggcttgcctggtatcagcagaagccaggccttgctccacagctccttatcagtggtgcttctacacttcagggcgaggttccgagtagattctctggttctggatctggtactgacttcactcttacaatttcttctttgcaaccagaagactttgcgacttattactgccaacaggccaaatacttcccttatacatttggccaaggtaccaagttggagataaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 134 - protein, 492 aa, synthetic construct
MLLLVTSLLLCELPHPAFLLIPQVQLQQSGPGLVKHSQTLSLTCAISGDSVSSNSAAWNWIRQSPSRGLEWLGRTYYRSKWYNDYAVSVKSRITINPDTSKNQFSLQLNSVTPEDTAVYYCAQEVEPHDAFDIWGQGTMVTVSSGGGGSGGGGSGGGGSDIQMTQSPSSVYASVGDKVTITCRASQDVSGWLAWYQQKPGLAPQLLISGASTLQGEVPSRFSGSGSGTDFTLTISSLQPEDFATYYCQQAKYFPYTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - DNA, 1476 bp, synthetic construct
  source 1..1476: mol_type = other DNA
atgttgctgctcgtgacctcgctccttctgtgcgagctgccccatccggcttttctgctcatccctcaagtgcagctgcagcagtccggtcctggactggtcaagccgtcccagactctgagcctgacttgcgccattagcgggaactcagtctcgtccaattcggcggcctggaactggatccggcagtcaccatcaaggggcctggaatggctcgggcgcacttactaccggtccaaatggtataacgactacgccgtgtccgtgaagtcccggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatctttggcgccagcactcttcagggggaggtgccatcacgcttctccggaggtggttccggcaccgacttcaccctgaccatcagcagcctccagcctgaggacttcgccacttactactgccaacaggccaagtacttcccctataccttcggacaaggcactaagctggaaatcaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 136 - protein, 492 aa, synthetic construct
MLLLVTSLLLCELPHPAFLLIPQVQLQQSGPGLVKPSQTLSLTCAISGNSVSSNSAAWNWIRQSPSRGLEWLGRTYYRSKWYNDYAVSVKSRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGGGSGTDFTLTISSLQPEDFATYYCQQAKYFPYTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - DNA, 190 bp, synthetic construct
  source 1..190: mol_type = other DNA
ggtacctgagctcagttctgagaaaagtagttctgagaaaagtagttctgagaaaagtagttctgagaaaagtagttctgagaaaagtctcgaggatatcaagatctggcctcggcggccaagcttagacactagagggtatataatggaagctcgacttccagcttggcaatccggtactgttggtaaa

SEQ ID NO: 138 - DNA, 214 bp, synthetic construct
  source 1..214: mol_type = other DNA
tgagctctgagtcagtgactcagtgagtcagtgactcagtgagtcagtgactcagctcgaggatctcgctagcgggaatttccggggactttccgggaatttccggggactttccgggaatttccagatctggcctcggcggccaagcttagacactagagggtatataatggaagctcgacttccagcttggcaatccggtactgttggtaaa

SEQ ID NO: 139 - DNA, 1670 bp, synthetic construct
  source 1..1670: mol_type = other DNA
ggtacctgagctcagttctgagaaaagtagttctgagaaaagtagttctgagaaaagtagttctgagaaaagtagttctgagaaaagtctcgaggatatcaagatctggcctcggcggccaagcttagacactagagggtatataatggaagctcgacttccagcttggcaatccggtactgttggtaaaggcgcgccccaccatgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgcgcccgcccctcggcccccgactcctgccccgacgatcgcttcccaacctctctcgctgcgcccggaagcatgccggcccgccgccggtggcgctgtccacactcgcggactggactttgataccgcactggcggccgtgatctgtagcgccctggccaccgtgctgctggcgctgctcatcctttgcgtgatctactgcaagcggcagcctaggcgaaagaagctcctctacattttcaagcaacccttcatgcgccccgtgcaaaccacccaggaggaggatggatgctcatgccggttccctgaggaagaagagggcggttgcgagctcagagtgaaattcagccggtcggctgacgccccggcgtaccagcagggccagaaccagctgtacaatgagctcaacctggggcgccgcgaagagtacgacgtgctggacaagaggagaggcagagatccggaaatgggcggaaagccaaggcggaagaacccgcaggaaggtctttacaacgaactgcagaaggacaagatggccgaggcctactccgagattgggatgaagggagaaagacggaggggaaagggacatgacggactttaccagggcctgagcactgccacgaaggacacctatgatgccctgcacatgcaggcgctgccgcctcgg

SEQ ID NO: 140 - DNA, 1694 bp, synthetic construct
  source 1..1694: mol_type = other DNA
tgagctctgagtcagtgactcagtgagtcagtgactcagtgagtcagtgactcagctcgaggatctcgctagcgggaatttccggggactttccgggaatttccggggactttccgggaatttccagatctggcctcggcggccaagcttagacactagagggtatataatggaagctcgacttccagcttggcaatccggtactgttggtaaaggcgcgccccaccatgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgcgcccgcccctcggcccccgactcctgccccgacgatcgcttcccaacctctctcgctgcgcccggaagcatgccggcccgccgccggtggcgctgtccacactcgcggactggactttgataccgcactggcggccgtgatctgtagcgccctggccaccgtgctgctggcgctgctcatcctttgcgtgatctactgcaagcggcagcctaggcgaaagaagctcctctacattttcaagcaacccttcatgcgccccgtgcaaaccacccaggaggaggatggatgctcatgccggttccctgaggaagaagagggcggttgcgagctcagagtgaaattcagccggtcggctgacgccccggcgtaccagcagggccagaaccagctgtacaatgagctcaacctggggcgccgcgaagagtacgacgtgctggacaagaggagaggcagagatccggaaatgggcggaaagccaaggcggaagaacccgcaggaaggtctttacaacgaactgcagaaggacaagatggccgaggcctactccgagattgggatgaagggagaaagacggaggggaaagggacatgacggactttaccagggcctgagcactgccacgaaggacacctatgatgccctgcacatgcaggcgctgccgcctcgg

SEQ ID NO: 141 - DNA, 735 bp, synthetic construct
  source 1..735: mol_type = other DNA
gacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatcc

SEQ ID NO: 142 - DNA, 1473 bp, synthetic construct
  source 1..1473: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 143 - protein, 491 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - DNA, 750 bp, synthetic construct
  source 1..750: mol_type = other DNA
gaagtgcaattggtgcagagcggtggaggacttgtgaaacctggtggatccctgagactttcctgtgccgcttcgggcttcaccttctcggactactacatgtcctggattcgccaggcccctggaaagggactggaatgggtgtcatacatcagctcctccggttccaccatctactatgccgattccgtgaagggcagattcaccatctcgcgcgacaacgccaagaacactctctatctgcaaatgaactcactgcgggctgaggacaccgcggtctactactgcgcccgggacctcagcggaaagtccagcggatggtcccattacttcgattactggggacagggaaccctggtcaccgtgtccagcggcggggggggctcgggtggcggcggctccggcggcggcgggagcaacttcatgctgactcagccccactccgtgtccgagagcccgggaaagaccgtgactatttcgtgcacacggtcctccgggagcattgcgaacaactacgtgcagtggtaccagcagcggcccgatagggccccaaccactgtgatctacgaagatgaccagcggccgtctggagtcccggaccgcttctcggggtccatcgactcatcatccaattccgcatcgctgacgatcagcggactgaagatcgaggacgaagccgattactactgccagtcctacgacggcaccaactgggtctttgggggtggaaccaagctgactgtgctcgga

SEQ ID NO: 145 - protein, 250 aa, synthetic construct
EVQLVQSGGGLVKPGGSLRLSCAASGFTFSDYYMSWIRQAPGKGLEWVSYISSSGSTIYYADSVKGRFTISRDNAKNTLYLQMNSLRAEDTAVYYCARDLSGKSSGWSHYFDYWGQGTLVTVSSGGGGSGGGGSGGGGSNFMLTQPHSVSESPGKTVTISCTRSSGSIANNYVQWYQQRPDRAPTTVIYEDDQRPSGVPDRFSGSIDSSSNSASLTISGLKIEDEADYYCQSYDGTNWVFGGGTKLTVLG

SEQ ID NO: 146 - DNA, 729 bp, synthetic construct
  source 1..729: mol_type = other DNA
gaagtgcaacttgtcgaaagcggtggaggtcttgtccaacctggtcgctccctgaggctctcgtgtgccgcgagcggattcaccttctcatcgtacgctatgtcctgggtcagacaggctcctggaaagggcctggaatgggtggccgtgatctcctacgacggcagcaacaagtattacgccgactcagtgaagggccggttcaccatttcccgggacaacagcaagaacaccctgtacttgcaaatgaactccctgcgggccgaggataccgcggtgtactactgcgcccacctccgctttggatacggaatggatgtctggggacagggaactaccgtgaccgtgtcgtccgggggggggggaagcggcggcgggggatcgggtggcggcggatcccagactgtggtcacccaagagccttcactgaccgtgtccccgggtggcaccgtgacgctgacttgcgcgtcatctaccggggccgtgacctcggaccactacccctgctggttccagcagaaacccggacatccaccgagagccctggtgtactccactgacaccatccactcctggactccggcccggttctccggaagcctcctgggcgggaaggccgcactgacagtgtccggagtgcagcccgaggatgaagccgactactactgtctgctctactatgggggagcacgcgtgttcggtggcggcactcagctgaccgtgctggga

SEQ ID NO: 147 - protein, 243 aa, synthetic construct
EVQLVESGGGLVQPGRSLRLSCAASGFTFSSYAMSWVRQAPGKGLEWVAVISYDGSNKYYADSVKGRFTISRDNSKNTLYLQMNSLRAEDTAVYYCAHLRFGYGMDVWGQGTTVTVSSGGGGSGGGGSGGGGSQTVVTQEPSLTVSPGGTVTLTCASSTGAVTSDHYPCWFQQKPGHPPRALVYSTDTIHSWTPARFSGSLLGGKAALTVSGVQPEDEADYYCLLYYGGARVFGGGTQLTVLG

SEQ ID NO: 148 - DNA, 723 bp, synthetic construct
  source 1..723: mol_type = other DNA
caagtccaactcgtccagtccggtgccgaagtcaagaagcctggctcatccgtgaaagtgtcctgcaaagcatcgggcggaaccttctcctcctatgccatttcctgggtccgccaggcaccgggccagggtctggagtggatgggcgggattatccctatcttcggaactgccaaccacgcccaaaagttccagggacgcgtgaccattaccgccgatgaatcaacctcaaccgcctacatggaactgtccagcttgaggtccgaggacaccgccgtgtactactgcgcgttcatgatggtgccggagtactactttgactactggggccagggaacccttgtgaccgtgtcgtccggtggtggcggatccgggggggggggatctgggggcggcggaagcgatatccagatgacccagtcgccatcgagcctgtccgcttccgtgggcgacagagtgacgatcacttgccgggcttcacaaggcatcagaaatgacctgggctggtatcagcagaagcccggagaagcgcccaagcggctgatctacgcggccagcaccctgcaaaacggagtgccttcgcggttctccgggagcggctccggaactgacttcactctgactattaacagcctccagcccgaggatttcgccacatactactgtcagcagtacaacagctacccgtacaccttcggacagggaactaagctcgaaatcaag

SEQ ID NO: 149 - protein, 241 aa, synthetic construct
QVQLVQSGAEVKKPGSSVKVSCKASGGTFSSYAISWVRQAPGQGLEWMGGIIPIFGTANHAQKFQGRVTITADESTSTAYMELSSLRSEDTAVYYCAFMMVPEYYFDYWGQGTLVTVSSGGGGSGGGGSGGGGSDIQMTQSPSSLSASVGDRVTITCRASQGIRNDLGWYQQKPGEAPKRLIYAASTLQNGVPSRFSGSGSGTDFTLTINSLQPEDFATYYCQQYNSYPYTFGQGTKLEIK

SEQ ID NO: 150 - DNA, 756 bp, synthetic construct
  source 1..756: mol_type = other DNA
caactccaactccaagaatctggaccaggcctcgtgaagccctcccaaactctgtccctgacctgtaccgtgtcgggtggaagcatttcgagcggtggatactactggtcctggatcaggcagcatcctggaaagggactggagtggattgggtacatctactactccggctcaacctactacaacccgtccttgaaatcgcgcgtgacgatctccgtggacacttcaaagaaccagttcagcctgaagctttcctccgtgaccgcggccgatacagcggtgtactactgcgctcgggatcagagcgtggccgaccctggtggcggctactactactacggaatggatgtctggggacagggaaccaccgtgactgtgtccagcgggggcggcggatccgggggggggggatcgggcggcggcggttcgcagtccgtgctgacccagccacctagcgtgtcagtggcaccgggacagaccgcctccatttcctgcgggggaaatgacttcggtagccgctccgtgtcatggtatcaccagaagccgggacaggccccggtgctggtcatctatgacgacaacgacagaccctcgggcatccccgaacggttttcgggaagcacctccggagacactgccaccctgaccatctcccgggtcgaggtcggcgatgaagccgattactactgccaagtctgggacgacgactccgaccactgggtgttcggcggcggaactaagctgactgtgctgggg

SEQ ID NO: 151 - protein, 252 aa, synthetic construct
QLQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDQSVADPGGGYYYYGMDVWGQGTTVTVSSGGGGSGGGGSGGGGSQSVLTQPPSVSVAPGQTASISCGGNDFGSRSVSWYHQKPGQAPVLVIYDDNDRPSGIPERFSGSTSGDTATLTISRVEVGDEADYYCQVWDDDSDHWVFGGGTKLTVLG

SEQ ID NO: 152 - DNA, 723 bp, synthetic construct
  source 1..723: mol_type = other DNA
caggtgcagctgcaggagtccggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatacccgtagacacgtccaagaaccagttctccctgaagctgagctctgtgaccgccgcagacacggctgtgtattactgtgcgagacacctggggggtgatgcttttgatatctggggccaagggaccacggtcaccgtctcctcaggaggtggcgggtctggtggtggcggtagcggtggaggcggatccctgcctgtgctgactcagcccccctcggtgtcagtggccccaggacagacggccaggattacctgtggggggaacaacattggaagtaaaagtgtgcactggtaccagcagaagccaggccaggcccctgtgctggtcgtctatgatgatagcgaccggccctcagggatccctgagcgattctctggctccaactctgggaacacagccactctgaccatcagcgggacccaggctatggatgaggctgactacttctgtcagtcttatgatagcagcaatcccgtggtattcggcggagggacccagctcaccgtttta

SEQ ID NO: 153 - protein, 241 aa, synthetic construct
QVQLQESGPGLVKPSETLSLTCTVSGGSISSSSYYWGWIRQPPGKGLEWIGSIYYSGSTYYNPSLKSRVTIPVDTSKNQFSLKLSSVTAADTAVYYCARHLGGDAFDIWGQGTTVTVSSGGGGSGGGGSGGGGSLPVLTQPPSVSVAPGQTARITCGGNNIGSKSVHWYQQKPGQAPVLVVYDDSDRPSGIPERFSGSNSGNTATLTISGTQAMDEADYFCQSYDSSNPVVFGGGTQLTVL

SEQ ID NO: 154 - DNA, 732 bp, synthetic construct
  source 1..732: mol_type = other DNA
caggcggcccaggtacagctgcagcagtcaggggctgaggtgaagaagcctgggtcctcggtgaaggtctcctgcaaggcttctggaggcaccttcagcagctatgctatcagctgggtgcgacaggcccctggacaagggcttgagtggatgggatggatcaaccctaacagtggtggcacaaactatgcacagaggtttcagggcagggtcaccatgaccagggacacgtccatcagcacagcctacatggagctgagcaggctgagatctgacgacacggccgtgtattactgtgcgagttataatgatgcttttgatatctggggccaaggcaccctggtcaccgtctcctcaggaggtggcgggtctggtggtggcggtagcggtggtggcggatccaattttatgctgactcagccccactctgtgtcggagtctccggggaagacggtaaccatctcctgcacccgcagcagtggcagcattgccagcaactatgtgcagtggtaccagcagcgcccgggcagtgcccccaccattgtgatctatgaggatgatcaaagaccctctggggtccctgatcggttctctggctccatcgacacctcctccaactctgcctccctcaccatctctggactgcagagtgaggacgaggctgactactactgtcagtcttatgagcccggcaatggggtattcggcggagggaccaaggtcaccgtccta

SEQ ID NO: 155 - protein, 244 aa, synthetic construct
QAAQVQLQQSGAEVKKPGSSVKVSCKASGGTFSSYAISWVRQAPGQGLEWMGWINPNSGGTNYAQRFQGRVTMTRDTSISTAYMELSRLRSDDTAVYYCASYNDAFDIWGQGTLVTVSSGGGGSGGGGSGGGGSNFMLTQPHSVSESPGKTVTISCTRSSGSIASNYVQWYQQRPGSAPTIVIYEDDQRPSGVPDRFSGSIDTSSNSASLTISGLQSEDEADYYCQSYEPGNGVFGGGTKVTVL

SEQ ID NO: 156 - DNA, 744 bp, synthetic construct
  source 1..744: mol_type = other DNA
caagaacagcttgtagagtccggcggtagattggtgacaccgggggggagccttaccctgtcttgtaaggcatctgggttcgatttcagtgcgtattatatgagctgggttcggcaggcgcccgggaaggggctggaatggatagccactatatacccgtcatccggcaagacttactacgcgacttgggtaaacgggaggtttacgataagctcagataacgcccaaaacacggttgatctccaaatgaatagcttgaccgccgctgatagggcgacctatttctgtgcgcgggactcttacgctgatgacggggccctcttcaatatatggggaccgggaacgctcgtaaccatatcatctggaggaggtgggagcggaggcggagggtcaggtgggggcgggagcgaactcgtacttacacaatctccaagcgtaagcgcagcgttggggagtccagcaaagatcacctgcactttgtcaagcgcccacaaaacggatacgatagattggtatcagcaactccaaggtgaagcgccacgatatctcatgcaggtacagagcgacgggagttatactaagaggcccggggtcccagacagattcagtggcagcagttcaggtgccgacagatacctgataataccctcagttcaagccgatgatgaagccgattactactgtggggctgactacataggtgggtatgttttcgggggcggcactcaattgacagttacaggg

SEQ ID NO: 157 - protein, 248 aa, synthetic construct
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 158 - DNA, 720 bp, synthetic construct
  source 1..720: mol_type = other DNA
gaagtgcaactcgtcgaaactggagccgaagtgaaaaagcctggagcgtccgtcaaagtgtcgtgcaaggcctccggctacaccttcacgacctactacgtgcactgggtcagacaggctccgggtcaagggctggagtggatgggcatcattaacccctccggtggaagcacctcctatgcgcaaaagttccagggtcgcgtcaccatgactcgcgatacctccacttccactgtgtacatggaactgagctccctgaggtccgaggacaccgccgtgtactactgcgcacgggatggaggcttgggcggctacgaggcttggggacagggcaccctcgtgactgtgtcaagcggagggggtggatccggagggggaggatcaggcggtggtggaagcgatatccagcttacccagtcgccttccgcgctgtctgcatcggccggcgacagagtgacaattacctgtcaagccagccaggacatctccaactatctgaactggtaccagcagaagcccggaaaggctccgaagctgctgatctacgacgccagcaacctggaacggggcgtgccatcacggttctcgggatcagggtcgggcactgagttcaccttcaccatctcctccctccaacccgaggacattgccacctactactgccagcagtacgacaacctcccgatcacctttggacaggggactcgcctggaaatcaag

SEQ ID NO: 159 - protein, 240 aa, synthetic construct
EVQLVETGAEVKKPGASVKVSCKASGYTFTTYYVHWVRQAPGQGLEWMGIINPSGGSTSYAQKFQGRVTMTRDTSTSTVYMELSSLRSEDTAVYYCARDGGLGGYEAWGQGTLVTVSSGGGGSGGGGSGGGGSDIQLTQSPSALSASAGDRVTITCQASQDISNYLNWYQQKPGKAPKLLIYDASNLERGVPSRFSGSGSGTEFTFTISSLQPEDIATYYCQQYDNLPITFGQGTRLEIK

SEQ ID NO: 160 - DNA, 759 bp, synthetic construct
  source 1..759: mol_type = other DNA
gaagtgcaattggtccagagcggaggaggacttgtgaagccaggcggatccctgagattgtcatgcgccgcatcggggttcaccttttcctcctactccatgaactgggtcagacaggcgcccggaaagggacttgaatgggtgtcgtccatttcctcctcctcgtcctacatctactacgccgactccgtgaagggccgcttcaccatctcccgggacaacgccaagaacagcctgtatctccaaatgaactccctgcgggccgaagatactgctgtgtattactgcgctcgggacttcccgtacgactcatcgggctattactcggacgcgttcgatatctggggccagggaactatggtcaccgtcagctctggtggcggtggttccggagggggtggatccggtggcggaggatcagagattgtgctgacccagtccccgctgtcactgcccgtgactccgggagagcctgcctcgatctcgtgtcggtccagccagtccctgctgcactcgaatgggtacaactacctcgattggtacctccaaaagcctgggcagtcaccccaactgctgatctacctcgggagcaacagagccagcggagtgcctgaccgctttagcggttccggatccggcaccgacttcaccctgaaaatcagccgggtggaagccgaggatgtcggcgtgtactactgcatgcaggcactgcagactctggggtacaccttcggccagggcacgaagctcgagatcaag

SEQ ID NO: 161 - protein, 253 aa, synthetic construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDFPYDSSGYYSDAFDIWGQGTMVTVSSGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTLGYTFGQGTKLEIK

SEQ ID NO: 162 - DNA, 747 bp, synthetic construct
  source 1..747: mol_type = other DNA
caagtgcaactcgtccaatccggtgccgaagtcaagaagcctggttcctccgtgaaagtgtcgtgcaaagccagcggcgggacttttagctcctacgcgatcagctgggtcagacaggcccctggacaaggcctcgagtggatgggcggcatcattccgattttcggtaccgccaactacgcccagaagttccagggacgcgtgaccattactaccgacgagagcacctcaaccgcatacatggaactgtccagcctgcgctccgaggacacggctgtgtactattgcgccagacggggatggggaggattctcctccggctccgcattcgacatctggggacagggcactatggtcactgtgtcatccgggggaggaggatcaggcggtggaggatccggtggtggcggatccaacttcatgctgacccagccccactcagtgtcggaatcgcccggcaacaccgtgactatcagctgcaccggatccagcgggaccatcggctctaatttcgtgcagtggtaccagcagtccccagggagagctccgaccctgttgatctacgaggacacaaagcggccaagcggagtgccgcctagattcgccggctccgtggattcctcgtccaactcggcgtcgctgaccatcagcggactcaagactgaagatgaagccgactactactgtcagtcctacgactcgagcaactgggtgtttgggggcgggactaagctgaccgtgcttgga

SEQ ID NO: 163 - protein, 249 aa, synthetic construct
QVQLVQSGAEVKKPGSSVKVSCKASGGTFSSYAISWVRQAPGQGLEWMGGIIPIFGTANYAQKFQGRVTITTDESTSTAYMELSSLRSEDTAVYYCARRGWGGFSSGSAFDIWGQGTMVTVSSGGGGSGGGGSGGGGSNFMLTQPHSVSESPGNTVTISCTGSSGTIGSNFVQWYQQSPGRAPTLLIYEDTKRPSGVPPRFAGSVDSSSNSASLTISGLKTEDEADYYCQSYDSSNWVFGGGTKLTVLG

SEQ ID NO: 164 - DNA, 735 bp, synthetic construct
  source 1..735: mol_type = other DNA
gaagtccaattggtgcagagcggatccgaacttaagaaacctggcgcgagcgtgaaagtgtcctgcaaggcctccggagggactttctcgtcgtacgccattagctgggtccgccaagctcctggccaaggcctggagtggatgggcgggattatccccatcttcgggactgcgaactacgcccagaagtttcagggccgggtcactatcaccgccgacgaatcaacctcgaccgcctacatggaactgtcctcgcttcggtccgaggatactgccgtgtactattgtgcctcaacggccagacgcggatgggacaccgctggtccgctcgattactggggccagggaaccctcgtgaccgtcagctccggaggaggaggctccggtggtggaggatccgggggtggtggatccgacatccaaatgacccagtccccctcgtccctgagcgcctctgtgggcgacagagtgacaattgcatgcagggcctcacagactatctcccgctacctgaactggtaccagcagaagccaggaaaggcccctaagctgctcatctacgctgcgtcctcgctccaatccggggtgtcctcacggttttccggatcgggttccggcaccgagttcaccctgaccatcagcagcctgcagcccgaggacttcgcaacctacttctgccagcaaacctactccccgccgattacgttcggacaggggactcggctggaaatcaag

SEQ ID NO: 165 - protein, 245 aa, synthetic construct
EVQLVQSGSELKKPGASVKVSCKASGGTFSSYAISWVRQAPGQGLEWMGGIIPIFGTANYAQKFQGRVTITADESTSTAYMELSSLRSEDTAVYYCASTARRGWDTAGPLDYWGQGTLVTVSSGGGGSGGGGSGGGGSDIQMTQSPSSLSASVGDRVTIACRASQTISRYLNWYQQKPGKAPKLLIYAASSLQSGVSSRFSGSGSGTEFTLTISSLQPEDFATYFCQQTYSPPITFGQGTRLEIK

SEQ ID NO: 166 - DNA, 729 bp, synthetic construct
  source 1..729: mol_type = other DNA
gaagtgcaactcgtcgaaacaggggcagaagtgaaaaacccaggctcaagcgtgaaagtgtcgtgcaaggcttcgggcggaactctgtccaactacgccatctcctgggtccgccaagctccgggaaagggcctcgagtggatgggcggaatcattcccattttcgggaccgccaactacgcgcaaaagttccagggccgggtcactatcaccgcggacgaaagcaccagcaccgcctacatggaactgtcctccctgcgctccgaggacactgccgtgtactattgcgcccggaggtcatcgtggtaccccgagggctgcttccagcactggggacagggcactctcgtgaccgtgtcgtcgggtggtggtggatcaggagggggaggatccggaggaggcggaagcgatattcagctgacccagtcaccgagctccctgtccgcctccaccggagacagagtgaccatcacgtgtcgggcctcccaagggatctcctcctacctggcctggtaccagcagaagcctggaaaggcaccgaagttgctgatctacgccgcgagcacccttcagtccggagtgcctagccgcttctcgggttccggctctggcactgacttcactctgaccattagctgcctgcagtccgaggattttgccacctactactgccagcagtactatagctaccccctgaccttcgggggcggaaccaagctcgacatcaag

SEQ ID NO: 167 - protein, 243 aa, synthetic construct
EVQLVETGAEVKNPGSSVKVSCKASGGTLSNYAISWVRQAPGKGLEWMGGIIPIFGTANYAQKFQGRVTITADESTSTAYMELSSLRSEDTAVYYCARRSSWYPEGCFQHWGQGTLVTVSSGGGGSGGGGSGGGGSDIQLTQSPSSLSASTGDRVTITCRASQGISSYLAWYQQKPGKAPKLLIYAASTLQSGVPSRFSGSGSGTDFTLTISCLQSEDFATYYCQQYYSYPLTFGGGTKLDIK

SEQ ID NO: 168 - DNA, 732 bp, synthetic construct
  source 1..732: mol_type = other DNA
gaagtgcaactcgtccaatctggtgccgaagtcaagaagcctggctcaagcgtgaaagtgtcctgcaaagcgtcgggagggaccttcagctcctacgccatttcctgggtccgccaagcaccaggacagggcctggagtggatgggcggcatcatcccgatcttcgggactgccaactacgcccagaagttccaggggagagtgaccattaccgccgacgagtcgaccagcacggcctacatggaactgtccagcctgcgctccgaggacactgccgtgtactactgcgcgagggccagactcggtggagcgttcgacatctggggacagggcaccatggtcaccgtgtcatccggtggcggaggatccggtggtggcggatcaggagggggaggatcccagtccgtgctgactcagcctccctccgtgagcgctgcaccgggacagaaggtcaccatctcatgctcggggggaagctccaacatcgggaaccactacgtgtcctggtaccaacagttgcctggtgccgctccaaagctgctgatctatgacgataacaagcggccgtccggaatccccgaccggttctcggggtctagatccggaaccagcgcaactctcggcattaccggactgcagagcggcgatgaggccgactactactgtggcacatgggactcgtcgctggctgcccacgtgtttggcactggcaccaaggtcaccgtgcttgga

SEQ ID NO: 169 - protein, 244 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARARLGGAFDIWGQGTMVTVSSGGGGSGGGGSGGGGSQSVLTQPPSVSAAPGQKVTISCSGGSSNIGNHYVSWYQQLPGAAPKLLIYDDNKRPSGIPDRFSGSRSGTSATLGITGLQSGDEADYYCGTWDSSLAAHVFGTGTKVTVLG

SEQ ID NO: 170 - DNA, 747 bp, synthetic construct
  source 1..747: mol_type = other DNA
gaagtgcaacttgtccagagcggagccgaagtgaagaaacctggatcctccgtcaaagtgtcgtgcaaggcttcgggcggaaccttctcctcgtacgcgatctcatgggtcagacaggcacccggacagggactggagtggatgggcggcatcattcccatcttcggcaccgctaattacgcccagaagtttcaggggagagtgaccatcaccgccgacgagtccacctccactgcctacatggaactgtcctcactgaggtccgaggatactgccgtgtactactgcgcgtcgcaaaaggggggtggatggtccattgacgccttcgatatttggggacaggggacgatggtcacagtgtcatccggcggtggtggatccggtggtggcggatccggaggaggaggcagccagtccgtgctgacccagccgcctagcgtgtcggccgcatctgggcagcgcgtgaccatttcctgttccgggtcctcgtccaacatcggcaacaactacgcctcctggtaccaacagctcccgggaatggcccctaagctgctgatctacgaggacaacaagcggccatccgggatctcagaccggttcagcggatcccagtccggcacttccgcgagcctcgccatcaccggactgcaggctgaggacgaagccgactactactgccaatcatatgacagctcgctcagcggcgatgtggtgttcggcggtggcactaagctgaccgtgttggga

SEQ ID NO: 171 - protein, 249 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCASQKGGGWSIDAFDIWGQGTMVTVSSGGGGSGGGGSGGGGSQSVLTQPPSVSAASGQRVTISCSGSSSNIGNNYASWYQQLPGMAPKLLIYEDNKRPSGISDRFSGSQSGTSASLAITGLQAEDEADYYCQSYDSSLSGDVVFGGGTKLTVLG

SEQ ID NO: 172 - DNA, 735 bp, synthetic construct
  source 1..735: mol_type = other DNA
caagtccagttgcaacaatggggagcaggccttctgaaaccgtccgagacactgagcctgacgtgcgccgtctatggcggatcgttctccggatactactggtcgtggatcagacagcctccgggaaagggtctggaatggatcggcgaaatcaaccacagcggcagcaccaattacaacccgtcactgaagtcaagggtcaccattagcgtggacacttccaagaaccagttctccctgaaactgtcgagcgtgaccgctgccgatactgccgtgtactactgtgcccgcggccaagtcaagtatagctcaagcctcggctactggggccagggaaccctcgtgaccgtgtcctcgggtggaggaggctccggtggtggaggatccggtggcggaggatcgcagtccgtgctgacccagcctccctccgtgtctgctgcccctgggcaaaaggtcaccatttcgtgctccggctcatcgtccaacatcgggaacaactttgtgtcctggtaccagcagctgcccggtactgccccaaagctgctgatctacgaggacaacaagcgcccatccgggattccggatcggttcagcggatcacggtccggaactagcgcgaccctggggatcaccgggctccagactggcgacgaagcggactactactgcggaacttgggactcctccttgggggcctgggtgttcggcggagggaccaagctcaccgtgcttgga

SEQ ID NO: 173 - protein, 245 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGQVKYSSSLGYWGQGTLVTVSSGGGGSGGGGSGGGGSQSVLTQPPSVSAAPGQKVTISCSGSSSNIGNNFVSWYQQLPGTAPKLLIYEDNKRPSGIPDRFSGSRSGTSATLGITGLQTGDEADYYCGTWDSSLGAWVFGGGTKLTVLG

SEQ ID NO: 174 - DNA, 723 bp, synthetic construct
  source 1..723: mol_type = other DNA
caagtccaactcgtcgaaactggtggtggcctcgtgaagcctggaggatccctgcgcctttcctgtgccgcttccggctttactttctcgtcgtactccatgaactgggtcagacaggctcccggaaagggcctggaatgggtgtcctccatctcgtcctcatcctcctacatctattacgcggactccgtgaagggcagattcaccatttcccgggacaacgccaagaacagcttgtacctccaaatgaactccctgcgggcagaggacaccgccgtgtactactgcgcgagggatggggatttctggagcggagccatcgactactggggccagggaactctcgtgaccgtcagctccggtggtggtggaagcggaggcggaggttctggggggggaggatcagacattcagctgacccagtcgccatcctccctgagcgcctcagtgggggaccgcgtgactattacatgccaggcctcccaagatatctcgaactacctgaactggtatcagcagaagcctggaaaggccccgaagctgttgatctacgatgccagcaacctggagactggggtgccttcccggttctcgggatcaggctcgggcaccgatttcaccttcacgatcagcagcctgcagcccgaggacattgcaacctactactgccagcagtacgacaatctgccgctttttgggggaggcaccaagctggaaatcaaa

SEQ ID NO: 175 - protein, 241 aa, synthetic construct
QVQLVETGGGLVKPGGSLRLSCAASGFTFSSYSMNWVRQAPGKGLEWVSSISSSSSYIYYADSVKGRFTISRDNAKNSLYLQMNSLRAEDTAVYYCARDGDFWSGAIDYWGQGTLVTVSSGGGGSGGGGSGGGGSDIQLTQSPSSLSASVGDRVTITCQASQDISNYLNWYQQKPGKAPKLLIYDASNLETGVPSRFSGSGSGTDFTFTISSLQPEDIATYYCQQYDNLPLFGGGTKLEIK

SEQ ID NO: 176 - DNA, 723 bp, synthetic construct
  source 1..723: mol_type = other DNA
gaggtgcaattggtgcagtcaggtggtggagtggtgcagccaggaagatcccttagactctcgtgtgcggcgtcaggctttaccttctcctcgtactccatgaactgggtcagacaggcaccgggaaagggactggaatgggtgtcctccatctcgtcctcctcctcctacatctactacgccgatagcgtgaagggccggttcaccatttcgcgcgacaacgccaagaacaccctgtacctccaaatgaattcgctgcgggccgaagataccgctgtctattactgcgcccgcgacaactggggctcgctggactattggggccagggaaccctcgtcaccgtgtcaagcggagggggtggatccggaggcggaggatccggtggagggggaagcgacattcagatgactcagagcccgtcctccctgtctgcctccgtgggggatcgcgtgaccatcacatgccaggcctcacaagacatcagcaattacctgaactggtaccagcagaagcctggaaaggcccccaagctgctgatctacgatgccagcaacctggagactggggtgccttcaaggttctccggttccggaagcggcactgacttcaccttcactatctcgagcctgcaacccgaggacattgccacctactactgccagcagtacgacaaccttccgcacatgtacacgttcggccagggcaccaagctcgaaatcaaa

SEQ ID NO: 177 - protein, 241 aa, synthetic construct
EVQLVQSGGGVVQPGRSLRLSCAASGFTFSSYSMNWVRQAPGKGLEWVSSISSSSSYIYYADSVKGRFTISRDNAKNTLYLQMNSLRAEDTAVYYCARDNWGSLDYWGQGTLVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQQYDNLPHMYTFGQGTKLEIK

SEQ ID NO: 178 - DNA, 1548 bp, synthetic construct
  source 1..1548: mol_type = other DNA
caggtacagcttcaacaaagcggaccgggacttgttaagcattcccaaaccctttctctcacgtgtgcaattagcggcgatagtgtatcctctaattctgcggcctggaactggatacgacaatcaccaagccggggactcgagtggttgggccgaacctactatcggtccaaatggtataatgactacgcagtatccgtgaaatctcgcattacgatcaatccagacacctccaaaaatcaattttctctgcagttgaatagcgtgactcccgaggacacggccgtttactattgcgcccaggaagttgaaccccacgatgcatttgatatttggggccagggaaccatggtgacagtgagtagtgggggtggaggatctggaggaggcggtagcggcgggggcggcagtgatatccagatgacgcagtcaccttccagcgtgtatgcgagtgtgggggacaaggtcaccataacctgtcgcgctagccaagatgtcagcgggtggctggcttggtaccagcagaaaccaggtttggctcctcagcttttgatctcaggagcgagcacgcttcagggtgaggtcccaagtcgctttagtggctctggctccgggacagacttcacgttgacgatcagcagtttgcagcctgaggatttcgcgacctactactgccagcaagcgaaatattttccgtacactttcggtcaggggaccaaattggagatcaaaggtgggggtggttcaggcggcggaggctcaggcggcggcggtagcggaggaggcggaagcgggggtggcggatcagaagtgcaactcgttcagagtggcgcggaggttaagaaacccggtgcatctgtaaaggttagctgtaaggcatcaggatacacttttaccagctattacatgcattgggtgagacaggctcccggtcaggggctcgaatggatggggttgatcaacccgagtggtggttcaacatcttacgcccagaagtttcagggccgagtaacaatgactcgggacacgtctacctcaactgtgtatatggagctttccagcctgcgctcagaggatacagcagtctattactgcgcacggtcagacagaggtataacggccactgatgcgttcgatatctggggacaagggactatggtaactgtgtcttccggaggaggaggtagtggagggggaggaagcggtggggggggctcacagtccgttttgactcagccaccaagcgtctcagtcgcaccggggcgaatggcgaaaattacttgcggcgggagcgacataggcaacaagaatgtgcattggtaccaacagaaaccaggtcaagcacctgttctcgtggtgtatgatgactacgatcgcccaagcgggatcccggagcggttctctggatcaaattctggtgatgcagccactctgacaatatcaacggtggaagtcggtgacgaggctgattacttctgccaagtatgggatggcagcggagatccctactggatgtttggaggaggtactcaactgacagttctgggc

SEQ ID NO: 179 - protein, 516 aa, synthetic construct
QVQLQQSGPGLVKHSQTLSLTCAISGDSVSSNSAAWNWIRQSPSRGLEWLGRTYYRSKWYNDYAVSVKSRITINPDTSKNQFSLQLNSVTPEDTAVYYCAQEVEPHDAFDIWGQGTMVTVSSGGGGSGGGGSGGGGSDIQMTQSPSSVYASVGDKVTITCRASQDVSGWLAWYQQKPGLAPQLLISGASTLQGEVPSRFSGSGSGTDFTLTISSLQPEDFATYYCQQAKYFPYTFGQGTKLEIKGGGGSGGGGSGGGGSGGGGSGGGGSEVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLG

SEQ ID NO: 180 - DNA, 732 bp, synthetic construct
  source 1..732: mol_type = other DNA
caggtacagctgcagcagtcaggtccaggactggtgaagcactcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagttgaactctgtgactcccgaggacacggctgtgtattactgtgcccaagaggtagaacctcatgatgcttttgatatctggggccaagggacaatggtcaccgtctcttcaggaggtggcgggtctggcggtggaggtagcggtggtggcggatccgacatccagatgacccagtctccatcttccgtgtatgcatctgtaggagacaaagtcaccatcacttgtcgggcgagtcaggatgttagcggctggttagcctggtatcagcagaaaccagggctagcccctcagctcctgatctctggtgcatccactttgcaaggtgaagtcccatcaaggttcagcggcagtggatctgggacagattttactctcaccatcagcagcctgcagcctgaagattttgccacttattattgtcaacaggctaaatatttcccttacacttttggccaggggaccaagctggaaatcaaa

SEQ ID NO: 181 - protein, 244 aa, synthetic construct
QVQLQQSGPGLVKHSQTLSLTCAISGDSVSSNSAAWNWIRQSPSRGLEWLGRTYYRSKWYNDYAVSVKSRITINPDTSKNQFSLQLNSVTPEDTAVYYCAQEVEPHDAFDIWGQGTMVTVSSGGGGSGGGGSGGGGSDIQMTQSPSSVYASVGDKVTITCRASQDVSGWLAWYQQKPGLAPQLLISGASTLQGEVPSRFSGSGSGTDFTLTISSLQPEDFATYYCQQAKYFPYTFGQGTKLEIK

SEQ ID NO: 182 - DNA, 732 bp, synthetic construct
  source 1..732: mol_type = other DNA
caggtacagctgcagcagtcaggtccaggactggtgaagccctcgcagaccctctcactcacctgtgccatctcagggaacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcccaagaggtagaacctcaagatgcttttgatatctggggccaagggacaatggtcaccgtctcttcaggaggtggcgggtctggcggtggaggtagcggtggtggcggatccgacatccagatgacccagtctccatcttccgtgtctgcatctgtaggagacaaagtcaccatcacttgtcgggcgagtcaggatgttagcggctggttagcctggtatcagcagaaaccagggctagcccctcagctcctgatctttggtgcatccactttgcaaggtgaagtcccatcaagattcagcggcggtggatctgggacagattttactctcaccatcagcagcctgcagcctgaagattttgccacttattattgtcaacaggctaaatatttcccttacacttttggccaggggaccaagctggaaatcaaa

SEQ ID NO: 183 - protein, 244 aa, synthetic construct
QVQLQQSGPGLVKPSQTLSLTCAISGNSVSSNSAAWNWIRQSPSRGLEWLGRTYYRSKWYNDYAVSVKSRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGGGSGTDFTLTISSLQPEDFATYYCQQAKYFPYTFGQGTKLEIK

SEQ ID NO: 184 - protein, 18 aa, synthetic construct
GSTSGSGKPGSGEGSTKG

SEQ ID NO: 185 - protein, 5 aa, synthetic construct
GGGGS

SEQ ID NO: 186 - protein, 4 aa, synthetic construct
GFLG

SEQ ID NO: 187 - protein, 15 aa, synthetic construct
GGGGSGGGGSGGGGS

SEQ ID NO: 188 - protein, 4 aa, synthetic construct
RAKR

SEQ ID NO: 189 - protein, 25 aa, synthetic construct
GGGGSGGGGSGGGGSGGGGSGGGGS